(19) United States
(12) Patent Application Publication
(10) Pub. No.: US 2025/0191681 A1
(43) Pub. Date: Jun. 12, 2025

(54) METHODS OF CHARACTERISING A DNA SAMPLE

(71) Applicant: Cambridge Enterprise Limited, Cambridge (GB)

(72) Inventors: Andrea Degasperi, Cambridge (GB); Serena Nik-Zainal, Cambridge (GB)

(21) Appl. No.: 18/844,010

(22) PCT Filed: Mar. 9, 2023
(86) PCT No.: PCT/EP2023/056078

§ 371 Date: Sep. 4, 2024

(30) Foreign Application Priority Data
Mar. 10, 2022 (GB) 2203375.7

(51) Int. Cl. G16B 20/20; G16B 40/30

(52) U.S. Cl. CPC G16B 20/20; G16B 40/30

(57) ABSTRACT

The invention provides a method of characterising a DNA sample, the method including the steps of: obtaining a mutational catalogue for the sample, wherein a mutational catalogue comprises counts of mutations in a plurality of predetermined categories; obtaining a mutational signatures catalogue comprising a first set of one or more mutational signatures and a second set of one or more mutational signatures; determining a first set of exposures of the sample to the mutational signatures in the first set of mutational signatures; identifying at least one of the mutational signatures in the second set of mutational signatures that is likely to be present in the sample using the results of the determining; and providing an indication of which of the mutational signatures in the mutational signatures catalogue is present in the sample. Methods of providing a mutational signature catalogue, and related systems and products are also described.

### FIELD OF INVENTION

The present invention relates to a method for characterising a DNA sample. It is particularly, but not exclusively, concerned with a method for characterising a DNA sample in terms of the mutational signatures that are present in the sample, and methods for identifying mutational processes that are active in a cancer, and identifying treatments and prognosis accordingly.

### BACKGROUND TO THE INVENTION

The genome of a cancer is a highly distorted entity that has acquired thousands of genetic aberrations since conception. If examined comprehensively, cancer genomes can thus reveal insights regarding carcinogenesis (2).

Today, modern sequencing technologies have augmented the scale and rapidity of genome re-sequencing (3), permitting whole-genome sequencing (WGS) approaches that provide an all-inclusive perspective on cancer genomes (4). Beyond the handful of causative ‘driver’ mutations, WGS allows exploration of the full landscape of ‘passenger’ mutations that describe the processes that have arisen during tumorigenesis, resulting in patterns termed ‘mutational signatures’ (5-7). While drivers become important targets for therapeutic intervention, mutational signatures provide clues regarding historical environmental exposures and highlight potentially targetable pathway defects (4, 6, 8, 9).

Multiple catalogues of mutational signatures have been obtained to date using different data sets. For each such catalogue, it is possible to identify mutational signatures from the respective catalogue that are present in a sample. However, there is little understanding of the coverage and redundant information content between these different catalogues.

Therefore, there is still a need for improved methods for identifying mutational signatures in a sample.

### STATEMENTS OF INVENTION

The present inventors postulated that improved mutational signatures could be obtained by analysing large cohorts of WGS tumours. Whole-genome sequencing (WGS) permits comprehensive cancer genome analyses, revealing mutational signatures, imprints of DNA damage and repair processes that have arisen in each patient's cancer. They performed mutational signature analyses on 12,222 WGS tumor-normal matched pairs, and contrasted these results to two independent cancer WGS datasets, involving 18,640 WGS cancers in total. By analysing this data separately for each tumour type, they were able to identify 40 single and 18 double substitution signatures previously unidentified. Critically, they showed that for each organ, cancers have a limited number of ‘common’ signatures and a long tail of ‘rare’ signatures. They provided a practical solution for utilizing this concept of common versus rare signatures in future analyses.

Thus, according to a first aspect, there is provided a method of characterising a DNA sample, the method including the steps of: obtaining a mutational catalogue for the sample, wherein a mutational catalogue comprises counts of mutations in a plurality of predetermined categories; obtaining a mutational signatures catalogue comprising a first set of one or more mutational signatures and a second set of one or more mutational signatures; determining a first set of exposures of the sample to the mutational signatures in the first set of mutational signatures; identifying at least one of the mutational signatures in the second set of mutational signatures that is likely to be present in the sample using the results of the determining. The method may further comprise providing an indication of which of the mutational signatures in the mutational signatures catalogue is present in the sample. The method may be computer implemented.

The present inventors have identified that the ever-increasing number of mutational signatures poses the challenge of using mutational signature analysis in practice, whether in a new study of aggregated samples or for individual patients. To address this, they provide a signature ‘fitting’ process which utilizes a set of circumscribed signatures to ask which pre-defined signatures are present in their samples. This approach may be particularly useful in the context of signature sets which comprise a first set of signatures that are more likely to be present in a variety of samples than the signatures in the second set. This enables users to understand which mutational signatures are present in a new set of patient samples.

The method may have any one or more of the following features. The mutational signatures in the first set may be more likely to be present in the sample than the mutational signatures in the second set. The present inventors have identified that for any cohort of samples, there are mutational signatures that are more likely to be present in the samples (i.e. more frequent) and mutational signatures that are less likely to be present in the samples (i.e. rare/less frequent). They have further identified that fitting these signatures using a two step process where the more frequent signatures are fitted first, then additional signatures from the second set are fitted based on the results of the first fitting (such as e.g. if an additional signature improves the fit or adequately explains the unexplained portion of the mutational catalogue) performed significantly better (in terms of fit, i.e. explanation of the mutational catalogues) than attempting to fit all of these mutational signatures as a single catalogue (as is currently done in the art). This insight is applicable to any mutational signature catalogue and any sample, as the general insight that mutational signature catalogues contain signatures that are more or less frequent for a group of samples apply to any catalogue and any sample to which the catalogue is to be applied.

Identifying at least one of the mutational signatures in the second set of mutational signatures that is likely to be present in the sample using the results of the determining may comprise identifying one or more candidate mutational signatures in the second set that improve the fit of a mutational catalogue comprising the first set of mutational signature and the one or more candidate mutational signatures, compared to a mutational signature catalogue that does not comprise the candidate mutational signature.

Identifying at least one of the mutational signatures in the second set of mutational signatures that is likely to be present in the sample using the results of the determining may comprise: determining a second set of exposures of the sample to the mutational signatures in the first set of mutational signatures and a candidate mutational signature in the second set of mutational signatures; and determining that the candidate mutational signature in the second set of mutational signatures is likely to be present in the sample if the reconstruction error between the mutational catalogue and a reconstructed mutational catalogue associated with the second set of exposures satisfies one or more predetermined criteria. The one or more predetermined criteria may be selected from: the difference between the reconstruction error associated with the first set of exposures and the reconstruction error associated with the second set of exposures being below a predetermined value, and the candidate mutational signature being associated with the highest difference between the reconstruction error associated with the first set of exposures and the reconstruction error associated with the second set of exposures amongst all mutational signatures in the second set of mutational signatures. Thus, according to this embodiment, a candidate mutational signature in the second set is included in a set of signatures fitted to the sample if it sufficiently improves the fit of a mutational catalogue including the first set of mutational signatures and the candidate mutational signature, compared to a mutational catalogue not including the candidate mutational signature. An approach based on error reduction was identified by the inventors as having particularly good performance compared to an approach that fits all signatures simultaneously. The predetermined value may be a reduction in error of at least 5%, at least 10%, at least 15% or at least 20%.

Identifying at least one of the mutational signatures in the second set of mutational signatures that is likely to be present in the sample using the results of the determining may comprise: determining the residual between the mutational catalogue for the sample and a reconstructed catalogue corresponding to the determined exposures; and determining that a candidate mutational signature in the second set of mutational signatures is likely to be present in the sample if the similarity between the residual and the candidate mutational signature satisfies one or more predetermined criteria. The one or more predetermined criteria may be selected from: the similarity being above a predetermined threshold, and the similarity being highest amongst all mutational signatures in the second set of mutational signatures. Thus, according to this embodiment, a candidate mutational signature in the second set is included in a set of signatures fitted to the sample if the candidate mutational signature adequately explains the unexplained portion of the mutational catalogue after fitting a mutational signature catalogue comprising the first set of mutational signatures.

The determination of the exposure to one or more mutational signatures may be performed by identifying the matrix E that satisfies C˜PE where C is a mutational catalogue for one or more samples for which exposure is to be determined, P is a signature matrix comprising the one or more mutational signatures for which exposure is to be determined, and E is an exposure matrix. The determination of the exposure to one or more mutational signatures may be performed as described in Degasperi et al., 2020.

Determining the residual between the mutational catalogue for the sample and a reconstructed catalogue corresponding to the determined exposures may comprise using a constrained non-negative least squares approach to estimate the residual between the observed and reconstructed catalogs R=c-Se. The constraint(s) used may comprise that the reconstructed catalog Se should be mostly positive, −Se>−τ·Σi ci, with Σ=0.003 (or any other suitable value). The similarity may be a cosine similarity. The predetermined threshold on similarity may be 0.7, 0.8, 0.9, or any value between 0.7 and 0.99.

The method may further comprise determining a further set of exposures of the sample to the mutational signatures in the first set of mutational signatures and any mutational signatures in the second set of mutational signatures that is identified as likely to be present in the sample using the results of the determining. The method may further comprise excluding exposures that represent a proportion of the total sample mutations below a predetermined threshold. The predetermined threshold may be between 0 and 10%, about 1%, about 2%, about 3%, about 5% or about 10%. A mutational signature may be considered to be present in the sample if it is associated with an exposure above a predetermined threshold, or if it represents a number or proportion of mutations above a predetermined threshold. Using non zero thresholds for these criteria may advantageously reduce the risk of false positives.

The method may further comprise identifying one or more mutational processes present in the sample using at least one of the further set of exposures. Identifying one or more mutational processes present in the sample may comprise determining whether the exposures are indicative of the presence of a signature associated with the one or more mutational processes or a signature that maps to a signature associated with the one or more mutational processes. Examples of mutational processes associated with reference signatures are shown in Tables 12 and 13. Corresponding reference signatures are defined in Tables 14 and 15. Organ specific signatures are defined in Tables 18 and 19 and conversion matrices to convert these to the reference signatures of Tables 12-15 are provided in Tables 16 and 17. For example, signature DBS1 was shown to be associated with UV light exposure, signature DBS2 was shown to be associated with smoking, signatures DBS5, DBS18 were shown to be associated with prior platinum therapy, signature DBS11 was shown to be associated with APOBEC, signature SBS10d was shown to be associated with polymerase δ (POLD) dysfunction, signature SBS10a was shown to be associated with polymerase ε (POLE)-disfunction, SBS2 and SBS13 are due to APOBEC-related deamination, SBS96 was shown to be associated with mutations in MBD4 (where such tumors have sensitivities to checkpoint therapies), signature SBS105 was shown to be associated with deamination at CpGs followed by generic misincorporation during DNA replication and/or repair, signatures SBS18, SBS108, SBS30 were associated with compromised base excision repair, signatures SBS6, SBS15, SBS26, and SBS44 were shown to be associated with MMR deficiency, signature SBS14 was shown to be associated with MMRd and POLE dysfunction, signature SBS20 was shown to be associated with MMRd and POLD dysfunction, signature SBS3 was shown to distinguish BRCA1/BRCA2-null from sporadic breast cancers, signature SBS8 was shown to be increased in BRCA1/BRCA2-null cancers, signature SBS129 was shown to be associated with somatic TP53 mutations, SBS22 is due to aristolochic acid, SBS31 is associated with prior platinum exposure, DBS5 and DBS18 are associated with prior platinum exposure, SBS4 was shown to be associated with tobacco smoke exposure (and may indicate a metastatic lesion of lung primary when observed in non-lung cancer), SBS11 was shown to be associated with alkylation on a mismatch repair deficient background, SBS90 was shown to be associated with duocarmycin, SBS88 was shown to be associated with colibactin produced by pks+E. coli infection, SBS11 was shown to be associated with temozolomide on an MMR-deficient genetic background, SBS14 was shown to be associated with MMRd, UV-induced SBS7a was shown to be UV-induced (and may be indicative of metastatic lesions if e.g. in CNS cancer.

Identifying at least one of the mutational signatures in the second set of mutational signatures that is likely to be present in the sample using the results of the determining may comprise identifying a single candidate mutational signature in the second set that improves the fit of a mutational catalogue comprising the first set of mutational signature and the candidate mutational signature, compared to a mutational signature catalogue that does not comprise the candidate mutational signature. The present inventors have discovered that cancer samples usually have one or zero rare signatures present in addition to a few (median of five) common signatures. Thus, fitting a full set of rare signatures as part of a mutational signatures catalogue is likely to overfit the data resulting in poor identification of signatures present in the sample. By contrast, the approaches described herein enable the confident identification of the rare mutational signatures (if any) that are present in a sample.

The signatures in the first and/or second set of mutational signatures may be mutational signatures that have been extracted from organ-specific cohorts of samples. The present inventors have shown that performing signature extraction on an organ-specific (i.e. separately for each cohort of samples grouping samples originating from the same organ) may result in more reliable and stable mutational signatures. COSMIC and/or Reference Signatures (such as those described in Degasperi et al., 2020) are a simplified means of discussing signatures that are mutually present across tissues. However, they are purely mathematical constructs-an averaged result across different organs-thus organ-specific signatures are more likely to be accurate biological representations of the mutational processes that occur within a tissue. The first set of mutational signatures may be mutational signatures that are specific to the organ from which the sample originates. The present inventors have demonstrated that using organ-specific common signatures rather than corresponding reference signatures improved the accuracy of signature assignment. Signatures may be considered to be specific to an organ when they have been extracted from a cohort of samples primarily comprising samples originating from the organ. Such a cohort of samples may comprise at most 10%, at most 5%, preferably no samples that do not originate from the organ. Examples of organ-specific signatures are provided in Tables 18 and 19.

In embodiments, the first set of mutational signatures are selected from the common organ-specific mutational signatures listed in Table 20 (with reference to Table 18). In embodiments, the second set of mutational signatures are selected from the rare mutational signatures listed in Table 20.

The second set of mutational signatures may be mutational signatures that are not already represented in the first set of mutational signatures and that have been extracted in at least two independent extractions from respective cohorts of samples. The two independent extractions may be extractions performed on two different organ-specific cohorts. The inventors have found that rare signatures, high-quality reference signatures observed as rare signatures across the various organs and cohorts at least twice, and that did not already belong to the set common signatures were particularly useful. The signatures may have been extracted in at least two independent extractions performed on two different organ-specific cohorts, where the cohorts may comprise samples from the same or different organs.

The sample may be a tumor sample or a sample derived therefrom, optionally wherein the sample is from a tumour type selected from: skin, lung, stomach, colorectal, bladder, liver, uterus, ovary, biliary, kidney, pancreas, breast, prostate, bone/soft-tissue, central nervous system (CNS), lymphoid, oropharyngeal, neuroendocrine tumors (NET), and myeloid tumour. An organ-specific cohort may be a cohort comprising samples selected from a single one of: skin, lung, stomach, colorectal, bladder, liver, uterus, ovary, biliary, kidney, pancreas, breast, prostate, bone/soft-tissue, central nervous system (CNS), lymphoid, oropharyngeal, neuroendocrine tumors (NET), and myeloid tumour.

The mutational catalogue for the sample has been derived from sequence data for the sample. The method may comprise obtaining sequence data for the sample and deriving the mutational catalogue by counting mutations within each of the predetermined categories. The mutational catalogue and/or the mutational signatures catalogue may have been determined from whole genome sequencing data. The methods described herein are applicable to mutational catalogues that have been obtained from any sequencing approach that allows identification of mutations over a substantial part of the genome. This may be achieved through whole genome sequencing (WGS), whole exome sequencing or any capture sequencing approach (i.e. targeted/bait based sequencing) that captures a portion of the genomes such as e.g. at least 10% of the genome, at least 20% of the genome, at least 30% of the genome, at least 40% of the genome, at least 50% of the genome, at least 60% of the genome at least 10% of the exome, at least 10% of the exome, at least 20% of the exome, at least 30% of the exome, at least 40% of the exome, at least 50% of the exome, at least 60% of the exome, at least 100genes, at least 200 genes, at least 300 genes, at least 400 genes, at least 500 genes, and/or at least 1000 genes. Further, when incomplete genome sequencing is used, some of the genome may be imputed based e.g. on comparison with corresponding sequences in more complete profiles. In embodiments, the mutational catalogue and/or the mutational signatures catalogue has been determined from whole genome sequencing data or whole exome sequencing data. The present inventors have identified that the power to accurately discern mutational signatures is orders of magnitude greater using a pure WGS dataset when compared to other sequencing strategies. The genomic footprint for whole exomes (WES) is 100-fold lower and 2,000-4,000-fold lower in targeted sequencing (TS) experiments. Analyzing solely WGS cancers, rather than pooling data from diverse sequencing strategies, also avoids issues related to differing AT/GC representation in WES/TS data, which influence signature extractions.

The mutational catalogue may comprise the counts of the number of somatic mutations for each of a plurality of categories of single base substitutions or double base substitutions. The mutational catalogue may be a 96 channel SBS profile or a 78 channel DBS profile. The mutational catalogue may comprise the counts of the number of somatic mutations for each of a plurality of categories of insertions and/or deletions. The mutational catalogue may comprise the counts of the number of somatic mutations for each of a plurality of categories of rearrangements. The methods described herein are equally applicable to base substitutions, insertions, deletions and rearrangements.

The mutational signatures in the first set and/or in the second set may be mutational signatures that have been validated by cross reference with at least one independently extracted mutational signature catalogue. In the examples provided herein, the inventors demonstrate the use of an agnostic three-way signature comparison in 16 tissue types that were present in all three cohorts. They show that signatures from the same organ in different cohorts were more similar to each other than to those in other tissue type, providing evidence that mutational signatures in each organ are highly reproducible, have tissue-specificities, and were detectable regardless of sequencing platform or mutation-calling algorithms. Second, the use of multiple independent cohorts helps to validate signatures found in single organs, and that could otherwise be mistaken for other signatures or considered artefactual. Validating signatures may comprise mapping signatures extracted from one cohort to signatures obtained from another cohort using a metric of similarity such as cosine similarity.

The mutational signatures in the first set of signatures may have been identified by extracting mutational signatures from a cohort of samples which has been separated into a first group and a second group of samples, the first group comprising samples with mutational profiles that are more common than the mutational profiles of the samples in the second group, wherein the first set of signatures have been identified by extracting mutational signatures from the cohort of samples excluding the second group of samples. The mutational signatures in the second set of signatures may have been identified in one or more cohorts of samples that are different from the cohort of samples from which the first set of signatures have been identified, or by extracting mutational signatures from one or more samples in the second group of samples using the first set of mutational signatures as constant in the extraction process. In the present work, the inventors introduce the notion of common and rare signatures and show that focusing on common mutational profiles to extract common signatures has produced signatures that are highly reproducible across cohorts. Note that the terms common/rare are relative to a particular cancer or group of cancers (i.e. a particular cohort of samples). The terms common and rare refer to the step at which the signature was identified in a specific organ. In practice, a specific mutational pattern could be considered rare in one per organ extraction of one cohort and be a common pattern in another. In other words, the present inventors have identified that by excluding samples with unusual profiles in a first extraction step, the number of mutational signatures in the initial set was limited to common patterns, reducing the mixing of signatures in the extraction process. The first set of signatures may have been identified by extracting mutational signatures from a cohort of samples which has been separated into a first group and a second group of samples by clustering the mutational catalogues for the samples. This may be performed using hierarchical clustering. Hierarchical clustering may be performed using average linkage and/or 1-cosine distance as a similarity measure. In the present disclosure, the inventors propose a new approach to signature extraction where they cluster a mutational catalogue, select samples with recurrent profiles, and perform signature extraction on these. In other words, cases with unusual profiles and likely to have rare signatures are excluded in the first extraction. They show that this yielded a set of highly accurate ‘common signatures’ that are prevalent for that tumor type/cohort. Removing atypical samples in the first extraction step is believed to be especially useful for large cohorts, where very rare signatures may be present and could interfere with the accurate identification of common signatures. The cohort of samples may comprise at least 1000 samples, at least 2000 samples, at least 3000 samples, at least 5000 samples, or at least 10000 samples. These numbers may be applicable in the case of non-organ-specific cohorts. The cohort of samples may comprise at least 20 samples, at least 50 samples, or at least 100 samples. These numbers may be applicable in the case of organ-specific cohorts.

The first set of signatures may have further been extracted by identifying a first set of one or more clusters of mutational profiles that comprise mutational profiles that are more frequent than mutational profiles in a second set of one or more clusters, and extracting a set of signatures from mutational profiles in the mutational profiles in the first set of one or more clusters.

The extraction of the first set of mutational signatures may use non-negative matrix factorization (NMF). NMF may be used with Kullback-Leibler divergence (KLD) optimization, repeated bootstrapping (such as e.g. at least 300 bootstraps), and removal of local minima. For example, given a matrix of catalogs C, nonnegative matrix factorization (NMF) may be applied to 20 matrices C′, bootstrapped from C. The NMF may be solved using an algorithm (e.g. the Lee and Seung multiplicative algorithm (46)) that optimizes the Kullback-Leibler divergence (KLD). Solving the NMF may produce a matrix of signatures S and a matrix of exposures E for each NMF run, such that C′˜SE. The NMF may be repeated a number of times (such as e.g. at least 300 times) for each bootstrap matrix, using random initializations. A set of solutions may be selected solutions that have a final KLD within a predetermined percentage (e.g. 0.1%) of the best solution found (the solution with the lowest KLD).

Point estimates of exposures may be obtained as the median of the exposures obtained from bootstrapping. In embodiments, such as when the number of mutations were too low to perform the bootstrap-based fit described above (e.g. for DBS), a single signature fit instead. Exposures below a predetermined threshold, such as e.g. 5% of the total SBS burden or e.g. 25% of DBS burden per sample may be set to zero. This may advantageously reduce the risk of over-fitting.

One or more signatures in the second set of signatures may have been identified using a process comprising: separating a cohort of samples into a first group and a second group of samples, the first group comprising samples with mutational catalogue that are more common than the mutational profiles of the samples in the second group, identifying a first set of mutational signatures by extracting mutational signatures from the cohort of samples excluding the second group of samples, identifying one or more samples in the second group of samples with mutational profiles based on the reconstruction error associated with a mutation catalogue reconstructed using the first set of mutational signatures, and extracting one or more signatures from the identified one or more samples in the second group of samples. The reconstruction error for a sample may be obtained as the sum of absolute deviations between the mutational catalogue c of the sample and the reconstructed mutational catalogue Se obtained by fitting a mutational signature catalogue to the mutational profile of the sample, divided by the total mutations in the mutational catalogue of the sample

Identifying one or more samples in the second group of samples with mutational profiles based on the reconstruction error associated with a mutation catalogue reconstructed using the first set of mutational signatures may comprise: for each sample in the second group of samples, obtaining a sample residual error by estimating the sample exposures obtained by fitting the first set of mutational signatures to the mutational profile for the sample and using a least square estimation with a constraint that the difference between the observed and reconstructed catalogues should be above a predetermined threshold applied to the sum of coefficients in the reconstructed catalogue; and clustering the sample residual errors for the samples in the second group of samples, wherein the samples in a particular cluster are identified and used for signature extraction.

One or more signatures in the second set of signatures may have been identified using a process further comprising for each cluster of samples, extracting one signature using an extraction process constrained to use the signatures in the first set of signatures as constant, optionally wherein the extraction process using NMF, where the signature matrix S contains the first set of signatures as constants, and one additional column that is estimated to contain the new signature. For example, a sample residual error may be calculated using the constraint that the reconstructed profile should be mostly positive, which can be expressed as c-Se>−τ·Σi ci), where for example τ=0.003 or any other suitably low value. Clustering the sample residual errors may comprise using hierarchical clustering (e.g. with average linkage), with any suitable distance metric such as e.g. 1-cosine similarity as distance. The method may further comprise excluding from the second set of samples, any sample that has a residual error below a minimum number of mutations. For example, a minimum number of mutations between 3 and 400 mutations may be used for SBS. As another example, a minimum number of mutations between 40-50 mutations may be used for DBS. The minimum number of mutations may be chosen separately for each cluster. The predetermined number of mutations may be determined by obtaining the distribution of residuals for a cohort of samples and identifying a value that separates the samples with residuals above a level of background noise.

The one or more signatures in the second set of signatures have been identified by: performing a process as described above independently on at least two different cohorts of samples; and selecting signatures that are identified in at least two of the different cohorts. Extracting a first set of signatures may comprise extracting between 5 and 10 SBS in a cohort of samples. Extracting a second set of signatures may comprise extracting between 0 and 21 SBS in said cohort of samples. Extracting a first set of signatures may comprises extracting between 1 and 5 DBS in a cohort of samples. Extracting a second set of signatures may comprises extracting between 0 and 15 DBS in said cohort of samples. The cohort of samples may be an organ-specific cohort of samples.

Providing an indication of which of the mutational signatures in the mutational signatures catalogue is present in the sample may comprises: determining exposure to signatures in a mutational signature catalogue comprising the first set of signatures and at least one signature in the second set of signatures, and mapping the exposures to a first set and/or a second set of mutational signatures to a reference set of mutational signatures. The method may further comprise identifying one or more mutational processes likely to be active in the sample based on the mapping. Providing an indication of which of the mutational signatures in the mutational signatures catalogue is present in the sample may comprises: determining exposure to signatures in a mutational signature catalogue comprising the first set of signatures and at least one signature in the second set of signatures, and using one or more exposures as an input to a method for determining whether the DNA sample is from a tumour that has a deficiency in a DNA repair pathway. Providing an indication of which of the mutational signatures in the mutational signatures catalogue is present in the sample may comprises:

determining exposure to signatures in a mutational signature catalogue comprising the first set of signatures and at least one signature in the second set of signatures, and using one or more exposures as an input to a method for determining whether the DNA sample is from a tumour that has a characteristic that is indicative or prognosis or response to therapy. Providing an indication of which of the mutational signatures in the mutational signatures catalogue is present in the sample may comprises: determining exposure to signatures in a mutational signature catalogue comprising the first set of signatures and at least one signature in the second set of signatures, and providing the one or more exposures or metrics derived therefrom as part of a report characterising a tumour from which the DNA sample has been obtained.

Mapping to reference signatures may comprise used a conversion matrix to convert the signature exposures (such as e.g. cohort-organ signature exposures) into reference signature exposures. Examples reference signatures and conversion matrices are provided in Tables 16 and 17. The reference signatures may have been obtained by identifying a mutational signature catalogue independently in at least two different cohorts of samples using the methods described above, and clustering the independently obtained mutational catalogues to identify clusters of mutational signatures that are more similar to each other than to signatures in other clusters. For examples, mutational signatures that have a similarity above a predetermined threshold (such as e.g. a cosine similarity above 0.8) may be considered to form a cluster of similar mutations. Such clusters may be further separated into distinct clusters, or multiple clusters may be combined into a single clusters, based on the level of noise in signatures within the clusters. For example, the level of noise may be quantified using the spread of the signature signal across channels. A signature that only contains a few distinct peaks may be considered to have low noise, whereas a signature that contains many peaks (potentially of similar size to a signature of the first type) may be considered to have high noise. On or more reference signatures may be identified as a summarised signature (e.g. cluster average) of a respective mutational signature cluster. These may be referred to as “distinct patterns”. The method may further comprise assigning each summarised signature to one of 3 groups: i) a true signature, thus observable in independent extractions of diverse organs and cohorts (recurrent pattern); ii) a mix of other signatures (mixed pattern); iii) a pattern seen in only one extraction (singleton pattern). Recurrent distinct patterns may be additionally clustered to remove patterns that may simply be a variant of another pattern. Mixed distinct patterns that can be estimated as a combination of two distinct patterns using non-negative least squares may be excluded. Singleton distinct patterns may be dismissed if they were variants of other reference signatures. This may be assessed by obtaining the similarity (e.g. cosine similarity) between a singleton distinct pattern and other distinct patterns, and comparing the singleton distinct pattern with the most similar distinct patterns (such as e.g. the 1, 2, 3 or 5 most similar distinct patterns) to determine whether the singleton distinct pattern includes one of these distinct patterns and noise. In such cases the singleton may be mapped to the identified distinct pattern.

When the mutational signatures are DBS signatures, a first and/or second set of signatures may have been extracted using a process comprising an additional step of excluding any DBS signature that comprise adjacent substitutions that are not in cis. This may exclude DBS that are simply the mathematical outcome of an associated SBS hypermutator. When the mutational signatures are DBS signature, first and/or second set of signatures may have been extracted using a process comprising an additional step of excluding any DBS signature that was correlated with an SBS signature extracted from the same cohort, and where the DBS pattern can be expected given the SBS pattern. These assessments were helpful in refuting several DBS signatures as being simply due to chance.

Thus, also described herein are methods of identifying one or more mutational processes likely to be present in a DNA sample, methods of determining whether a DNA sample is from a tumour that has a deficiency in a DNA repair pathway, methods for determining whether the DNA sample is from a tumour that has a characteristic that is indicative or prognosis or response to therapy and methods of characterising a tumour from which a DNA sample has been obtained, the methods comprising characterising the DNA sample using the methods described herein. Each of these methods may comprise providing the results of the characterising/identifying/determining to a user, for example as part of a report.

According to a further aspect, there is provided a method of providing a mutational signature catalogue, the method comprising: separating a cohort of samples into a first group and a second group of samples, the first group comprising samples with mutational profiles that are more common than the mutational profiles of the samples in the second group, and extracting a first set of mutational signatures from the cohort of samples excluding the second group of samples. The method according to the present aspect may comprise any of the steps described above. The method according to the present aspect may have any of the features described in relation to the preceding aspect.

According to a further aspect, there is provided a system comprising: a processor; and a non-transitory computer readable medium comprising instructions that, when executed by the processor, cause the processor to perform the (computer-implemented) steps of the method of any preceding aspect. According to a further aspect, there is provided a non-transitory computer readable medium or media comprising instructions that, when executed by at least one processor, cause the at least one processor to perform the method of any embodiment of any aspect described herein. According to a further aspect, there is provided a computer program comprising code which, when the code is executed on a computer, causes the computer to perform the method of any embodiment of any aspect described herein.

### BRIEF DESCRIPTION OF THE FIGURES

FIG. 1 is a flow diagram showing, in schematic form, a method of characterising a DNA sample according to the disclosure.

FIG. 2 shows an embodiment of a system for characterising a DNA sample.

FIG. 3 is a schematic representation of the workflow of mutational signature analysis demonstrated in the disclosure. Three cohorts (GEL, ICGC and Hartwig) were evaluated independently. For each organ in each cohort, mutational catalogues were clustered, where samples with atypical catalogues were excluded from the extraction process. Samples with similar catalogues were subjected to signature extraction to obtain a set of common organ-specific signatures. These common signatures were fitted into all samples, highlighting samples that had a high error profile that were subsequently used to identify rare signatures. Pie chart shows the total number of SBS signatures identified for each independent extraction of each organ in all three cohorts.

FIG. 4 shows the number of common and rare SBS signatures in each cohort (top), and the number of common (middle) and rare SBS signatures as a function of number of samples analyzed (bottom).

FIG. 5 shows a procedure to determine Reference Signatures from all the cohort-organ signatures identified. Numbers refer to the SBS signatures analysis.

FIG. 6 shows the SBS signatures identified across 18,640 WGS cancers. (A) Frequency of SBS signatures in the present disclosure. Orange bars highlight 42 signatures reported in this study and present in COSMIC v3.2. Blue bars highlight 40 previously unreported signatures found in the present analysis. (B) Same information as A with log scale on y-axis.

FIG. 7 shows the frequency of DBS signatures in the present disclosure.

FIG. 8 shows the correlation of DBS with SBS exposures across cohorts. Numbers in each column report the number of organs implicated in the correlative analyses. A correlation is computed independently for each organ and the correlations are displayed as a boxplot. Boxplots denote median (horizontal line) and 25th to 75th percentiles (boxes). The lower and upper whiskers extend 1.5×IQR (IQR: inter-quartile range).

FIG. 9 illustrates the concept of common and rare mutational signatures in cancer samples and the principle of identifying mutational signatures in a sample according to a method described herein (fitMS). Schematic depiction of common (gray and lighter colors) versus rare signatures in three example tumor-types (breast, central nervous system (CNS) and colorectal cancers). Each patient could have different amounts of some (or all) of the common signatures. Occasionally, a patient may carry a rare signature as well (bright colors). Some common signatures are ubiquitous and present in nearly all tumor-types while some common signatures may be restricted to some tumor-types. Rare signatures may be unique (for example, yellow dot) or could also occur in other tumor-types (for example, red dots). We propose a practical package, FitMS that utilizes the insights obtained through this work. Given a new sample, for example, a new brain cancer WGS mutation catalog, FitMS will fit common CNS signatures before attempting to discover additional rare signatures seen in CNS and other tumours.

FIG. 10 illustrates the extraction of common and rare signatures in the ICGC Breast cancer cohort, as an example of the methodological steps. (A) Mutational catalogues from the ICGC-Breast cohort were clustered using hierarchical clustering with average linkage and 1-cosine similarity as distance. (B) The averaged profiles of the catalogues in each of the nine clusters identified in (A), with n indicating the number of catalogues in each cluster. The red box highlights the clusters that were used to extract common signatures. These include the three largest clusters and two smaller clusters that appear to contain the well-reported SBS17. Samples in clusters 6-9 were excluded from the extraction of common signatures. (C) Common signatures obtained using our extraction framework from the samples highlighted in (B). (D) Distribution of the error between the original catalogues and the reconstructed catalogues modelled using the extracted common signatures. The error is given by the sum of absolute deviations between the original and reconstructed catalogues, divided by the number of mutations in the samples. The error was modelled using a normal distribution and red dots show significantly greater error values (p-value less than 0.03). (E) Residual mutations of example sample PD5937a, obtained as the difference between the original catalogue and a reconstructed catalogue modelled using the common signatures. The exposures of the reconstructed catalogue were obtained using non-negative least squares with the additional constraint that the residual should be mostly positive (Materials and Methods). (F) Residuals are clustered using hierarchical clustering with average linkage and 1-cosine similarity as distance. (G) Average pattern of residual clusters in (F). (H) For each cluster of residuals in (G), a rare signature was extracted using a variant of NMF where a signature can be estimated while holding the common signatures as constant.

FIG. 11 shows evidence of organ-specificity of signatures across three cohorts. (A) Example of how to seek ‘proportion of matching signatures’. For each common organ-specific signature in an organ (organ 1) in a first cohort (cohort A), the single most similar common organ-specific signature in the second cohort (cohort B) is sought. The proportion of matching signatures is the proportion of signatures in the organ in the first cohort that have the most similar signatures in a given organ in the second cohort. For example, in panel (A), the proportion of matching signatures from cohort A organ 1 to cohort B organ 2 is ⅗=0.6, while from cohort A organ 1 to cohort B organ 3 is ⅕=0.2. Some signatures may be unmatched if there is no signature in cohort B with a cosine similarity of at least 0.85. (B) Example of a comparison of signatures between the same tissue-type (breast vs breast) where there are six possible comparison outcomes (GEL-ICGC, GEL-Hartwig, ICGC-Hartwig, ICGC-GEL, Hartwig-GEL, Hartwig-ICGC), and thus six values of proportion of matching signatures. (C) Example of a comparison between different organs (breast vs ovary), where there are 12 possible comparisons. (D) After considering all possible cohort and organ combinations, a Tukey test is used to determine if the same organ comparison exhibits the greatest similarity than each of the other 15 inter-organ comparisons (confidence level 0.95, p-value threshold 0.05). (E) Agnostic three-way comparison of the common organ-specific signatures across all three cohorts in 16 organs. We use *** to indicate that the proportion of matching signatures for the same organ comparison (for example Breast to Breast) is significantly higher than all 15 other organ comparisons, and * higher than 7, according to the Tukey test in (D). (F) Prostate-specific SBS5 signatures from the 3 three cohorts are shown next to Reference Signature (RefSig) SBS5 and COSMIC SBS5. RefSig SBS5 is obtained as an average of cohort-organ signatures, see text. (G) Cosine similarity of signatures shown in (F). Colors are used to highlight groups of signatures, with orange used if the cosine similarity is greater than or equal to 0.95.

FIG. 12 shows the results of identification of organ-specific and reference signatures and comparison to literature. (A) Number of common and rare DBS signatures in each cohort. (B) Common DBS signatures as a function of number of samples analyzed. (C) Rare DBS signatures as a function of number of samples analyzed. (D) Venn diagram comparing SBS COSMIC signatures version 3.2 and the SBS reference signatures identified in this study. (E) Cosine similarity of the 42 SBS signatures in the Venn diagram intersection in (D). (F) Venn diagram comparing DBS COSMIC signatures version 3.2 and the DBS reference signatures identified in this study. (G) Cosine similarity of the 9 DBSs in the Venn diagram intersection in (F).

FIG. 13 illustrates the process of fitting mutational signatures according to a method described herein: how to use common and rare signatures for signature assignments. Mutational signature fit with FitMS. (A) Numbers of common and rare SBS signatures found in each sample across the three cohorts. (B) Pie charts indicating the proportion of samples with one or more SBS rare signatures. (C) FitMS can be applied to individual new samples as a two-step approach. In the first step, common organ-specific signatures that were obtained from the same tissue of origin as the new sample are used. In a second step, FitMS attempts to identify additional rare signatures that may be present. (D) Simulation study sensitivity of two FitMS implementations (constrainedFit and errorReduction) and a simple “fit all” approach. (E) Same simulations as (D), F1 score. (F) Same simulations as (D), root mean squared error (RMSE). (G) Simulation study sensitivity of the best FitMS strategy (errorReduction) and “fit all”, using either the organ-specific signatures (organSigs) or the reference signatures (RefSigs) as common signatures. (H) Same simulations as (G), F1 score. (I) Same simulations as (G), RMSE. Error bars are standard error of the mean, number of repeats is n=5.

FIG. 14 illustrates schematically the general workflow applied in the work described in the examples.

FIG. 15 shows the SBS reference signatures that have been previously reported (COSMIC v3.2). Previously reported SBS reference signatures identified in this study are depicted, where the corresponding pie-charts report the prevalence of the respective signatures in the samples across all tumour types, in the three cohorts (GEL, ICGC and Hartwig). “Samples” indicates total number of samples across the three cohorts with the signature. “Extractions” indicates the number of independent extractions that the signature was found in (note that extractions are performed for every organ independently in the three cohorts).

FIG. 16 shows the previously unreported SBS reference signatures identified in this study. Previously unreported SBS reference signatures identified in this study and their distribution in the samples across cohorts and organs. “Samples” indicates total number of samples across the three cohorts with the signature. “Extractions” indicates the number of independent extractions that the signature was found in (note that extractions are performed for every organ of three cohorts).

FIG. 17 shows the DBS reference signatures. Previously known (COSMIC v3.2) (A) and previously unreported (B) DBS signatures identified in this study and their distribution across cohorts and organs. “Samples” indicates total number of samples across the three cohorts with the signature. “Extractions” indicates the number of independent extractions that the signature was found in (note that extractions are performed for every organ of three cohorts). Where alignment data were available, next to the name we indicate whether the double substitutions were observed to be in cis or otherwise, the latter would suggest that the DBS signature is a false positive signature.

### DETAILED DESCRIPTION

In describing the present invention, the following terms will be employed, and are intended to be defined as indicated below.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

A “sample” as used herein may be a cell or tissue sample (e.g. a biopsy), a biological fluid, an extract (e.g. a protein or DNA extract obtained from the subject), from which genomic material can be obtained for genomic analysis, such as genomic sequencing (whole genome sequencing, whole exome sequencing, targeted (also referred to as “panel”) sequencing). In particular, the sample may be a blood sample, or a tumour sample. The sample may be one which has been freshly obtained from a subject or may be one which has been processed and/or stored prior to making a determination (e.g. frozen, fixed or subjected to one or more purification, enrichment or extractions steps). In particular, the sample may be a cell or tissue culture sample. As such, a sample as described herein may refer to any type of sample comprising cells or genomic material derived therefrom, whether from a biological sample obtained from a subject, or from a sample obtained from e.g. a cell line. The sample is preferably from a mammalian (such as e.g. a mammalian cell sample or a sample from a mammalian subject, including in particular a model animal such as mouse, rat, etc.), preferably from a human (such as e.g. a human cell sample or a sample from a human subject). Further, the sample may be transported ad/or stored, and collection may take place at a location remote from the genomic sequence data acquisition (e.g. sequencing) location, and/or the computer-implemented method steps may take place at a location remote from the sample collection location and/or remote from the genomic data acquisition (e.g. sequencing) location (e.g. the computer-implemented method steps may be performed by means of a networked computer, such as by means of a “cloud” provider).

A “tumour sample” refers to a sample that contains tumour cells or genetic material derived therefrom. The tumour sample may be a cell or tissue sample (e.g. a biopsy) obtained directly from a tumour. A tumour sample may be a sample that comprises tumour cell or genetic material derived therefrom, that has not be obtained directly from a tumour. For example, a tumour sample may be a sample comprising circulating tumour cells or circulating tumour DNA. Thus, a tumour sample may also be a biological fluid (e.g. a liquid biopsy such as a blood, urine, or cerebrospinal fluid biopsy). A sample comprising a mixture of tumour cells and other cells (or material genetic derived therefrom) may be subject to one or more processing steps, whether prior to or subsequent to the acquisition of sequence data, in order to identify sequence data that is representative of the genetic material from the tumour. For example, a sample comprising cells may be subject to one or more cell purification steps which selectively enrich the sample for tumour cells. Similarly, a sample comprising modified and non-modified cells can be subject to one or more purification or selection steps to enrich the sample for modified cells. Protocols for doing this are known in the art. As another example, a sample of genetic material may be subject to one or more capture and/or size selection steps to selectively enrich the sample for tumour-derived genetic material. Protocols for doing this are known in the art. As another example, sequence data may be subject to one or more filtering steps (e.g. based on fragment length) to enrich the data for information that relates to tumour-derived genetic material. Protocols for doing this are known in the art.

A “normal sample” (also referred to as “germline sample” or “parent sample”) refers to a sample that contains non-tumour or non-modified cells or genetic material derived therefrom. A normal sample may be matched to a particular tumour or modified sample in the sense that it is obtained from the same biological source (subject or cell line) as the tumour or modified sample. A normal sample may be a cell or tissue sample obtained from a subject, or a sample of biological fluid. A sample comprising a mixture of normal cells and other cells (or material genetic derived therefrom) may be subject to one or more processing steps, whether prior to or subsequent to the acquisition of sequence data, in order to identify sequence data that is representative of the genetic material from the normal cells (as already described above). For example, a sample comprising modified and non-modified cells can be subject to one or more purification or selection steps to enrich the sample for non-modified cells. Similarly, a sample comprising normal and tumour-derived cells can be subject to one or more purification steps which selectively enrich the sample for normal cells.

The term “sequence data” refers to information that is indicative of the presence and/or amount of genomic material in a sample that has a particular sequence. Such information may be obtained using sequencing technologies, such as e.g. next generation sequencing (NGS, such as e.g. whole exome sequencing (WES), whole genome sequencing (WGS), or sequencing of captured genomic loci (targeted or panel sequencing)), or using array technologies, such as e.g. SNP arrays, or other molecular counting assays. When NGS technologies are used, the sequence data may comprise a count of the number of sequencing reads that have a particular sequence. When non-digital technologies are used such as array technology, the sequence data may comprise a signal (e.g. an intensity value) that is indicative of the number of sequences in the sample that have a particular sequence, for example by comparison to an appropriate control. Sequence data may be mapped to a reference sequence, for example a reference genome, using methods known in the art (such as e.g. Bowtie (Langmead et al., 2009)). Thus, counts of sequencing reads or equivalent non-digital signals may be associated with a particular genomic location. Further, a genomic location may contain a mutation, in which case counts of sequencing reads or equivalent non-digital signals may be associated with each of the possible variants (also referred to as “alleles”) at the particular genomic location. The process of identifying the presence of a mutation at a particular location in a sample is referred to as “variant calling”, and can be performed using methods known in the art (such as e.g. the GATK HaplotypeCaller, https://gatk.broadinstitute.org/hc/en-us/articles/360037225632-HaplotypeCaller). For example, sequence data may comprise a count of the number of reads (or an equivalent non-digital signal) which match a germline (also sometimes referred to as “reference”) allele at a particular genomic location, and a count of the number of reads (or an equivalent non-digital signal) which match a mutated (also sometimes referred to as “alternate”) allele at the genomic location.

The term “mutation” refers to a difference in a nucleotide sequence (e.g. DNA or RNA) in a sample compared to a reference. For example, a mutation may be a single nucleotide variant (SNV), multiple nucleotide variants, a deletion mutation, an insertion mutation, a translocation, a missense mutation, a translocation, a fusion, etc. Mutations may be identified using sequence data. An “indel mutation” (or simply “indel”) refers to an insertion and/or deletion of bases in a nucleotide sequence (e.g. DNA or RNA) of an organism.

Within the context of the present invention, a mutation is typically a somatic mutation, unless the context indicates otherwise. A “somatic mutation” is a mutation that is present in a tumour or modified cell (or genetic material derived therefrom), but not in a corresponding (matched) normal or non-modified cell.

The present disclosure relates in part to method of identifying mutational signatures in a mutational catalogue that are present in a sample. A mutational signature catalogue is a set of mutational signatures. A mutational signature is a characteristic combination of mutation types that arises from one or more underlying mutational processes. Mutational processes may be endogenous (such as e.g. DNA repair pathway deficiencies) or exogenous (such as e.g. exposure to genotoxins). Mutational signatures can be extracted from cohorts of samples, by identifying characteristic combination of mutation types that best explain the mutational profiles of the samples in the cohort. This process also results in the quantification of “exposures” to each of the signatures, which quantify the extent of the effect of the respective signatures on the respective mutational profiles.

A mutational signature catalogue can be extracted from a plurality of mutational catalogues each associated with respective samples. A mutational catalogue can comprise mutational signatures extracted separately from a plurality of respective cohorts of mutational catalogues. A mutational catalogue comprises the number of mutations present in a sample within each of a plurality of mutation categories. A mutational catalogue can be seen as a summary of a list of mutations present in a sample, categorised according to a predetermined set of mutation categories. A list of somatic mutations or a mutational catalogue derived therefrom may comprise mutations of one or more types selected from: substitutions, rearrangements, deletions, and insertions (sometimes collectively referred to as “indels”). A mutational catalogue summarising somatic substitutions associated with a sample or a group of samples may be referred to as a “substitution profile”. Substitutions may be single nucleotide substitutions (also referred to as single base substitutions, SBS), double nucleotide substitutions (also referred to as double base substitutions, DBS), or triple nucleotide substitutions (also referred to as triple base substitutions, TBS). The plurality of categories in the context of substitutions may refer to the identity of the germline and mutated bases, and/or to the context of the mutated bases (identity of the one or more nucleotides flanking the mutated bases). In particular, in the context of SBS, the plurality of categories may refer to the identity of the germline and mutated base, and the identity of the 5′ and 3′ flanking bases. Thus, such categories may include one or more of the following categories, or categories that combine some of the following categories such as based on a common context and/or substitution: A[C>A]A, A[C>A]C, A[C>A]G, A[C>A]T, C[C>A]A, C[C>A]C, C[C>A]G, C[C>A]T, G[C>A]A, G[C>A]C, G[C>A]G, G[C>A]T, T[C>A]A, T[C>A]C, T[C>A]G, T[C>A]T, A[C>G]A, A[C>G]C, A[C>G]G, A[C>G]T, C[C>G]A, C[C>G]C, C[C>G]G, C[C>G]T, G[C>G]A, G[C>G]C, G[C>G]G, G[C>G]T, T[C>G]A, T[C>G]C, T[C>G]G, T[C>G]T, A[C>T]A, A[C>T]C, A[C>T]G, A[C>T]T, C[C>T]A, C[C>T]C, C[C>T]G, C[C>T]T, G[C>T]A, G[C>T]C, G[C>T]G, G[C>T]T, T[C>T]A, T[C>T]C, T[C>T]G, T[C>T]T, A[T>A]A, A[T>A]C, A[T>A]G, A[T>A]T, C[T>A]A, C[T>A]C, C[T>A]G, C[T>A]T, G[T>A]A, G[T>A]C, G[T>A]G, G[T>A]T, T[T>A]A, T[T>A]C, T[T>A]G, T[T>A]T, A[T>C]A, A[T>C]C, A[T>C]G, A[T>C]T, C[T>C]A, C[T>C]C, C[T>C]G, C[T>C]T, G[T>C]A, G[T>C]C, G[T>C]G, G[T>C]T, T[T>C]A, T[T>C]C, T[T>C]G, T[T>C]T, A[T>G]A, A[T>G]C, A[T>G]G, A[T>G]T, C[T>G]A, C[T>G]C, C[T>G]G, C[T>G]T, G[T>G]A, G[T>G]C, G[T>G]G, G[T>G]T, T[T>G]A, T[T>G]C, T[T>G]G, and T[T>G]T. In particular, in the context of DBS, the plurality of categories may refer to the identity of the germline and mutated bases. Thus, such categories may include one or more of the following categories, or categories that combine some of the following categories such as based on a common first and/or second position substitution: AA>CC, AA>CG, AA>CT, AA>GC, AA>GG, AA>GT, AA>TC, AA>TG, AA>TT, AC>CA, AC>CG, AC>CT, AC>GA, AC>GG, AC>GT, AC>TA, AC>TG, AC>TT, AG>CA, AG>CC, AG>CT, AG>GA, AG>GC, AG>GT, AG>TA, AG>TC, AG>TT, AT>CA, AT>CC, AT>CG, AT>GA, AT>GC, AT>TA, CA>AC, CA>AG, CA>AT, CA>GC, CA>GG, CA>GT, CA>TC, CA>TG, CA>TT, CC>AA, CC>AG, CC>AT, CC>GA, CC>GG, CC>GT, CC>TA, CC>TG, CC>TT, CG>AA, CG>AC, CG>AT, CG>GA, CG>GC,

CG>TA, GA>AC, GA>AG, GA>AT, GA>CC, GA>CG, GA>CT, GA>TC, GA>TG, GA>TT, GC>AA, GC>AG, GC>AT, GC>CA, GC>CG, GC>TA, TA>AC, TA>AG, TA>AT, TA>CC, TA>CG, and TA>GC. Similarly, in the context of TBS, the plurality of categories may refer to the identity of the germline and mutated bases. Thus, such categories may include one or more of each of the categories corresponding to all possible triple base substitutions such as TTT>AAA, TTT>GAA, etc., or categories that combine some of these categories such as based on a common first and/or second and/or third position substitution. A mutational catalogue summarising somatic deletions associated with a sample or a group of samples may be referred to as a “deletion profile”. A mutational catalogue summarising somatic insertions associated with a sample or a group of samples may be referred to as a “insertion profile”. A mutational catalogue summarising a list of mutations comprising both somatic insertions and deletions associated with a sample or group of samples may be referred to as an “indel profile”. An insertion or deletion may be referred to as “repeat mediated” if it occurs in a repetitive region. A repetitive region may be defined as a region that includes a plurality (e.g. 2 or more) of repeats of a sequence motif. A sequence motif may be defined as a sequence of between 1 and n bases, where n may be selected as 3, 4, 5, 6, 7, 8, 9, 10, 11, or 12. For example n=9 may be convenient. The use of higher values of n requires more extensive cataloguing of such regions, which may be associated with diminishing returns as repeats of longer motifs are less likely. A repetitive region may be defined by reference to a reference genome. In other words, a repetitive region may be defined as a particular locus (defined by its genomic coordinates) in a reference genome. Thus, any mutation identified within such a locus may be considered to be “repeat mediated”. Methods for obtaining an indel catalogue and deriving indel signatures from such a catalogue are described in Nik-Zainal et al. (Nature. 2016 May 2; 534(7605): 47-54) and in Degasperi et al. (Nat Cancer. 2020 February; 1(2): 249-263.), which are incorporated herein by reference. A mutational catalogue summarising genomic rearrangements associated with a sample or group of samples may be referred to as a “rearrangement catalogue” or “rearrangement profile”. Methods for obtaining a rearrangement catalogue and deriving rearrangement signatures from such a catalogue are described in WO2017/191068A and in Nik-Zainal et al. (Nature. 2016 May 2; 534(7605): 47-54), both of which are incorporated herein by reference. This may comprise classifying rearrangements depending on whether they are present as “clustered” or “non-clustered” rearrangements, for example based on the density of rearrangement breakpoints in the region of rearrangement. This may comprise classifying rearrangements between the following classes: tandem duplications, deletions, inversion, translocations. As the skilled person understands, rearrangements are of a substantially larger size than deletions in the context of indels. For example, rearrangements may have a size of at least 1 kb. By contrast, indels may have a size below 1 kb. Obtaining a rearrangement catalogue may further comprise classifying rearrangements according to size of the rearranged segment (such as e.g. using the following classes: 1-10 kb, 10 kb-100 kb, 100 kb-1 Mb, 1 Mb-10 Mb, more than 10 Mb). Although the examples below illustrate the methods described herein in the context of substitution signatures, the same concepts are applicable to other types of mutations, in particular indels and rearrangements.

The methods described herein relate at least in part to determining an indication of which of the mutational signatures in a catalogue is present in the sample. This may be based on exposure of the mutational signatures in the catalogue (also referred to as “mutational signature metrics”). Methods for determining the exposure to a mutational signature are known in the art (see e.g. Alexandrov et al., 2020; Degasperi et al., 2020; Fantini et al., 2020; Gehring et al., 2015). In particular, the determination of the exposure to one or more mutational signatures in a set (such as e.g. a first set as described herein, or a combined set comprising a first set and one or more additional signatures from a second set) may be performed by identifying the matrix E that satisfies C˜PE where C is a mutational catalogue for one or more samples for which exposure is to be determined, P is a signature matrix comprising the one or more mutational signatures for which exposure is to be determined, and E is an exposure matrix. The determination of the exposure to one or more mutational signatures in a first set of mutational signatures (or a combined set comprising a first set and one or more additional signatures from a second set) may be performed as described in Degasperi et al., 2020.

The determination of the similarity between two mutation profiles or mutational signatures may be performed by calculating the cosine similarity between the two mutation profiles or two mutational signatures. The cosine similarity between two mutation profiles can be calculated as:

$$sim ( S , M ) = \frac{S \cdot M}{‖ S ‖ ‖ M ‖}$$

where S and M are equally-sized vectors with nonnegative components being the respective mutation profiles (e.g. S being that of a sample and M that of a reference profile such as e.g. a reconstructed profile) or mutational signatures.

A composition as described herein may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

As used herein “treatment” refers to reducing, alleviating or eliminating one or more symptoms of the disease which is being treated, relative to the symptoms prior to treatment.

The systems and methods described herein may be implemented in a computer system, in addition to the structural components and user interactions described. As used herein, the term “computer system” includes the hardware, software and data storage devices for embodying a system or carrying out a method according to the above described embodiments. For example, a computer system may comprise a processing unit such as a central processing unit (CPU) and/or graphical processing unit (GPU), input means, output means and data storage, which may be embodied as one or more connected computing devices. Preferably the computer system has a display or comprises a computing device that has a display to provide a visual output display. The data storage may comprise RAM, disk drives or other computer readable media. The computer system may include a plurality of computing devices connected by a network and able to communicate with each other over that network. It is explicitly envisaged that computer system may consist of or comprise a cloud computer.

The methods described herein may be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### Identifying Mutational Signatures in a Sample

In embodiments of the present invention, a characterisation of a DNA sample is performed in terms of the mutational signatures present in the sample. In these embodiments, this is performed by a computer-implemented method or tool that takes as its inputs sequence data from the sample and a mutational signatures catalogue comprising a first set of mutational signatures and a second set of mutational signatures, each set comprising one or more mutational signatures, and produces as output an indication of which of the mutational signatures in the catalogue is present in the sample (also referred to as “mutational signature metrics”).

In a development of this embodiment, the computer-implemented method or tool may take as its inputs a list of somatic mutations generated from sequence data associated with a tumour sample (such as e.g. sequencing data obtained from genomic material from fresh-frozen derived DNA, circulating tumour DNA or formalin-fixed paraffin-embedded (FFPE) DNA representative of a suspected or known tumour from a patient). These somatic mutations can then be analysed to determine the value(s) of the one or more mutational signature metrics. In a development of this embodiment, the computer-implemented method or tool may take as its inputs sequence data associated with a tumour sample, and may use this data to generate a list of somatic mutations. These somatic mutations can then be analysed to determine the value(s) of the one or more mutational signature metrics. A list of somatic mutation may be obtained by identifying mutations present in sequence data associated with a tumour sample, and removing or otherwise excluding mutations that are present or assumed to be present in a corresponding germline genome. Mutations that are present in a corresponding germline genome may be identified by identifying the mutations present in a germline sample obtained from the same subject (also referred to as a “matched germline” or “matched normal” sample). Thus, the computer-implemented method or tool may further take as input sequence data associated with a matched germline sample. Mutations that are assumed to be present in a corresponding germline genome may be identified by identifying mutations that are present in a reference genome or set of reference genomes. A reference genome or set of reference genomes may be obtained from one or more reference samples that are not (or not all) matched normal samples. For example, the reference sample(s) may be process matched, or may comprise a plurality of normal (i.e. non-tumour/non-modified) samples not all of which are matched to the sample for which a somatic mutational profile is determined (e.g. pooled normal samples may be used as references for a plurality of tumour samples). A reference genome or set of reference genomes may be obtained from one or more databases.

In a development of this embodiment, the computer-implemented method or tool may take as its inputs a mutational catalogue. In a development of this embodiment, the computer-implemented method or tool may take as its inputs at list of somatic mutations or sequence data associated with a tumour sample, and may use this data to generate a mutational catalogue.

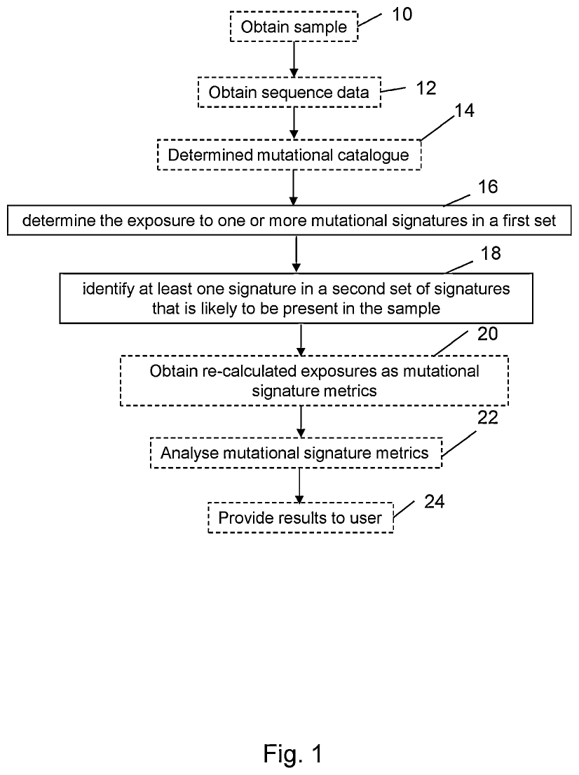

FIG. 1 is a flow diagram showing, in schematic form, a method of characterising a DNA sample according to the disclosure. At optional step 10, a DNA sample is obtained from a tumour of a subject. Optionally, a matched normal sample may also be obtained from the subject. At optional step 12, sequence data is obtained from the tumour (and optionally the matched normal) DNA sample(s). At optional step 14, a catalogue of somatic mutations in the tumour DNA is derived, for example by identifying somatic mutations in the tumour DNA and counting the number of mutations of a plurality of types (also referred to as “mutation channels” “classes” or “categories”). The types of mutations catalogued may comprise substitutions (single base substitutions, double base substitutions, triple base substitutions, etc.), deletions, insertions, and subsets (e.g. different trinucleotide substitutions, different lengths of indels, different indel contexts, etc.)/supersets (e.g. indels) thereof. The mutational catalogue is also referred to herein as “mutational profile”. Steps 10-14 are optional because the method may start from sequence data, or directly from a mutational profile associated with the sample. The mutational profile may then be used to determine the exposure to one or more mutational signatures in a first set at step 16. The results of step 16 may then be used at step 18 to identify at least one signature in a second set of signatures that is likely to be present in the sample. This may comprise identifying a signature that is most similar to the residual between the mutational profile of step 14 (observed mutational profile) and a reconstructed mutational profile obtained using the results of step 16 (i.e. signatures and their exposures identified in step 16). Alternatively, this may comprise identifying a signature that most reduces the error between observed and reconstructed mutational profile, when added to the first set of signatures. The process of step 18 may be repeated using the first set of signatures supplemented with a first signature (or set of signatures) of the second set of signatures identified in a first iteration of step 18. At step 20, a further set of exposures may be determined for the sample using the first set of signatures supplemented with any signatures of the second set of signatures identified in one or more iterations of step 18. At step 22, the final set of exposures may be used as mutational signature metrics, and analysed to identify for example mutational processes active in the sample, determined prognostic or therapeutic or diagnostic information using the mutational signature metrics, etc. This may comprise mapping the exposures for the signatures in the mutational catalogue to a reference set of signatures as described herein.

The method may further comprise receiving (for example from a user, through a user interface, or from one or more databases) one or more of: a first set of mutational signatures, a second set of mutational signatures, a set of reference signatures corresponding to the signatures in the first and/or second set, additional information associated with the sample such as e.g. known driver mutations, clinical information, etc. At step 24, one or more results of this analysis may optionally be provided to a user through a user interface.

### Uses of Analysis Outcome

A determination of the mutational signatures present in a sample can be used in identifying mutational processes that are or have been active in a sample. For example, mutational signatures have been associated with UV exposure, tobacco exposure, exposure to other mutagens, exposure to alkylating chemotherapy (e.g. platinum), mismatch repair (MMR) deficiency, homologous recombination (HR) deficiency, exposure to cytidine deaminase such as APOBEC, MBD4, POLE, MUTYH, OGG1, and NTHL1 related pathway deficiencies, etc. As the present invention provides methods by which mutational signatures present in a sample are identified with greater precision and certainty, they also provide methods to determine whether any of the mutational processes known to be associated with a mutational signature (such as e.g. those listed above and exemplified in the examples below or equivalent signatures obtained by signature extraction on a different cohort of samples) are present in a sample. Thus, the present disclosure also relates to methods of identifying mutational processes that are or have been active in a sample, using the methods described herein. Any of the mutational processes listed in Tables 12 and 13 may be considered to be present or to have been present in a sample if a mutational signature metric for a mutational signature associated with said process (such as eg. as provided in Tables 12 and 13) satisfies one or more predetermined criteria (e.g. minimum exposure, minimum exposure for an equivalent reference signature determined using e.g. a conversion matrix as provided in Tables 16 and 17 or an equivalent signature obtained by signature extraction on a different cohort of samples). Determination of the mutational signatures present in a sample thus provides important information that characterises a tumour. As such, exposures to one or more signatures obtained using the methods described herein, or metrics derived therefrom (such as scores from methods for determining whether a sample is from a tumour that has a characteristic that is indicative or prognosis or response to therapy) may be included in a report that characterises a tumour. This may be used for diagnostic purposes, for designing therapies, for selecting patients for a clinical trial, etc. Thus, also described herein are methods of determining whether a tumour has a characteristic that is indicative or prognosis or response to therapy, methods of characterising a tumour, methods of determining whether a tumour has a deficiency in a DNA repair pathway, comprising analysing a DNA sample from said tumour (whether the sample is directly obtained from the tumour or comprises genetic material from the tumour, such as ctDNA) or a mutational catalogue associated with said tumour using the methods described herein.

A determination of the mutational signatures present in a sample can be used to determine whether the DNA sample is from a tumour that has a characteristic that is indicative or prognosis or response to therapy. For example, deficiencies in some DNA repair pathways have been shown to be associated with different prognosis and/or different responses to particular courses of therapy. A determination of the mutational signatures present in a sample can be used to determine whether the sample is from a tumour that has a deficiency in a DNA repair pathway. For example, exposures to one or more signatures obtained using the methods described herein can be used as input to a method for determining whether the sample is from a tumour that has a deficiency in a DNA repair pathway. Examples of such methods are described below and in Davies et al. (Nature Medicine volume 23, pages 517-525 (2017)) and Zou et al. (Nat Cancer. 2021 June; 2(6): 643-657.). A determination of the mutational signatures present in a sample can be used in the treatment, management, diagnosing and prognosing of cancer. Indeed, various mutational signatures have been shown to be associated with treatment response and/or prognosis. Further, various mutational signatures have been shown to be indicative of the presence of mutational processes that sensitise or render a cancer resistant to a particular category of therapeutic approaches. For example, MMR deficiency has been shown to be indicative of response to immunotherapy, in particular checkpoint inhibitor therapy. CPI therapy includes for example treatment with an anti-CTL4 or anti-PD(L)1 drug. Thus, also described herein are methods of determining whether a subject that has been diagnosed as having a cancer is likely to benefit from treatment with an immunotherapy, preferably a CPI therapy, the method comprising determining the MMR status of a tumour from the subject using the methods described herein. The method may further comprise classifying the subject between a group that is likely to respond to CPI therapy, and a group that is not likely to respond to CPI therapy. For example, the method may comprise determining whether a sample from a tumour of the subject has a high or low likelihood of being MMR deficient, using at least one mutational signature metric identified as described herein (such as e.g. exposure to a signature that is associated with MMR deficiency, or a metric derived from said exposure—for example SBS6, SBS14, SBS15, SBS20, SBS26, SBS44, SBS97, DBS14, DBS19, DBS21, DBS28, DBS29, DBS33, DBS37 or an equivalent signature obtained by signature extraction on a different cohort of samples). In some cases CPI therapy may comprise CTLA-4 blockade (cytotoxic T-lymphocyte associated protein 4, Gene ID: 1493), PD-1 inhibition (PDCD1, programmed cell death 1, Gene ID: 5133), PD-L1 inhibition (CD274, CD274 molecule, Gene ID: 29126), Lag-3 (Lymphocyte activating 3; Gene ID: 3902) inhibition, Tim-3 (T cell immunoglobulin and mucin domain 3; Gene ID: 84868) inhibition, TIGIT (T cell immunoreceptor with Ig and ITIM domains; Gene ID: 201633) inhibition and/or BTLA (B and T lymphocyte associated; Gene ID: 151888) inhibition. The CPI therapy may be an anti-PD1 or anti-PDL1 therapy (also referred to as anti-PD(L)1 inhibitor). The inhibitor may be a therapeutic antibody. For example, the CPI therapy may be a PD-1 inhibitor such as pembrolizumab, nivolumab, or tislelizumab. Pembrolizumab is a therapeutic antibody that has been approved by the FDA (US Food and Drug Administration) for patients with unresectable or metastatic microsatellite instability-high (MSI-H) or mismatch repair deficient (dMMR) solid tumours that have progressed following prior treatment. This indication is independent of PD-L1 expression assessment, tissue type and tumour location. Nivolumab is a therapeutic antibody used to treat various cancers including melanoma, lung cancer, renal cell carcinoma, Hodgkin lymphoma, head and neck cancer, colon cancer, and liver cancer. Tislelizumab is a therapeutic antibody under investigation for the treatment of advanced solid tumours. The CPI therapy may be a PDL-1 (also referred to as “PD-L1”) inhibitor such as atezolizumab, avelumab, or durvalumab. Atezolizumab is a therapeutic antibody used to treat urothelial carcinoma, non-small cell lung cancer (NSCLC), triple-negative breast cancer (TNBC), small cell lung cancer (SCLC), and hepatocellular carcinoma (HCC). It was the first PD-L1 inhibitor approved by the FDA. Avelumab is a therapeutic antibody used for the treatment of Merkel cell carcinoma, urothelial carcinoma, and renal cell carcinoma. Durvalumab is a therapeutic antibody that has been approved by the FDA for the treatment of certain types of bladder and lung cancer. As another example, the CPI therapy may be a CTLA-4 inhibitor, such as ipilimumab or tremelimumab. Ipilimumab is a therapeutic antibody approved by the FDA for the treatment of melanoma, and under investigation for the treatment of non-small cell lung cancer, small cell lung cancer, bladder cancer and metastatic hormone-refractory prostate cancer. Tremelimumab is a therapeutic antibody under investigation for the treatment of melanoma, mesothelioma and non-small cell lung cancer. Similarly, MMR deficient cancers have been identified as having a decreased likelihood of response to fluorouracil based treatment (e.g. adjuvant 5-fluorouracil chemotherapy) and/or an increased likelihood of response to non-fluorouracil based treatments. Thus, also described herein are methods of determining whether a subject that has been diagnosed as having a cancer is likely to benefit from treatment with chemotherapy, preferably a fluorouracil based therapy or a non-fluorouracil based therapy, the method comprising determining the MMR status of a tumour from the subject using the methods described herein. Such a method may further comprise classifying the subject between a group that is likely to respond to fluorouracil based therapy, and a group that is not likely to respond to fluorouracil-based therapy. Additionally, the MMR status of a tumour has been shown to be associated with different prognosis in cancer (see e.g. Sinicrope, 2009). For example, MMR deficient tumours have been associated with improved prognosis compared to non-MMR deficient tumours, for example in terms of disease free survival and overall survival. Thus, also described herein are methods of providing a prognosis for a subject that has been diagnosed as having a cancer, the method comprising determining the MMR status of a tumour from the subject. The method may further comprise classifying the subject between a group that has good prognosis, and a group that has poor prognosis.

As another example, a subject may be identified as likely to be deficient for homologous recombination (HRdeficient) based at least in part on a mutational signature metric obtained using the methods described herein (e.g. a mutational signature metrics for a signature known to be associated with HR deficiency, such as e.g. SBS3 or an equivalent signature obtained by signature extraction on a different cohort of samples). This can be performed by using the mutational signature metric obtained using the methods described herein instead of a corresponding mutational signature metric obtained using one or more methods known in the art. Such a subject may be treated or identified as likely to benefit from treatment with a PARP inhibitor or platinum-based drug. For example, a subject may be identified as likely to be HR-deficient using the methods described in WO 2018/115452 or WO 2017/191074, or likely to respond to a PARP inhibitor or a platinum-based drug using the methods described in WO 2017/191073.

As another example, a subject may be identified as likely to be deficient for a pathway related to MBD4 based at least in part on a mutational signature metric obtained using the methods described herein (e.g. a mutational signature metrics for a signature known to be associated with MBD4 deficiency, such as e.g. SBS96 or an equivalent signature obtained by signature extraction on a different cohort of samples). This can be performed by using the mutational signature metric obtained using the methods described herein instead of a corresponding mutational signature metric obtained using one or more methods known in the art.

As another example, a subject may be identified as likely to be deficient for a pathway related to POLE based at least in part on a mutational signature metric obtained using the methods described herein (e.g. a mutational signature metrics for a signature known to be associated with POLE deficiency, such as e.g. SBS10a or an equivalent signature obtained by signature extraction on a different cohort of samples). This can be performed by using the mutational signature metric obtained using the methods described herein instead of a corresponding mutational signature metric obtained using one or more methods known in the art.

As another example, a subject may be identified as likely to be deficient for a pathway related to MUTYH based at least in part on a mutational signature metric obtained using the methods described herein (e.g. a mutational signature metrics for a signature known to be associated with MUTYH deficiency, such as e.g. SBS18 or an equivalent signature obtained by signature extraction on a different cohort of samples). This can be performed by using the mutational signature metric obtained using the methods described herein instead of a corresponding mutational signature metric obtained using one or more methods known in the art.

As another example, a subject may be identified as likely to be deficient for a pathway related to OGG1 based at least in part on a mutational signature metric obtained using the methods described herein (e.g. a mutational signature metrics for a signature known to be associated with OGG1 deficiency, such as e.g. SBS18 and/or SBS108 or an equivalent signature obtained by signature extraction on a different cohort of samples). This can be performed by using the mutational signature metric obtained using the methods described herein instead of a corresponding mutational signature metric obtained using one or more methods known in the art.

As another example, a subject may be identified as likely to be deficient for a pathway related to NTHL1 based at least in part on a mutational signature metric obtained using the methods described herein (e.g. a mutational signature metrics for a signature known to be associated with NTHL1 deficiency, such as e.g. SBS30 or an equivalent signature obtained by signature extraction on a different cohort of samples). This can be performed by using the mutational signature metric obtained using the methods described herein instead of a corresponding mutational signature metric obtained using one or more methods known in the art.

As another example, a subject may be identified as likely have a tumour that has DNA damage resulting from exposure to UV, tobacco, platinum or APOBEC based at least in part on a mutational signature metric obtained using the methods described herein (e.g. a mutational signature metrics for a signature known to be associated with UV, tobacco, platinum or APOBEC exposure, such as e.g. SBS7a for UV, SBS4 for tobacco, SBS31, SBS35, SBS111, SBS112 for platinum, SBS2, SBS13, SBS100 for APOBEC exposure, or an equivalent signature obtained by signature extraction on a different cohort of samples). This can be performed by using the mutational signature metric obtained using the methods described herein instead of a corresponding mutational signature metric obtained using one or more methods known in the art.

The subject may be a human patient. The subject may have been diagnosed as having or suspected of having a cancer. The cancer may be ovarian cancer, breast cancer, endometrial cancer (uterus/womb cancer), kidney cancer (renal cell), lung cancer (small cell, non-small cell and mesothelioma), brain cancer (gliomas, astrocytomas, glioblastomas), melanoma, merkel cell carcinoma, clear cell renal cell carcinoma (ccRCC), lymphoma, gastrointestinal cancer (e.g. colorectal cancer), small bowel cancers (duodenal and jejunal), leukemia, pancreatic cancer, hepatobiliary tumours, germ cell cancers, prostate cancer, head and neck cancers, bladder cancer, thyroid cancer and sarcomas. The cancer may be selected from biliary cancer, bladder cancer, cancer of the bones or soft tissues, breast cancer, central nervous system (CNS) cancer, colorectal cancer, esophagal cancer, head and neck cancer, kidney cancer, liver cancer, lung cancer, lymphoid cancer, myeloid cancer, neuroendocrine tumour (NET), oral or oropharyngeal cancer, ovarian cancer, pancreatic cancer, prostate cancer, skin cancer, stomach cancer, uterine cancer. The methods of the present disclosure have been demonstrated in each of these cancer types, although they are believed to be applicable to any cancer type. Mutational signature metrics for various mutational signatures have been demonstrated to be associated with different prognosis and/or treatment responses for example due to the indication of mutational processes present in a sample which are targeted by a particular treatment. Such information is more accurately obtained using the methods described herein, compared to the prior art. As such, the treatment strategy designed for a subject and/or the prognosis provided for a subject having cancer can be improved using the methods of the present invention.

Whether a prognosis is considered good or poor may vary between cancers and stage of disease. In general terms a good prognosis is one where the overall survival (OS), disease free survival (DFS) and/or progression-free survival (PFS) is longer than that of a comparative group or value, such as e.g. the average for that stage and cancer type. A prognosis may be considered poor if OS, DFS and/or PFS is lower than that of a comparative group or value, such as e.g. the average for that stage and type of cancer. Thus, in general terms, a “good prognosis” is one where survival (OS, DFS and/or PFS) and/or disease stage of an individual patient can be favourably compared to what is expected in a population of patients within a comparable disease setting. Similarly, a “poor prognosis” is one where survival (OS, DFS and/or PFS) of an individual patient is lower (or disease stage worse) than what is expected in a population of patients within a comparable disease setting.

### Systems

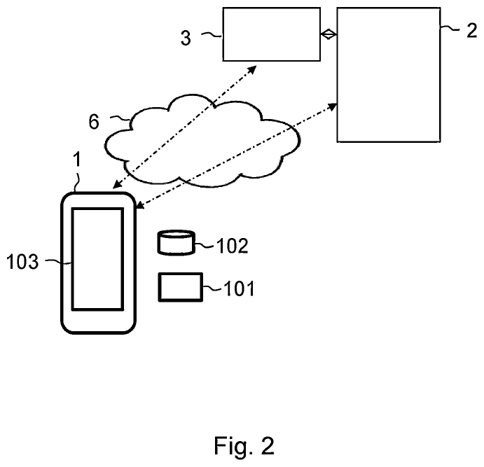

FIG. 2 shows an embodiment of a system for characterising a DNA sample and/or for providing a prognosis or treatment recommendation, according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 is communicably connected, such as e.g. through a network, to sequence data acquisition means 3, such as a sequencing machine, and/or to one or more databases 2 storing sequence data. The one or more databases 2 may further store one or more of: mutational signatures information (e.g. one or more mutational signature catalogues), clinical and/or sample related information, etc. The computing device may be a smartphone, tablet, personal computer or other computing device. The computing device is configured to implement a method for characterising a DNA sample, as described herein. In alternative embodiments, the computing device 1 is configured to communicate with a remote computing device (not shown), which is itself configured to implement a method of characterising a sample, as described herein. In such cases, the remote computing device may also be configured to send the result of the method of characterising a DNA sample to the computing device. Communication between the computing device 1 and the remote computing device may be through a wired or wireless connection, and may occur over a local or public network 6 such as e.g. over the public internet. The sequence data acquisition means may be in wired connection with the computing device 1, or may be able to communicate through a wireless connection, such as e.g. through WiFi and/or over the public internet, as illustrated. The connection between the computing device 1 and the sequence data acquisition means 3 may be direct or indirect (such as e.g. through a remote computer). The sequence data acquisition means 3 are configured to acquire sequence data from nucleic acid samples, for example genomic DNA samples extracted from cells and/or tissue samples. In some embodiments, the sample may have been subject to one or more preprocessing steps such as DNA purification, fragmentation, library preparation, target sequence capture (such as e.g. exon capture and/or panel sequence capture). Preferably, the sample has not been subject to amplification, or when it has been subject to amplification this was done in the presence of amplification bias controlling means such as e.g. using unique molecular identifiers. Any sample preparation process that is suitable for use in the determination of a genomic alteration profile (whether whole genome or sequence specific) may be used within the context of the present invention. The sequence data acquisition means is preferably a next generation sequencer.

The following is presented by way of example and is not to be construed as a limitation to the scope of the claims.

### EXAMPLES

Substantial efforts by The Cancer Genome Atlas (TCGA) (10), the International Cancer Genome Consortium (ICGC) (9, 11), and the Hartwig Medical Foundation (HMF) (12) have helped advance cancer genomics considerably in recent years. However, an endeavor to generate whole cancer genomes from national public health cancer services would be a welcome demonstration of how cancer genomic data can be derived from patients in real-time and ultimately benefit patients and the scientific community.

Here, we examined a new cohort of 15,838 WGS cancers from patients recruited from all 13 National Health Service (NHS) Genomic Medicine Centres across England as part of the Genomics England (GEL) 100,000 Genomes Project (100 kGP) (7, 13) [GEL v8 data release]. We report the analysis of mutational signatures and highlight a conceptual advance that come from being able to examine this substantial WGS collection. We add 40 single base substitution (SBS) mutational signatures and 18 double base substitution (DBS) mutational signatures to the current tally. We compare these additional signatures to known etiologies and end by suggesting principles of how to meaningfully utilize mutational signatures in future analyses.

### Example 1—Common and Rare Mutational Signatures

In this example, the inventors defined the concept of common and rare mutational signatures, identify a new signature catalogue from the GEL data and validate this catalogue and the common/rare signature approach by cross referencing with two independently extracted mutational signature catalogues.

### Results

The GEL cohort. All 15,838 tumor-normal sample pairs were taken through 100 kGP bioinformatic somatic-variant analysis pipelines. We restricted our analysis to high-quality data derived from flash frozen material, involving 12,222 GEL tumour samples from 11,585 individuals (several participants had synchronous or metachronous tumours). For this evaluation, the final dataset included 298,694,545 substitutions, 2,675,617 double substitutions, 154,675,475 indels, and 1,958, 105 rearrangements (Table 1) of 19 tumour types (skin, lung, stomach, colorectal, bladder, liver, uterus, ovary, biliary, kidney, pancreas, breast, prostate, bone/soft-tissue, central nervous system (CNS), lymphoid, oropharyngeal, neuroendocrine tumours (NET), and myeloid). This was complemented with data from the Hartwig Medical Foundation (HMF—3417 samples) and the ICGC (3001 samples).

TABLE 1
Count of somatic mutations in GEL, ICGC and Hartwig cohorts. Overall count of single
nucleotide variants (SNV), double nucleotide variants (DNV) small insertion and
deletions (Indels), and rearrangements (Rearr), across the GEL, ICGC and Hartwig
cohorts, along with the number of samples used. For the GEL cohort, we indicate
the proportion of samples that had PCR cycles in the library preparation.

| | mutation | | | | | PCRfree | PCRsamples |
|---|---|---|---|---|---|---|---|
| cohort | Type | count | median | samples | organs | Samples | Percentage |
| GEL | SNVs | 298,694,545 | 6,525.5 | 12,222 | 19 | 11,419 | 6.570119457 |
| | DNVs | 2,675,617 | 41.0 | 11,985 | 19 | 11,192 | 6.616604088 |
| | Indels | 154,675,475 | 904.0 | 12,222 | 19 | 11,419 | 6.570119457 |
| | Rearr | 1,958,105 | 86.0 | 11,985 | 19 | 11,192 | 6.616604088 |
| ICGC | SNVs | 47,630,580 | 5,162.0 | 3,001 | 19 | NA | NA |
| | DNVs | 429,910 | 24.0 | 3,000 | 19 | NA | NA |
| | Indels | 2,719,942 | 362.0 | 2,976 | 19 | NA | NA |
| | Rearr | 356,605 | 58.0 | 2,957 | 19 | NA | NA |
| Hartwig | SNVs | 91,624,434 | 11,376.0 | 3,417 | 18 | NA | NA |
| | DNVs | 1,196,381 | 74.0 | 3,417 | 18 | NA | NA |
| | Indels | 14,020,915 | 1,017.0 | 3,417 | 18 | NA | NA |
| | Rearr | 938,788 | 189.0 | 3,416 | 18 | NA | NA |

Common and rare mutational signatures. The national GEL sequencing endeavor delivers thousands of samples for certain tumor-types (1,009 lung, 1,355 kidney, 2,572 breast, and 1,480 bone/soft tissue cancers), an order of magnitude (or two) greater than previous WGS efforts for some organs. This permits robust detection of signatures that are rare—those occurring in 1% of the tumours or fewer. Furthermore, already-sequenced WGS cohorts such as ˜3,000 primary cancers from ICGC and ˜3,400 metastatic from HMF, provide a powerful means of validating findings.

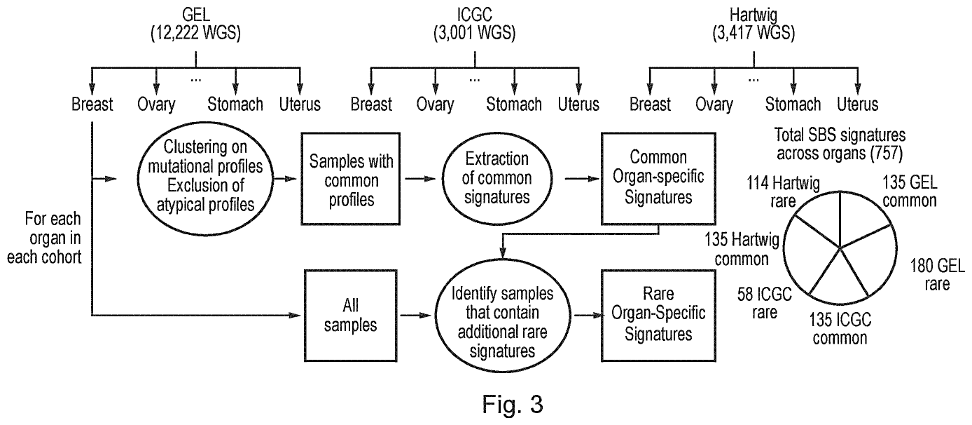

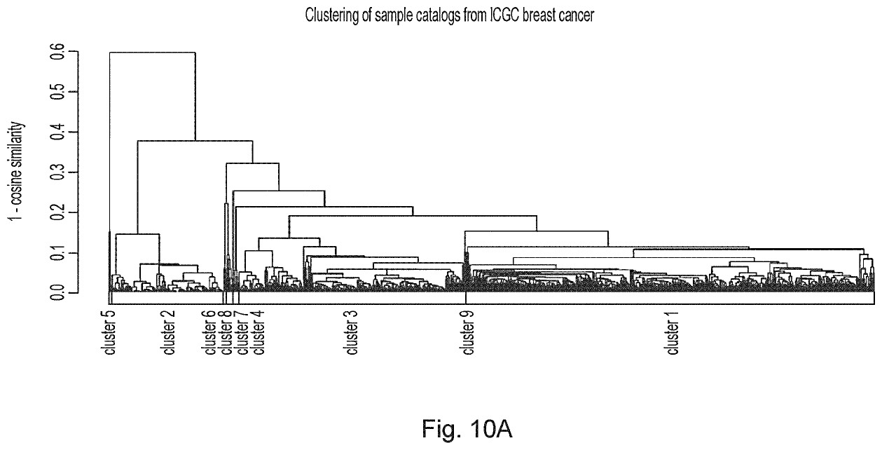

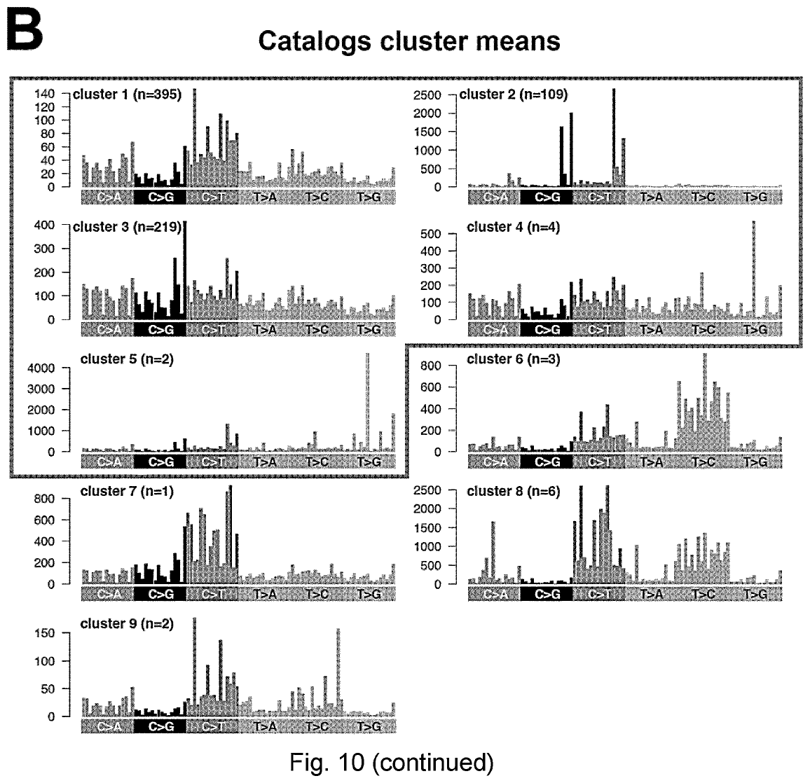

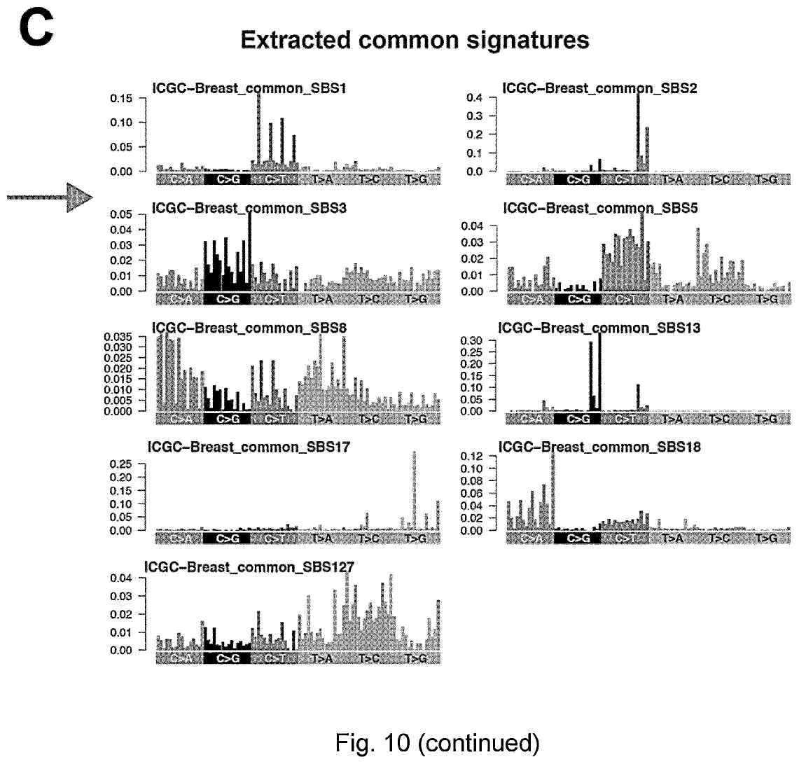

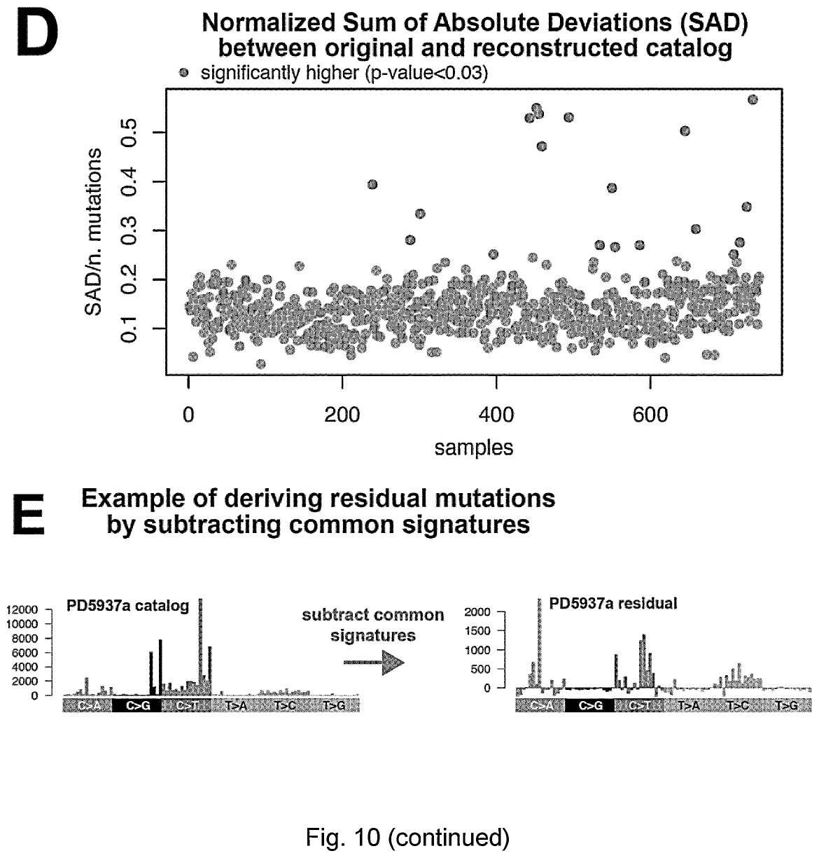

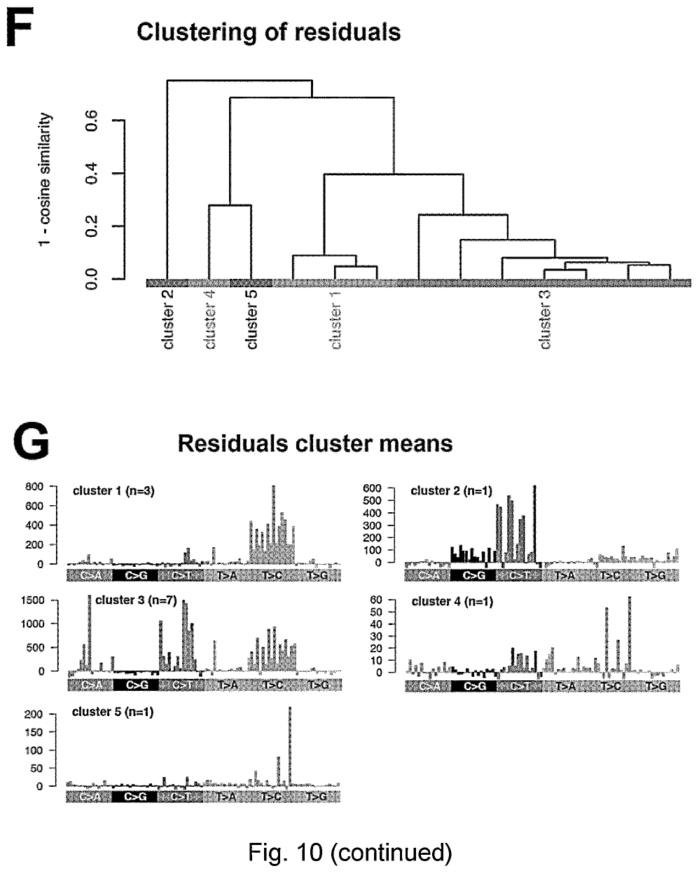

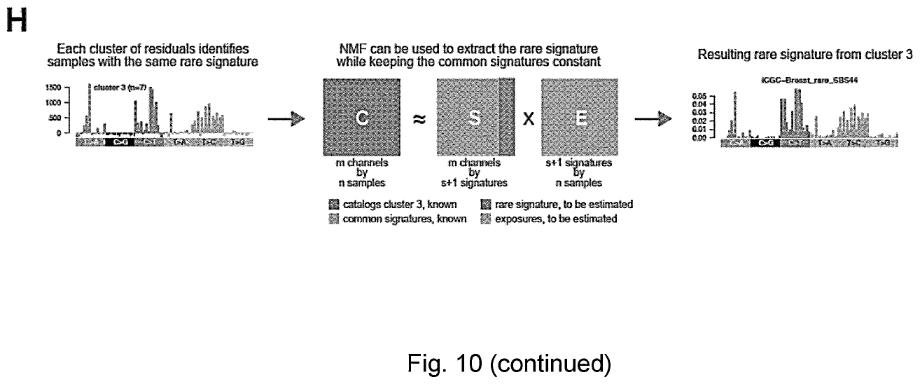

We performed mutational signature extractions confined to specific tumor-types using an updated signature extraction method (FIG. 3, FIG. 10, tables 2-3, Methods). Briefly, for each tumour type, we clustered mutational catalogues (counts of SBS in 96-element form or DBS in 78-element form), selecting only samples with recurrent, commonly occurring profiles to perform signature extraction (FIG. 10, A to C). Cases with unusual profiles and likely to have rare signatures were excluded in the first extraction. Thus, this yielded a set of highly accurate ‘common signatures’ that are prevalent for that tumour type. Next, by fitting these common signatures into all samples, cases that are likely to have additional patterns not fully explained by common signatures alone would report a high ‘error’ (or discrepancy between true sample catalogue and reconstructed catalog) (FIG. 10D). Potential additional signatures were then extracted from these samples to obtain a set of ‘rare signatures’ (FIG. 10, E to H, Materials and Methods). Accordingly, we obtained a set of common signatures and a set of rare signatures for each tumor-type. In all, for SBS, we identified 135 common signatures and 180 rare signatures in 19 tumour types within the GEL cancer cohort.

TABLE 2
Number of samples used for SBS analysis in
each cohort and in each organ.

| | GEL | ICGC | Hartwig |
|---|---|---|---|
| organ | samples | samples | samples |
| Biliary | 26 | 34 | 81 |
| Bladder | 349 | 23 | 140 |
| Bone_SoftTissue | 1480 | 89 | 164 |
| Breast | 2572 | 741 | 661 |
| CNS | 444 | 287 | 78 |
| Colorectal | 2348 | 52 | 493 |
| Esophagus | NA | 97 | 138 |
| Head_neck | NA | 56 | 61 |
| Kidney | 1355 | 186 | 100 |
| Liver | 21 | 314 | 48 |
| Lung | 1009 | 84 | 368 |
| Lymphoid | 181 | 197 | NA |
| Myeloid | 91 | NA | NA |
| NET | 92 | 80 | 100 |
| Oral_Oropharyngeal | 186 | NA | NA |
| Ovary | 523 | 110 | 144 |
| Pancreas | 59 | 233 | 75 |
| Prostate | 311 | 199 | 368 |
| Skin | 258 | 107 | 293 |
| Stomach | 204 | 68 | 39 |
| Uterus | 713 | 44 | 66 |
| Total in SBS analysis | 12222 | 3001 | 3417 |

TABLE 3
Number of samples used for DBS analysis in each
cohort and in each organ.

| | GEL | ICGC | Hartwig |
|---|---|---|---|
| organ | samples | samples | samples |
| Biliary | 15 | 27 | 76 |
| Bladder | 305 | 22 | 136 |
| Bone_SoftTissue | 970 | 50 | 108 |
| Breast | 1283 | 352 | 533 |
| CNS | 197 | 24 | 55 |
| Colorectal | 2300 | 50 | 487 |
| Esophagus | NA | 94 | 138 |
| Head_neck | NA | 43 | 47 |
| Kidney | 1251 | 152 | 98 |
| Liver | 21 | 307 | 47 |
| Lung | 944 | 77 | 360 |
| Lymphoid | 29 | 102 | 18 |
| NET | 41 | 18 | 62 |
| Oral_Oropharyngeal | 145 | NA | NA |
| Ovary | 415 | 103 | 129 |
| Pancreas | 36 | 149 | 64 |
| Prostate | 132 | 69 | 340 |
| Skin | 241 | 105 | 269 |
| Stomach | 190 | 58 | 37 |
| Uterus | 545 | 35 | 61 |
| Total in DBS analysis | 9060 | 1837 | 3065 |

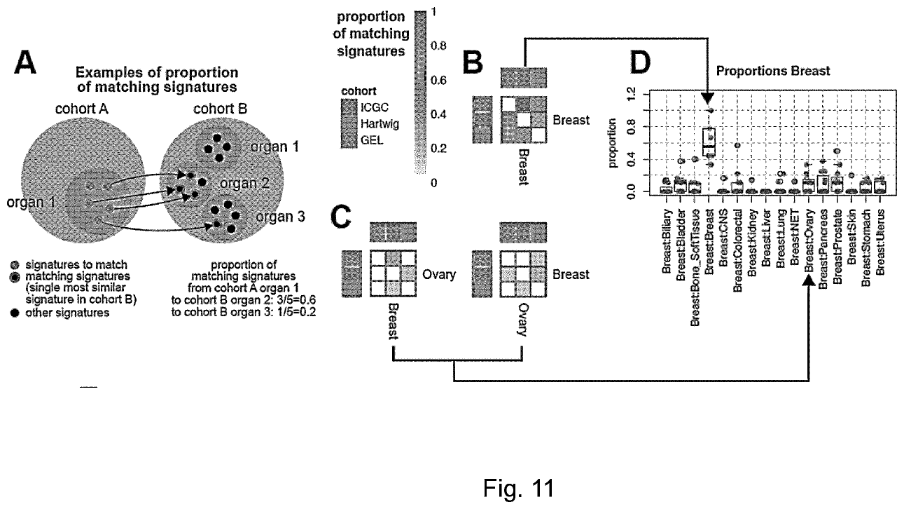

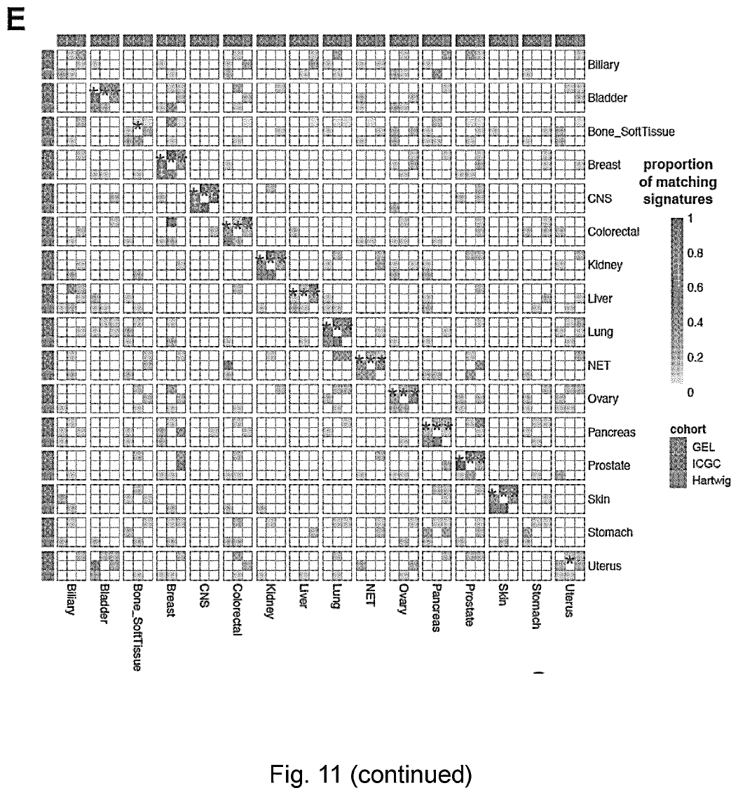

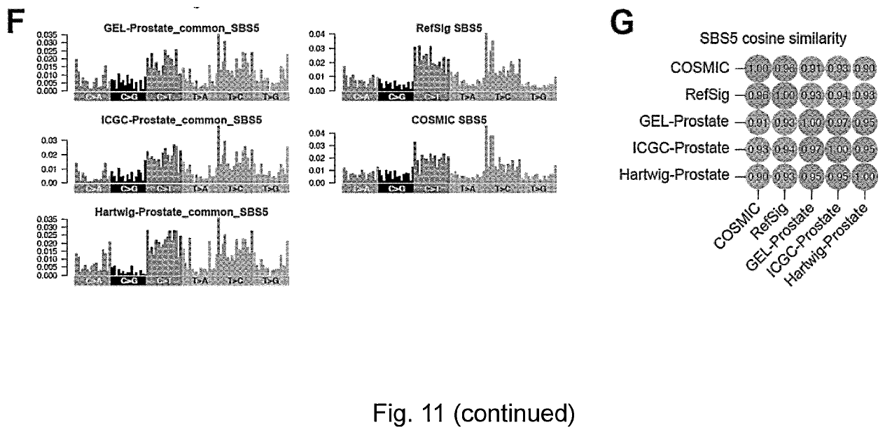

To validate these common and rare signatures, we performed signature extractions in independent cohorts of 3,001 ICGC primary WGS cancers (19 tumor-types) and 3,417 metastatic Hartwig WGS samples (18 tumor-types). We identified 135 common signatures in ICGC, 58 rare. In Hartwig, we found 135 common signatures and 114 rare. We performed an agnostic three-way signature comparison in 16 tissue types that were present in all three cohorts (FIG. 11). We found that signatures from the same organ in different cohorts were more similar to each other than to those in other tissue type, providing reassuring evidence that mutational signatures in each organ are highly reproducible, have tissue-specificities, and were detectable regardless of sequencing platform or mutation-calling algorithms (FIG. 11).

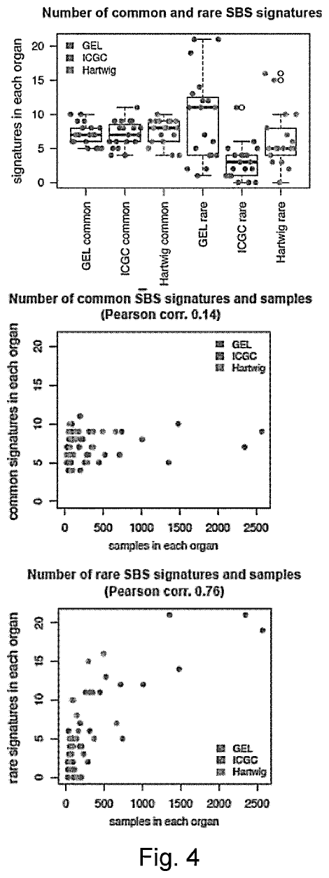

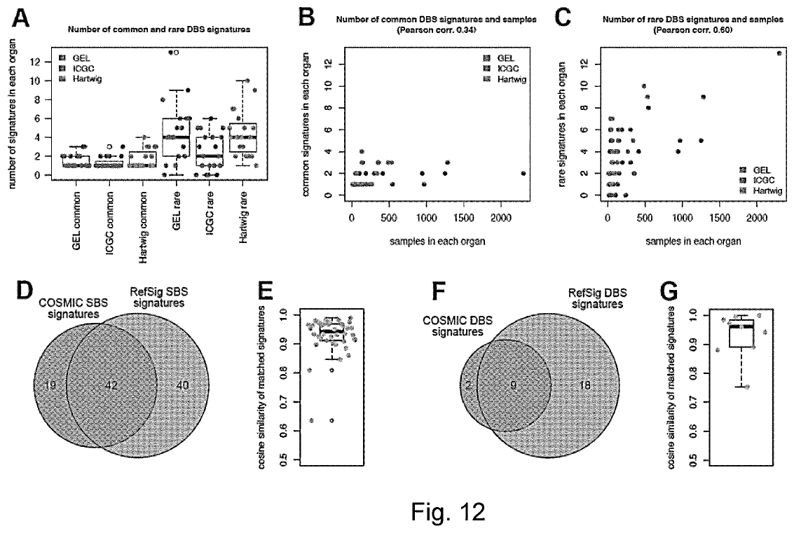

Notably, the number of common signatures in each organ is usually limited (between five and ten for SBS) and is independent of the number of samples analyzed per organ (FIG. 4, FIG. 12, A and B, tables 4 and 5). By contrast, the number of rare signatures varies and is highly correlated with the number of samples analyzed (FIG. 4 and FIG. 12C). This illuminates why ubiquitous, organ-specific signatures are detectable even with limited numbers of whole genomes, whereas sporadic, rare signatures are more likely to be detected with increased sample size.

TABLE 4
Number of common and rare SBS signatures extracted in each cohort and each organ.

| | GEL | GEL | GEL | ICGC | ICGC | ICGC | Hartwig | Hartwig | Hartwig |
|---|---|---|---|---|---|---|---|---|---|
| organ | Ncommon | Nrare | Ntot | Ncommon | Nrare | Ntot | Ncommon | Nrare | ntot |
| Biliary | 7 | 1 | 8 | 7 | 6 | 13 | 8 | 5 | 13 |
| Bladder | 7 | 11 | 18 | 6 | 0 | 6 | 8 | 8 | 16 |
| Bone_{—} | 10 | 14 | 24 | 8 | 2 | 10 | 9 | 6 | 15 |
| SoftTissue | | | | | | | | | |
| Breast | 9 | 19 | 28 | 9 | 5 | 14 | 9 | 7 | 16 |
| CNS | 5 | 11 | 16 | 6 | 2 | 8 | 4 | 10 | 14 |
| Colorectal | 7 | 21 | 28 | 5 | 4 | 9 | 9 | 16 | 25 |
| Esophagus | NA | NA | NA | 9 | 0 | 9 | 9 | 5 | 14 |
| Head_neck | NA | NA | NA | 8 | 4 | 12 | 10 | 5 | 15 |
| Kidney | 5 | 21 | 26 | 7 | 4 | 11 | 4 | 10 | 14 |
| Liver | 5 | 2 | 7 | 9 | 11 | 20 | 9 | 5 | 14 |
| Lung | 8 | 12 | 20 | 9 | 2 | 11 | 9 | 5 | 14 |
| Lymphoid | 9 | 4 | 13 | 11 | 0 | 11 | NA | NA | NA |
| Myeloid | 10 | 2 | 12 | NA | NA | NA | NA | NA | NA |
| NET | 8 | 5 | 13 | 5 | 5 | 10 | 6 | 4 | 10 |
| Oral | 7 | 7 | 14 | NA | NA | NA | NA | NA | NA |
| Oropharyngeal | | | | | | | | | |
| Ovary | 6 | 13 | 19 | 6 | 1 | 7 | 8 | 0 | 8 |
| Pancreas | 7 | 4 | 11 | 8 | 3 | 11 | 8 | 3 | 11 |
| Prostate | 6 | 6 | 12 | 4 | 4 | 8 | 7 | 5 | 12 |
| Skin | 5 | 11 | 16 | 8 | 1 | 9 | 5 | 15 | 20 |
| Stomach | 8 | 4 | 12 | 6 | 1 | 7 | 6 | 2 | 8 |
| Uterus | 6 | 12 | 18 | 4 | 3 | 7 | 7 | 3 | 10 |
| Total | 135 | 180 | 315 | 135 | 58 | 193 | 135 | 114 | 249 |

TABLE 5
Number of common and rare DBS signatures extracted in each cohort and each organ.

| | GEL. | GEL. | GEL | ICGC | ICGC. | ICGC | Hartwig | Hartwig | Hartwig |
|---|---|---|---|---|---|---|---|---|---|
| organ | ncommon | nrare | ntot | ncommon | nrare | ntot | ncommon | nrare | ntot |
| Biliary | 1 | 2 | 3 | 1 | 1 | 2 | 1 | 2 | 3 |
| Bladder | 2 | 6 | 8 | 1 | 1 | 2 | 4 | 4 | 8 |
| Bone_Soft | 1 | 5 | 6 | 1 | 0 | 1 | 1 | 4 | 5 |
| Tissue | | | | | | | | | |
| Breast | 3 | 9 | 12 | 3 | 2 | 5 | 3 | 9 | 12 |
| CNS | 1 | 3 | 4 | 1 | 1 | 2 | 1 | 3 | 4 |
| Colorectal | 2 | 13 | 15 | 1 | 4 | 5 | 3 | 10 | 13 |
| Esophagus | NA | NA | NA | 2 | 4 | 6 | 3 | 5 | 8 |
| Head_neck | NA | NA | NA | 1 | 5 | 6 | 1 | 6 | 7 |
| Kidney | 2 | 5 | 7 | 1 | 2 | 3 | 1 | 2 | 3 |
| Liver | 1 | 1 | 2 | 2 | 3 | 5 | 2 | 1 | 3 |
| Lung | 2 | 4 | 6 | 2 | 1 | 3 | 3 | 2 | 5 |
| Lymphoid | 1 | 4 | 5 | 2 | 0 | 2 | 1 | 2 | 3 |
| NET | 1 | 4 | 5 | 1 | 0 | 1 | 1 | 7 | 8 |
| Oral_{—} | 3 | 6 | 9 | NA | NA | NA | NA | NA | NA |
| Oropharyngeal | | | | | | | | | |
| Ovary | 2 | 4 | 6 | 1 | 4 | 5 | 1 | 3 | 4 |
| Pancreas | 1 | 2 | 3 | 1 | 5 | 6 | 1 | 4 | 5 |
| Prostate | 1 | 1 | 2 | 1 | 3 | 4 | 2 | 5 | 7 |
| Skin | 1 | 0 | 1 | 1 | 0 | 1 | 1 | 4 | 5 |
| Stomach | 2 | 6 | 8 | 1 | 5 | 6 | 1 | 7 | 8 |
| Uterus | 1 | 8 | 9 | 1 | 6 | 7 | 2 | 5 | 7 |
| Total | 28 | 83 | 111 | 25 | 47 | 72 | 33 | 85 | 118 |

### Methods

Datasets. We considered three large pan-cancer whole genome cohorts: the Genomics England Limited (GEL) version 8 cohort of the 100,000 Genomes Project (7) comprising 15,838 WGS paired samples, the ICGC cohort (9, 11) comprising 3,001 WGS paired samples, and the Hartwig cohort (12) of 3,417 WGS tumour samples. The GEL cohort contains 15,838 cancer whole genomes involving 23 tumor-types. We performed two steps of quality control: an automated check of sequencing and mapping quality parameters (see below), and a visual curation (e.g., missing data and evidence of contamination from other samples). We excluded formalin-fixed paraffin embedded samples (FFPE) and samples with short fragment size and low mapping rate. Around 6.5% of samples had a few cycles of PCR (table 1). The GEL version 8 dataset can be accessed via https://www.genomicsengland.co.uk/about-gecip/for-gecip-members/data-and-data-access. After considering comparability of tumor-types across cohorts and quality control (QC) of GEL data, we focused our analysis on 12,222 high quality WGS GEL cases (tables 1, 2, 3) across 19 organs. The filters used for quality control of GEL data were as follows:
preparation_method-=FF; FF is fresh frozen. Not used in liquid cancer samples
average_fragment_size>=300; minimum 300 bp
tumour_purity>=20; minimum 20%
tumour_sample_cross_contamination<1.0; less than 1%
coverage_homogeneity<=30; the lower the value the more homogeneous the coverage is
chimeric_percentage<=1; less equal than 1%
mapping_rate>=90; minimum 90%.

The ICGC cohort contains 3,001 cancer whole genomes across 19 organs, comprising 2471 samples from PCAWG (EGAS00001001692) and 530 additional breast cancers (450 from EGAS00001001178 and 80 from EGAD00001002740). The Hartwig cohort contains 3,417 metastatic cancer whole genomes across 18 organs. Data can be accessed via at www.hartwigmedicalfoundation.nl/en. The count of single nucleotide variants, double nucleotide variants, indels and rearrangements in the three cohorts can be found in table 1. The number of samples for each organ of each cohort can be found in tables 2 and 3.

Mutational Signature Extraction. For each tumour sample, we counted the number of somatic mutations and constructed SBS (96 channel) and DBS (78 channel) mutational catalogues (tables 9 and 10). Mutational signatures were analyzed independently for each tumour type in each of the three cohorts (FIG. 3). First, we clustered mutational catalogues and excluded samples with unusual profiles (hierarchical clustering using 1-cosine similarity as distance, average linkage) (FIG. 10, A to C), aiming at reducing the number of rare, complicating signatures and obtaining fewer, more accurate signatures. We then followed the dendrogram, and manually split the catalogues into two sets, one with the unusual/rare profiles and one with common/recurrent profiles, which was used as the initial set for the signature extraction (FIG. 10B). The dendrogram may instead be split automatically for example by excluding profiles that are in clusters below a predetermined size or profiles that are not within a predetermined distance of any other clusters (such as based on the average of clusters). The split of a dendrogram can be further checked by extracting signatures using the common profiles, fitting the signature catalogue to all profiles, identifying rare profiles that are adequately explained by the signatures extracted from the common profiles (based e.g. on the reconstruction error being below a predetermined value), and including those rare profiles in the set of common profiles to perform a new extraction of common signatures. From this initial set, we excluded GEL samples that were created using PCR library preparations, and samples with unusual/rare profiles. By excluding these samples, the number of mutational signatures in the initial set was limited to common patterns, reducing the mixing of signatures in the extraction process.

Second, we used non-negative matrix factorization (NMF) with Kullback-Leibler divergence (KLD) optimization, repeated bootstrapping (at least 300 bootstraps), and removed poor local minima (17). We identified a set of ‘common’ mutational signatures that were organ-and cohort-specific. In particular, given a matrix of catalogues C, we applied nonnegative matrix factorization (NMF) to 20 matrices C′, bootstrapped from C. To solve NMF we used the Lee and Seung multiplicative algorithm that optimizes the Kullback-Leibler divergence (KLD) (46), producing a matrix of signatures S and a matrix of exposures E for each NMF run, such that C′˜SE. We repeated NMF at least 300 times for each bootstrap matrix, using random initializations, and selected only the solutions that had a final KLD within 0.1% of the best solution found (the solution with the lowest KLD). Then, we clustered all the selected solutions using clustering with matching and computed the data-model error as the average KLD, the goodness of clustering as the average silhouette width (ASW), and the consensus signatures as the medoid of each cluster. Finally, we repeated the above procedure for different values of number of signatures k and manually selected k as the trade-off between data-model error and the ASW. Thus, for each organ in each cohort we reported a set of signatures, that we term common signatures.

Third, we fitted the common signatures into all samples of a given cohort and tissue type, and identified samples with high reconstruction error to identify unexplained processes or ‘rare’ mutational signatures (details in supplementary materials) (FIG. 10, D to H) (tables 18-19, 4-5). In particular, we fitted the common signatures S to all the samples (KLD optimization), and identified the samples with a high normalized error, calculated as the sum of absolute deviations between the original catalogue c and reconstructed catalogue Se, divided by the total mutations in the catalogue (FIG. 10D). In addition to the high normalized error, we also required that the samples had a residual error above a minimum number of mutations, which was manually tuned for each extraction (3-400 mutations for SBS and 40-50 mutations for DBS). A sample residual error (FIG. 10E) was calculated after estimating the sample exposures using least squares (limSolve R package) with the constraint that the difference between original and reconstructed catalogues should be mostly positive (c-Se>−τ·Σ!c!), τ=0.003. We then clustered the residual errors using hierarchical clustering with average linkage and 1-cosine similarity as distance (FIG. 10, F and G). Finally, for each cluster of residual errors, we extracted one signature using a version of NMF where the signature matrix S contained the common signatures as constants, and one additional column that was estimated to contain the new signature, using the NNLM R package, (FIG. 10H). Thus, for each organ in each cohort we reported an additional set of signatures, that we term rare signatures. The total number of common and rare SBS and DBS signatures found is 757 and 301 respectively (tables 18 and 19). The number of common and rare signatures found in each organ in each cohort can be found in tables 20 and 21. It should be noted that the terms common and rare refer to the step at which the signature was identified in a specific organ. In practice, a specific mutational pattern could be considered rare in one per organ extraction of one cohort and be a common pattern in another.

Mutational signature exposures. To define signature exposures in each sample, we used a signature ‘fit’ procedure. We fitted common and rare signatures to each sample catalogue independently. Rare signatures were fitted only into the samples where the signatures were identified. Briefly, the number of mutations attributed to each signature in each sample were estimated using organ-specific signatures detected in their originating cohort utilizing KLD optimization (NNLM R package) and bootstrapping (200 bootstraps) (17). Point estimates of exposures were the median of the exposures obtained from bootstrapping. Exposures below 5% of the total SBS burden or below 25% of DBS burden per sample were set to zero because of the risk of over-fitting. In particular, in the case of SBS signatures, as described previously (17), for each sample we performed 200 signature fits using bootstrapped catalogues and KLD optimization, obtaining an ensemble of 200 exposure estimates for each signature, and chose as a point estimate the median of the exposures. Finally, to increase specificity and reduce the false positive assignment we set to zero the point estimate exposure of a signature, if the proportion of exposures below a certain threshold (5% of the total number of mutations) was higher than 5% (empirical p-value of 0.05). Exposures of fewer than 50 mutations were also set to zero. In the case of DBSs, the number of mutations were too low to perform the bootstrap-based fit described above, so we performed a single signature fit instead. To increase specificity, exposures were set to zero if they contributed to less than 25% of the total number of mutations and if they were less than 25 mutations.

### Example 2—Reference Mutational Signatures

In this example, the inventors use the mutational signature catalogues extracted in Example 1 to identify a set of reference mutational signatures.

### Results

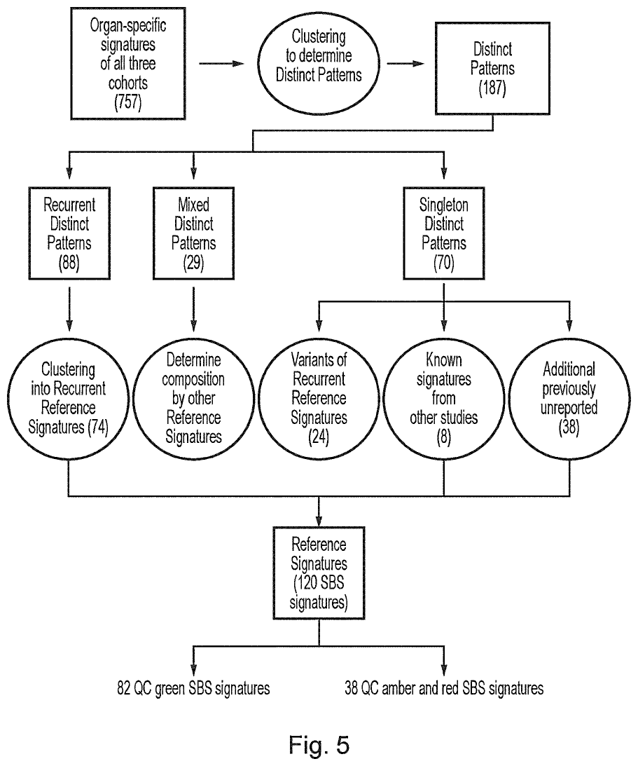

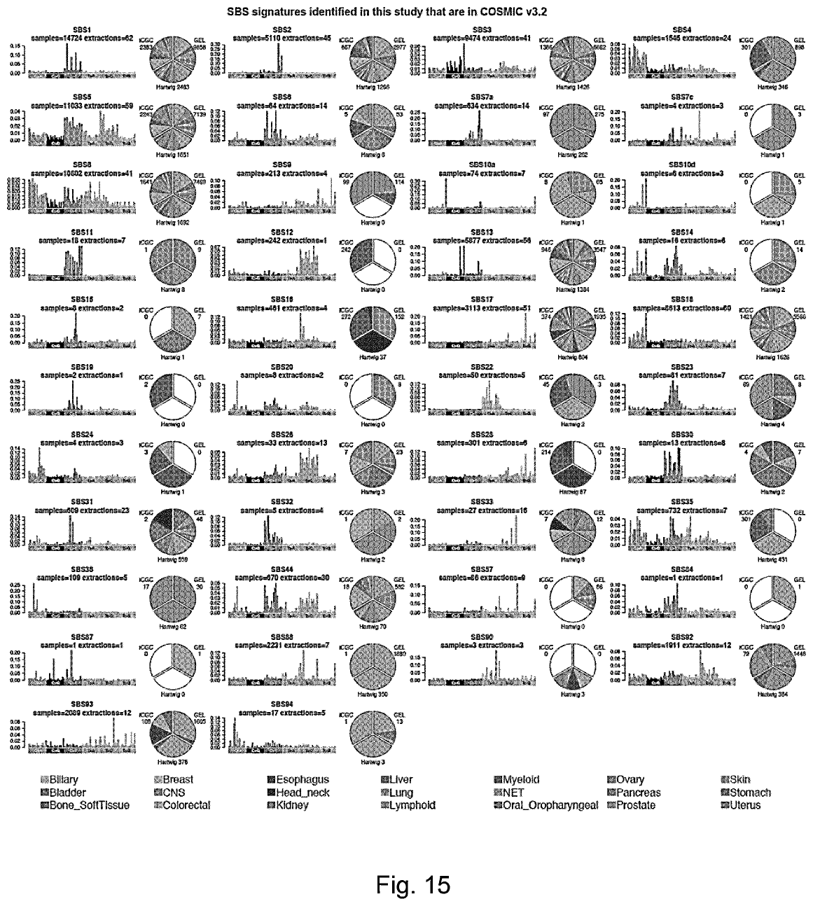

Biologically, the same mutational processes could underpin signatures extracted from different tumor-types. Thus, we considered all common and rare GEL, ICGC, and Hartwig tissue-specific signatures together, involving 18,640 WGS cancer samples (FIG. 5, Methods) and performed a clustering analysis to derive a set of ‘Reference Signatures’. First, we identified clusters of highly similar patterns that we termed ‘distinct patterns’ (tables 6 to 9). Each distinct pattern could be either: i) a true signature, thus observable in independent extractions of diverse organs and cohorts (recurrent pattern); ii) a mix of other signatures (mixed pattern); iii) a pattern seen in only one extraction (singleton pattern) (FIG. 5, tables 10 and 11). Next, we determined a minimal set of ‘Reference Signatures’, which were classified as quality control (QC) green, amber or red, where green implied high-confidence signatures observed in multiple independent extractions and amber/red signatures were observed only once or were possible mathematical artefacts (FIG. 5, tables 12-15). In all, we identified 82 SBS and 27 DBS high-quality signatures (FIGS. 15-17). Henceforth, we will only discuss these high-quality QC green signatures, although all signatures are available for reference in supplementary material (tables 12 and 13).

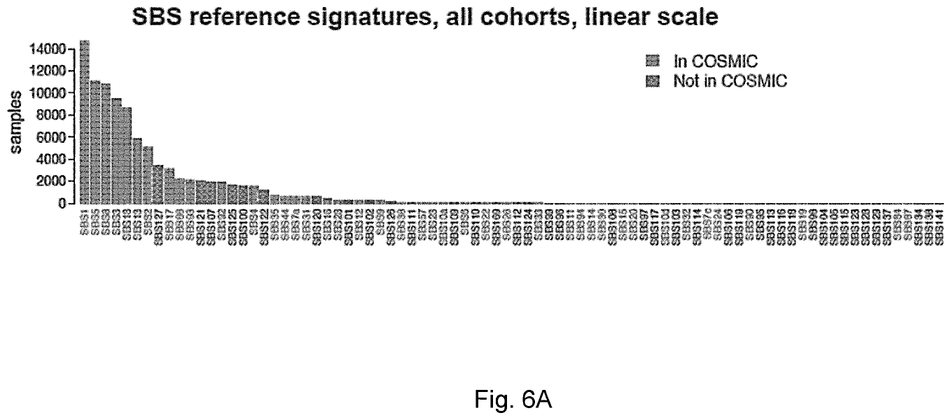

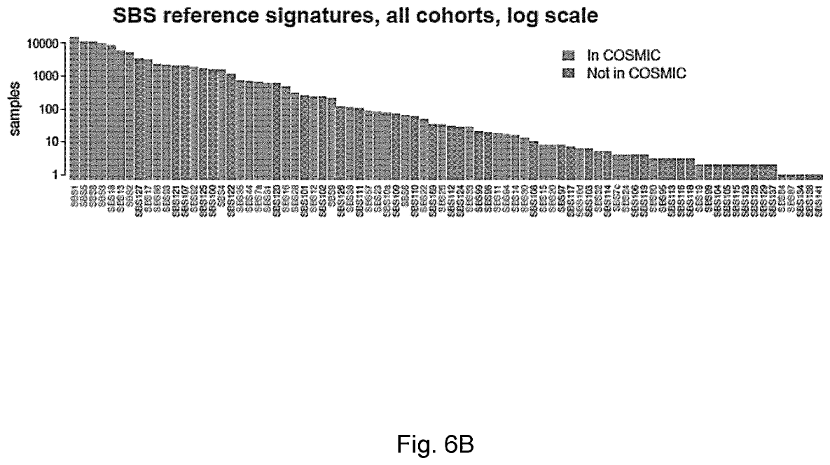

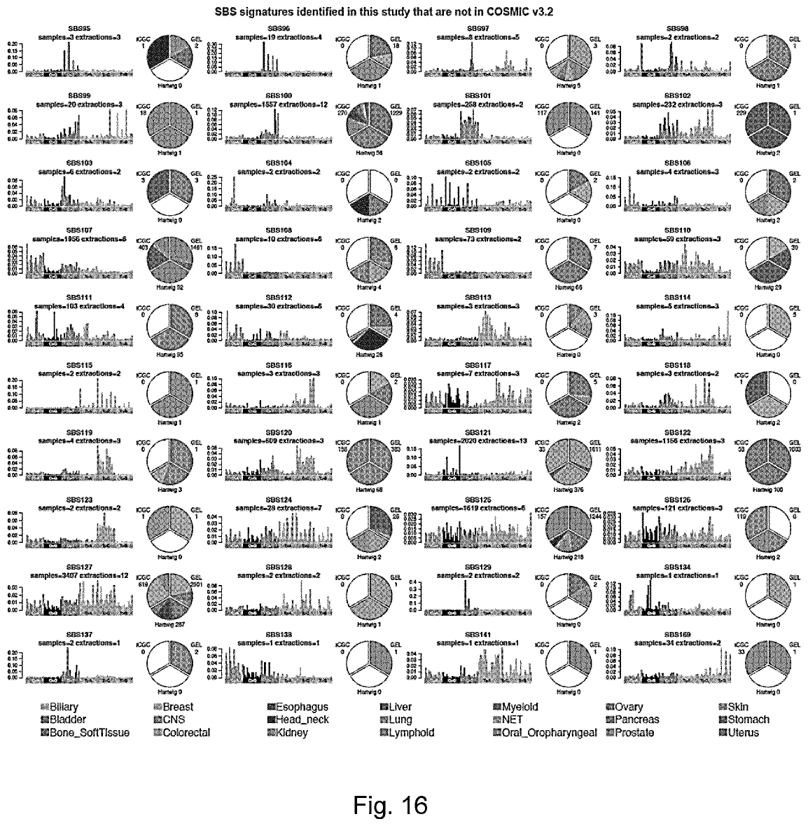

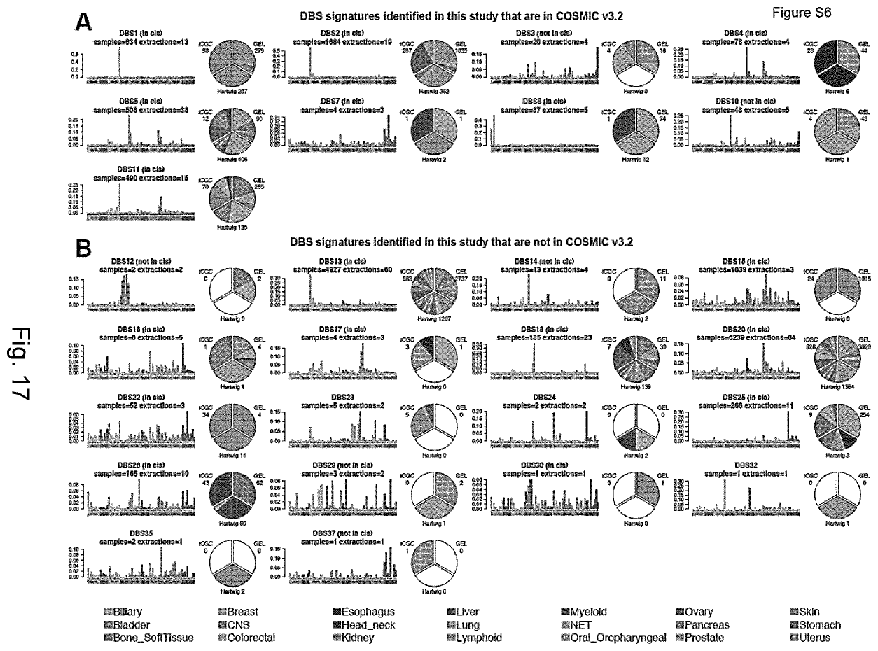

Reference Signatures were compared and matched with COSMIC mutational signatures (14), confirming 42 previously described COSMIC SBS signatures and 9 COSMIC DBS signatures (FIG. 6, A and B, FIG. 13, D to G, FIG. 15, FIG. 17A and table 12). We found 40 previously unreported high-confidence SBS signatures and 18 previously unreported DBS signatures in this analysis (FIG. 16 and FIG. 17B). Respecting prior nomenclature (14), these SBS signatures have been numbered from 95 onwards, and DBS signatures from 12 onwards (table 12). Note that COSMIC and/or Reference Signatures are a simplified means of discussing signatures that are mutually present across tissues. However, they are purely mathematical constructs—an averaged result across different organs—thus organ-specific signatures are more likely to be accurate biological representations of the mutational processes that occur within a tissue. We also provide matrices to map each reference signature to organ-specific signatures (tables 16 and 17), for SBS and DBS respectively.

Lengthy table referenced here
US20250191681A1-20250612-T00001
Please refer to the end of the specification for access instructions.

Lengthy table referenced here
US20250191681A1-20250612-T00002
Please refer to the end of the specification for access instructions.

Lengthy table referenced here
US20250191681A1-20250612-T00003
Please refer to the end of the specification for access instructions.

Lengthy table referenced here
US20250191681A1-20250612-T00004
Please refer to the end of the specification for access instructions.

Lengthy table referenced here
US20250191681A1-20250612-T00005
Please refer to the end of the specification for access instructions.

Lengthy table referenced here
US20250191681A1-20250612-T00006
Please refer to the end of the specification for access instructions.

Lengthy table referenced here
US20250191681A1-20250612-T00007
Please refer to the end of the specification for access instructions.

Lengthy table referenced here
US20250191681A1-20250612-T00008
Please refer to the end of the specification for access instructions.

Lengthy table referenced here
US20250191681A1-20250612-T00009
Please refer to the end of the specification for access instructions.

Lengthy table referenced here
US20250191681A1-20250612-T00010
Please refer to the end of the specification for access instructions.

Lengthy table referenced here
US20250191681A1-20250612-T00011
Please refer to the end of the specification for access instructions.

Lengthy table referenced here
US20250191681A1-20250612-T00012
Please refer to the end of the specification for access instructions.

### Methods

See Example 1.

Reference signatures. To permit comparability across cohorts and organs, we defined ‘reference signatures’ to denote unifying processes (FIG. 5). Each signature extracted in each cohort-organ combination could then be mapped to one or more reference signatures. In brief, we clustered all common and rare organ mutational signatures from all 3 cohorts (757 SBS or 301 DBS signatures) (tables 6 and 7) using hierarchical clustering with average linkage and 1-cosine similarity as distance. We then manually identified clusters by following the hierarchical clustering dendrogram (tables 6 and 7). Manual clustering was necessary because it was not possible to use a single threshold for the dendrogram that would be appropriate for all recurrent patterns. The clusters were selected so that all the signatures within each cluster were highly similar. We then computed the average of each cluster and termed these ‘distinct patterns’ (FIG. 5 and tables 8 and 9).

We obtained clusters of highly similar signatures (187 SBS and 60 DBS clusters). Cluster averages were termed ‘distinct patterns’ (tables 8 and 9). We assigned each distinct pattern to one of three groups: i) a reliably recurrent distinct pattern observed in multiple independent extractions; ii) a mix of two or more distinct patterns; iii) a singleton pattern found in one organ in one cohort (tables 10 and 11). We clustered the recurrent distinct patterns to determine whether some distinct patterns could be a variant of the same pattern. Cluster means were then reported as a first set of highly reliable reference signatures. Mixed distinct patterns that could be estimated as a combination of two distinct patterns using non-negative least squares were dismissed. To identify mixed distinct patterns, we performed a signature fit (KLD optimization) of each possible combination of two distinct patterns into each distinct pattern. Mixed patterns were not considered reference signatures, but rather a combination of reference signatures obtained from recurrent distinct patterns.

Singleton distinct patterns were also curated and dismissed if they could simply be variants of other reference signatures. If they had been reported in other studies, they were retained as reference signatures. A total of 120 SBS and 39 DBS reference signatures were identified. A conversion matrix was constructed to map the cohort-organ signatures to the reference signatures (tables 16 and 17). Most signatures can be mapped exactly to one reference signature (entry 1 in the conversion matrix) based on the distinct patterns clustering. Cohort organ signatures that clustered into mixed distinct patterns were mapped to multiple reference signatures using the coefficients determined at the identification of mixed distinct patterns. We used the conversion matrix and information about common/rare signatures to rename the cohort-organ signatures in a meaningful way. For example, “GEL-Ovary_common_SBS1+18” indicates that the signature is from the GEL cohort, Ovary organ, was identified among the common signatures, it is an SBS signature and according to the conversion matrix it is a mix of reference signatures SBS1 and SBS18. Finally, we used the conversion matrix to convert the cohort-organ signature exposures into reference signature exposures.

Quality control of reference signatures. A QC status was assigned to each of the reference signatures: green, amber or red, according to additional evidence. QC green signatures were those extracted independently multiple times and/or reported in orthogonal studies. QC amber status was given to signatures with limited supporting evidence, such as signatures identified in only one extraction and not reported previously. QC red status was assigned to signatures that were mathematical or alignment artefacts. After QC, 82/120 SBS and 27/39 DBS reference signatures remained QC green (tables 12 and 13, SBS/DBS final reference signatures (tables 14 and 15)). When seeking etiologies and/or potential artefacts for the signatures, we performed the following additional QC: (i) Genetically: we check relatedness of samples (because some patients do have more than one sample in the 100,000 Genomes Project); we seek potential germline variants as a contributing cause for a signature and; we go through somatic driver mutations; (ii) In many cases, medical records were searched for: past medical histories; past occupational exposures and; past treatment histories.

Organ-specificity of signatures. For all common signatures in 16 organs that were mutually present across GEL, ICGC and Hartwig, we sought the most similar signature in another cohort (minimum cosine similarity of 0.85) and checked whether it belonged to the same organ. For each organ in each cohort, this resulted in a proportion of signatures that best matched signatures of the same organ in a different cohort (FIG. 11A). These proportions could be calculated in all cohort directions: from ICGC to GEL, GEL to ICGC, GEL to Hartwig, Hartwig to GEL, ICGC to Hartwig and Hartwig to ICGC, resulting in six proportions per organ (FIG. 11B). We calculated the proportion of signatures that matched different organs as well, for example the proportion of GEL-Breast common signatures that best matched ICGC-Ovarian signatures, resulting in 12 proportion values for each match of different organs (FIG. 11C). Finally, we used a Tukey test (confidence level 0.95, p-value threshold 0.05) to determine whether the proportion of signatures matching the same organ was significantly higher than the proportion of signatures matching different organs (FIG. 11D). The total number of organ comparisons was 16, thus we counted how many times a comparison with the same organ was found to be significantly higher than the different organs comparisons, according to the Tukey test. In FIG. 11E, we simply reported *** for significantly higher than all other 15 organ comparisons, ** for higher than 11 other organ comparisons and * for higher than 7 other organ comparisons. Notice that these proportions do not simply indicate organ signatures similarity across cohorts, but rather that looking across an entire other cohort the most similar signatures are in a given organ. If the largest proportions are consistently found in same organ comparisons, this in turn implies organ-specificity of signatures. In some organs, such as biliary and stomach, while there were similar signatures in the same organ across cohorts, these were not consistently the most similar when considering all organs in a cohort, and organ-specificity was not detected.

Additional evaluations of DBS reference signatures. We performed three additional evaluations of the DBS signatures. First, for each DBS reference signature we selected representative samples that had a high number of mutations (exposures) associated with that signature. Then we manually checked aligned reads at DNV locations to determine if the two substitutions that composed each DNV were in cis, i.e., on the same DNA molecule. Second, for each SBS reference signature that had an associated DBS reference signature (high correlation of SBS and DBS exposures), we performed an in-silico analysis, to determine whether the DBS could be explained simply by SNVs of that signature falling adjacent to each other by chance. For each SBS, we sampled 1 million SNV mutations randomly across the genome with the same trinucleotide context and proportion of mutation types defined by the SBS. We then constructed the in-silico DBS using the SNVs that fell next to each other. Third, for each DBS reference signature we selected representative samples that had a high number of mutations (exposures) associated with that signature. Then, we inspected the mutational context of DNVs, up to 10 bp 5-prime and 3-prime of each DNV.

### Example 3—Analysis of New Mutation Signature Catalogue

In this example, the inventors investigated in-depth the mutational signatures identified in Examples 1 and 2.

### Results

Previously unreported mutational signatures—SBS signatures. We note four previously unreported and five recently reported signatures (15,16,17), that are common, recurring in many samples of multiple tumor-types in all three cohorts (GEL, ICGC, and Hartwig), detectable because of the scale of this analysis (SBS92, SBS100, SBS121, SBS93, SBS107, SBS125, SBS94, SBS110, SBS127). Among the previously unreported signatures, SBS107 is dominated by C>A variants and reported consistently in kidney and bladder cancers, suggestive of an organ-specific process. SBS100 bears similarities to the APOBEC signature SBS2; however, it presents a taller TCC>TTC peak and additional context-independent C>T mutations. SBS110 has the tallest T>A peak at CIG>CAG, with contributions from T>C at ATA and AIG. The preponderance in the liver/biliary tract would suggest a compound that is likely cleared through the hepatobiliary system. SBS121 is characterized by C>G variants mostly at ACT and TCT contexts, shows replication strand bias and is mostly found in colorectal and stomach cancer. We also confirm the recently reported SBS92 (15), SBS93, SBS94 (16), SBS125, and SBS127 (RefSig N12 and N1 respectively (17)). Three signatures occurred frequently in specific tumor-types: SBS120 dominated by T>C mutations at AIN and a distinctive peak of C>T at GCG, seen in 75% of CNS cancers; SBS122 characterized by T>C mutations in general but primarily TIN, in 67% of sarcomas; and SBS101 defined by C>T variants, in 68% of lymphoid cancers. Thirty-one additional rare previously unreported signatures of high-confidence were present in ˜1% or fewer samples. We discuss several in detail in relevant sections below, and for brevity, tabulate the majority in table 12. Associated information such as transcriptional and replication strand asymmetries are included there. All mutational signatures data can also be browsed at our website, Signal: https://signal.mutationalsignatures.com/explore/study/6.

Previously unreported mutational signatures—DBS and triple base substitution signatures. We adopted similar principles to identify 39 DBSs, including 27 high-confidence ones (Methods, table 13 and FIG. 17). We performed three additional evaluations. First, we curated dinucleotides for each DBS signature in the GEL dataset to check that they were in cis. Second, for a DBS signature that was correlated with an SBS signature, an in-silico analysis assessing whether the DBS pattern could be expected given the SBS pattern was performed (Methods). Third, we investigated up to 10 nt of mutational context of relevant dinucleotides for each DBS signature. These assessments were critical in refuting several DBS signatures as being simply due to chance, described below.

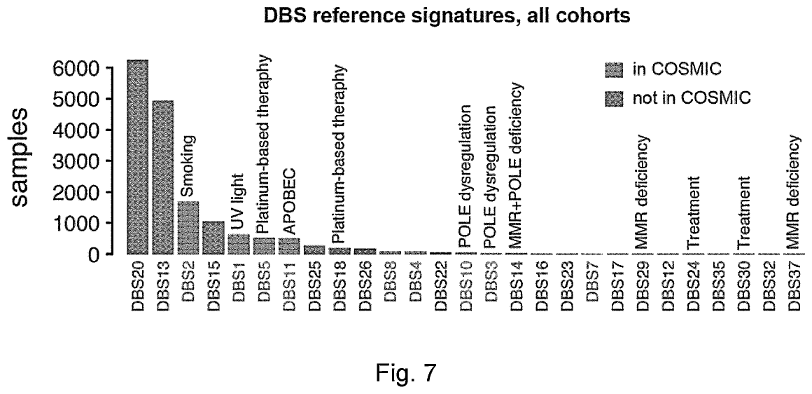

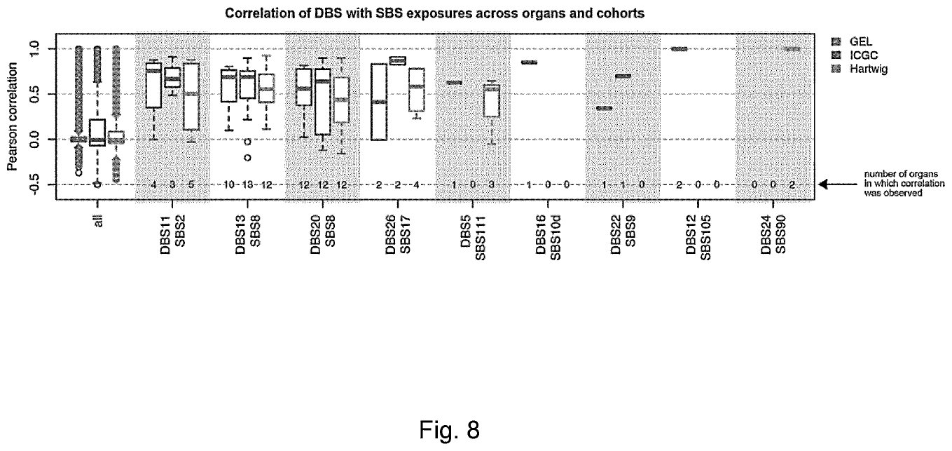

Of eleven previously described COSMIC DBS signatures (14), we identified nine and were unable to extract DBS6 or DBS9 (FIG. 7, FIG. 12, F and G, and FIG. S17A). Of our 27 high-confidence DBS signatures, 17 had bona fide dinucleotides in cis. We confirmed previously reported signatures and their associated etiologies: DBS1 (UV light), DBS2 (smoking), DBS5 (platinum therapy), and DBS11, associated with APOBEC, here verified as APOBEC-induced given the 10 nt sequence context analysis showing a TpCC preponderance (FIG. 3B). DBS7 was previously reported as associated with MMR defects (14), while we find associations with SBS17 instead. DBS8, mostly in colorectal cancer, showed dinucleotide variants often preceded by a Cytosine and followed by an Adenine. We confirm DBS5 and DBS18 are associated with prior platinum exposure (18). Mutational context analysis indicates that these are distinct signatures: DBS5 has the tallest peak of CT>AA mutations without preference in flanking sequences, while DBS18 has the tallest peak of CT>AC mutations, where the dinucleotide is always preceded by a Cytosine. Both signatures have a TG>GT peak most frequently followed by a Guanine. DBS13 and DBS20 were low-burden signatures that appear to correlate with each other and SBS8 (FIG. 8). DBS16 was associated with SBS10d (FIG. 8), a hypermutator signature recently reported as due to polymerase d (POLD) dysfunction (19). DBS22 is not associated with very prominent peaks (highest probabilities only 7%). However, it appears to be correlated with SBS9 and is only seen in lymphoid cancers (FIG. 8). DBS26 is similar to DBS7 and correlates with SBS17 in esophageal and stomach cancers (FIG. 8). DBS30 was observed in one lymphoid cancer sample and may be related to treatment (FIG. 17B). DBS25 is characterized by an excess of TT>AA that, on inspection, reveals a triple base substitution signature (TBS). Exploring all triple base possibilities, we obtain an 864-channel profile that systematically reports an excess of TTT>AAA followed by TTT>GAA, TTT>CAA, and minor contributions of TTG>AAA, and TTC>AAA. We propose that this is called TBS1. However, the number of mutations and implicated samples are too low to perform a formal mutational signature analysis.

Our curation steps uncovered several DBS signatures, including previously reported ones, that comprise adjacent substitutions that are not in cis and are simply the mathematical outcome of an associated SBS hypermutator. For example, DBS3 and DBS10 were similar and correlated with polymerase ε (POLE)-attributed SBS10a. In silico analysis showed that a DBS pattern that recapitulates DBS3/DBS10 could be reproduced from hypermutated samples of SBS10a. The alleged double substitutions were not, in fact, in cis. Similarly, DBS12 (associated with SBS105), DBS14 (associated with SBS14), DBS29 (associated with SBS20), and DBS37 (associated with SBS26) could all be generated mathematically from their associated SBS signatures, indicating that these were not true dinucleotides, but simply single nucleotide variants occurring next to each other by chance. One exception, DBS24-associated with SBS90, attributed to duocarmycin exposure-has a pattern that can be mostly recapitulated by simulation of SBS90, apart from the CT>AA component. Three signatures were not in the GEL cohort and could not be curated (DBS23, DBS32, DBS35) due to lack of access to sequencing data.

Contrasting Previously Unreported Signatures With Previously Reported Endogenous Processes Deamination and amplified deamination. Pervasive patterns of deamination are widely observed in malignant and non-malignant tissues. SBS1 characterized by C>T mutations at CpG is due to deamination of methyl-cytosine, while SBS2 and SBS13 are due to APOBEC-related deamination. Both are likely physiological: SBS1 occurs by natural hydrolytic processes, while SBS2 and SBS13 arise through transient single-stranded DNA availability (20). Two rare signatures also characterized by C>T transitions at CpG are SBS96 and SBS95, differing by their ability to demonstrate marked hypermutator phenotypes and relative C>T peak heights. SBS96, present in 18 of 12,222 GEL samples (0.15%, reported as due to inherited and/or acquired mutations in MBD4 (21), has C>T at ACG as its tallest peak. We identified germline truncating MBD4 mutations with loss of heterozygosity (LOH) of the alternative allele to explain 12 of 18 samples (6/10 patients) with SBS96. MBD4 germline variants were also seen in 35 other GEL patients, yet SBS96 was not observed in their tumours because the wild-type parental allele was intact in all assessable cases. We note that SBS96was observed in extremely rare cancers such as myxofibrosarcomas and uveal melanoma. SBS95 is distinguishable from SBS96 by having its tallest peak at CCG and by exhibiting transcriptional strand bias. SBS95 occurred in a lymphoid and a stomach cancer in GEL and one head and neck cancer in the ICGC cohort. None had MBD4 mutations. The cause for SBS95 remains unclear. Two signatures were characterized by C>N at CpG. SBS87 (22), with its tallest peak at CCG, was observed in one breast cancer. A related signature with C>N at all CpGs, SBS105, was reported in one breast and one bladder cancer in GEL. Although we have not found a cause for SBS105, it is associated with DBS12, a mathematical outcome of a high rate of SBS105, and does not exhibit transcriptional strand bias. Mechanistically, SBS105 would require deamination at CpGs followed by generic misincorporation during DNA replication and/or repair, not limited to the A-rule (23), to generate this pattern. Despite all occurring at CpGs, these signatures have distinguishing characteristics. Discriminating MBD4-related SBS96 is particularly important given reports that such tumours have sensitivities to checkpoint therapies (24).

DNA repair deficiency phenomena. A multitude of DNA repair genes and proteins serve as guardians of the genome (25). If compromised, they can result in mutational patterns in human cells. Compromised components of base excision repair (BER). SBS18 was previously described in neuroblastomas and adrenocortical cancers (5, 26). Subsequently, a hypermutated version of a signature similar to SBS18 was described in tumours from patients with biallelic mutations in MUTYH, a gene encoding a BER protein (MUTYH glycosylase) that corrects oxidative damage (27). Recently, it was demonstrated that OGG1 (8-oxo-guanine glycosylase) loss produces a phenocopy of SBS18 and that signatures defined by tall peaks at C>A at GCA, ACA, GCT, and TCT are due to an excess of 8-oxo-dG (25). Signature SBS108 resembles SBS18 and could be due to 8-oxo-dG (25) though has differences, including the tallest C>A peak at GCA instead of TCT. Intriguingly, three GEL patients having tumours with SBS108 all carried a germline polymorphism in OGG1 (rs113561019 p.G308E) that has been reported as a risk allele in microsatellite-stable hereditary nonpolyposis colorectal cancer (MSS-HNPCC) (28). We assessed the background frequency of this allele and found it present in 98 individuals (0.85%). Fifteen patients had tumours estimated as homozygous for the rs113561019 allele, including the three with SBS108 and 12 additional samples. It is possible that the presence of other strong signatures encumbered SBS108detection in these cases. Seven samples from six patients carried SBS30 associated with variants in NTHL1, another BER glycosylase. Two cases had germline nonsense NTHL1 mutations with associated loss of the wild-type parental allele. Three cases had somatic rearrangements deleting large sections of the gene. One of the three, GEL-2126555-11, an ovarian cancer, had a mixed phenotype of SBS30 and features of BRCA2 loss and carried a germline BRCA2 frameshift mutation which creates deletion signatures. This case also had two somatic deletions affecting NTHL1.

Mismatch repair and polymerase abnormalities. Replication of the nuclear genome occurs with high fidelity because of post-replicative mismatch repair (MMR) activity and base selectivity and proofreading capacity of DNA polymerases, particularly POLE and POLD. Unsurprisingly, MMR pathway defects and selected mutations in polymerases cause high rates of mutagenesis. We confirm four MMR deficiency (MMRd) signatures reported previously, including SBS6, SBS15, SBS26, and SBS44. As noted previously (5, 9, 14), we find a particular enrichment of mutations in MMR genes (MLH1, MSH2, and MSH6) in SBS6, SBS15, and SBS44, many of which exhibit loss of the alternative parental allele as well. In SBS26, previously shown to be identical to signatures of human knockouts of PMS2 (25), we indeed identified 14 PMS2 inactivating mutations (ten germline and four somatic, 7/14 biallelic) in 23 samples from 22 patients. Some caution should be exercised in interpreting somatic mutations in cancers with high burdens of substitutions or indels as these could be chance events. Regardless, it is worthy to note that a genetic driver cannot be identified for approximately one in every two cancers with MMRd signatures. Methylation data are not available for assessment. In addition, we confirm SBS10a is associated with POLE dysregulation. 100% of 65 GEL samples with SBS10a had POLE mutations consistent with proofreading dysfunction. We also confirm that two of five GEL samples with SBS10d carried POLD1 exonuclease domain mutation, p. (Asp316Asn) reported previously (29). Here, we report an identical p. (Asp903Tyr) mutation in DNA polymerase domain B in the remaining three samples. Two signatures were previously attributed to a mixed phenotype of MMRd and polymerase mutants, SBS14 (MMRd and POLE dysfunction) and SBS20 (MMRd and POLD dysfunction) (29). Of 14 samples with SBS14, 13 had potential POLE drivers (four established and nine putative, tables S29 and S30). Eleven out of fourteen samples also had truncating mutations in MMR genes (MSH6, MSH2, MLH1, or PMS2: three germline and 15 somatic mutations), but only six appeared to be inactivated on both parental alleles. Similarly, of eight samples with SBS20, four had missense drivers in POLD1 (one germline and four somatic). Seven of the eight also had inactivating mutations in MSH6 or MSH2, germline (n=4) and/or somatic (n=7), six of which showed biallelic inactivation. Again, all these tumours had high mutation burdens; thus, some mutations could be chance events due to high MMRd mutation rates. Moreover, elevated mutation rates of MMRd signatures cause a high likelihood of substitutions occurring adjacent to each other, falsely creating DBS patterns DBS14, DBS29, and DBS37. Lastly, we identify a signature with a defined C>T peak at GCG, SBS97, most closely resembling SBS15; however, it can be distinguished from SBS15 by strong T>C at GIC and T>G at GTT trinucleotides. Seen in three colorectal cancers in GEL and five in Hartwig, SBS97 is rare, has a strong hypermutator phenotype (29-65 subs/Mb), and a strikingly high indel rate exceeding substitutions (67-99 indels/Mb). All three GEL cases also have considerable structural variation (0.02-0.05 SV/Mb), revealing that chromosomal instability and microsatellite instability are not mutually exclusive in colorectal cancer. No causative drivers have been confirmed so far. In all, MMRd and polymerase-dysregulated signatures are prominent in colorectal (413/2,348, ˜18%) and uterine cancers (258/713, 36%) in the GEL cohort (FIG. 4D). Sporadic incidences of MMRd occurred in the stomach (11), prostate (3), pancreas (1), ovary (18), NET (2), lung (8), kidney (9), oropharyngeal (1), CNS (3), breast (14), sarcoma (16) and bladder cancers (3) (total 89/9,161, <1% total), with clinical implications.

Compromised components of double-strand break repair (DSBR). SBS3 was previously shown to distinguish BRCA1/BRCA2-null from sporadic breast cancers (6). SBS8 is increased in BRCA1/BRCA2-null cancers (9). We applied a previously developed algorithm, HRDetect (17, 30), designed to detect tumours with BRCA1/BRCA2-compromised DSBR, to the GEL cohort. The prevalence of HRDetect high scores (5th-95th percentile confidence interval above 0.5) was variable within each tumour type. More than 30% of all ovarian cancers had high HRDetect scores, ˜11% of breast cancers (predominantly estrogen receptor-positive cancers), ˜7% of pancreatic cancers, ˜4% of all uterine cancers, 1.6% of lung cancers, ˜1% of stomach cancer, and less than 1% of prostate, bone and colorectal cancers also had high scores. The causes of high HRDetect scores were identified in 231/493 individuals (47%, biallelic loss confirmed in 40%) and included germline and somatic mutations in BRCA1, BRCA2, PALB2, RAD51C, and RAD51D as described previously (6, 9, 31, 32). Promoter hypermethylation data were not available.

Environmental sources of mutational signatures: UV-like C>N signatures at CCN and TCN. We reinforce SBS7a (defined by C>T at CCN and TCN) in skin tumours with associated DBS1 characterized by CC>TT dinucleotides (33). However, we highlight three signatures that occurred at similar trinucleotides CCN/TCN and that could be miscalled as UV-related but may be due to alternative etiologies. SBS129, observed once in a nodular malignant melanoma (GEL-2501934-11) and once in a leiomyosarcoma (GEL-2300438-11), is characterized by C>T transitions at CCN, particularly CCA and CCT, but not TCN trinucleotides. It is distinguishable from SBS7a by its rarity and lack of CC>TT dinucleotides. However, SBS129 presents a transcriptional strand asymmetry with excess C>T mutations on the non-transcribed strand, the same as SBS7a. Apart from somatic TP53 mutations, no other potential genetic associations have been identified. SBS38 is identical in its trinucleotide preponderance to SBS129, except it results in C>A transversions instead. Although reported before (14), it is rare, and its etiology is unknown. Here, we identify it in 30 cancers (29 skin, one lung) in GEL and note that it can either be a dominating phenotype or occur in combination with SBS7a, SBS17, and SBS18. Notably, among the samples affected by SBS38, we found all three anorectal mucosal cancers in the GEL cohort, an aggressive, unusual mucosal melanocytic cancer. This uncommon signature occurring in a very rare tumor-type hints at a germline genetic predisposition. Yet, we have not been able to identify a causative gene. Minor transcriptional strand bias is noted with more mutations on the transcribed strand for C>A mutations. Lastly, SBS137 was observed twice in GEL brain cancers and would superficially seem highly similar to UV. Critically, affected tumours do not have a CC>TT DBS signature and demonstrate transcriptional strand bias in the opposite direction to UV, with an excess of C>T mutations on the transcribed strand (likely representing an excess of G>A on the non-transcribed strand). Its tallest peak is at CCC, dissimilar to the SBS7a peak at TCC. By contrast, in a metastatic CNS lesion derived from a cutaneous primary (GEL-2906789-11), the classic appearance of SBS7a and DBS1 is observed. This suggests that SBS137 is a distinct signature with currently uncertain cause.

Environmental sources of mutational signatures: Aristolochic-acid exposure and similar patterns. SBS22 is due to aristolochic acid (AAI) (33). All three renal cancers in GEL with SBS22 were from patients reporting ethnic-minority ancestry. None reported past exposure to AAI. We noted that SBS113 is similar to SBS22, has tall peaks in T>A with additional contributions from T>C at GIN, and is seen in one CNS (GEL-2585923-11), one colorectal (GEL-2282347-11), and one lung cancer (GEL-2158956-11). There is no history of exposure to AAI in these patients, although all three patients had complex therapeutic histories, including extensive exposure to psychotropic drugs and anti-epileptics. In previous work, alternative compounds from unrelated chemical families, specifically dibenzo [a, I] pyrene (DBP) and its diol-epoxide (DBPDE) from the polycyclic aromatic hydrocarbons (PAH) family in tobacco smoke, that caused bulky adducts on adenines similar to AAI, were capable of generating signatures nearly identical to AAI (33). Thus, given similarities to SBS22, SBS113 may represent mutational processes with alternative etiologies that also cause adducted adenines.

Environmental sources of mutational signatures: Platinum exposure. SBS31 is associated with prior platinum exposure (34) (FIG. 5D). This signature—characterized by C>T peaks at CCC and CCT, C>A peaks at ACC, CCT, GCC, and a modest T>A peak at CTN—has been demonstrated experimentally in a human cell line model previously (33). SBS35 is similar to SBS31, though it has smaller contributions at all trinucleotides and looks noisier (14). SBS104 may be related to SBS31 as it shows C>A peaks at CCC and CCT and was found in two Hartwig metastatic samples that had exposure to platinum. Two additional signatures, SBS111 and SBS112, have the components seen in SBS31, albeit with additional features particularly in C>A and noisier C>T components. Clinical histories of the patients carrying these signatures reveal that all had past diagnoses of primary malignant neoplasms of the ovary, stomach, esophageal cancer, breast and non-Hodgkin's lymphoma, and presented with secondaries or new primary malignancies. All patients had complex chemotherapy including platinum exposure. Perhaps these signatures are complex outcomes of multiple treatments and immune-modulation on the genome of the tumour samples isolated for sequencing. Two DBS platinum signatures (DBS5 and DBS18) are also associated with these SBS signatures.

Environmental sources of mutational signatures: Tobacco-related signatures and others with similar C>A components. SBS4, associated with tobacco smoke exposure (33), is seen mainly in lung cancers (at high levels ˜90 subs/Mb). SBS4 is noted very rarely in other tumor-types (table S23), including one breast cancer (GEL-2791664-11), one colorectal lesion noted to be ‘metastatic’ (GEL-2842602-11), one ‘diffuse astrocytoma’ (GEL-2645293-11), and two CNS lesions of unknown primary (GEL-2860373-11, GEL-2500813-11). SBS4 presence is supported by DBS2 and transcriptional strand bias in all these cases and probably indicates metastatic lesions of lung primary in these instances. Two signatures that have similarities to SBS4 are SBS94 and SBS109. SBS94 is characterized by C>A mutations with the tallest peak at CCC followed by CCA. In colon (9 cases) and breast (1 case), it does not have a hypermutator phenotype nor an associated DBS, but transcriptional and replication strand bias are noted for C>A variants (table S19). In bladder cancers (3 cases), there is a marked DBS pattern, despite low mutational burden (0.15-8 subs/Mb). The cause for this curious difference in tissue behavior is unclear. SBS109 is a C>A pattern with tall peaks at NCA and NCT, though tallest primarily at ACA and TCT. Only seven bladder cancers demonstrate this phenotype and it does not have any associated DBS or TSB. The mutation burden is also low at only 0.3-3 subs/Mb. SBS107 is seen at low levels in bladder and kidney cancers (0.04-6 subs/Mb) across many samples of these tumor-types. It is a common signature in kidney/bladder cancers (1,461/1,704) and is akin to SBS109 but with additional contributions at NCC. There are multiple signatures that have been attributed to environmental exposures which we will not discuss, including SBS11 (associated with alkylation on a mismatch repair deficient background), SBS90 (associated with duocarmycin), and SBS88 (reported as due to colibactin produced by pks+E. Coli infection) (35, 36).

### Methods

See Examples 1 and 2.

Replication and transcription strand bias were calculated as in previous work (42). Briefly, we counted classes of single nucleotide variants (C>A, C>G, C>T, T>A, T>C, T>G) taking into account whether they appeared on the lagging or leading strand (according to MCF-7 reference Repli-Seq data), or on the transcribed or non-transcribed strand (according to gene orientation) (42). A paired two-tailed Student's t-test was used to determine the significant deviation from the ‘natural’ bias given by the regions base content. The log2 ratio was used to determine the size of the asymmetry between the two strands.

HRDetect scores were computed as previously described (17, 30). HRDetect input features are exposures of SBS3 and SBS8, proportions of short deletions at microhomology, HRD-LOH index, and exposures of rearrangement signatures 3 and 5. Rearrangement signature exposures were estimated by using KLD optimization, bootstrapping, and previously published rearrangement signatures (17). HRDetect scores were computed both as point estimates and also as a distribution obtained from 1000 bootstrapped scores, as previously described (17).

Criteria for calling potential driver variants in GEL data. Potential driver mutations were sought in specific cancer genes associated with mutational signatures. For all genes investigated, germline variants which were called as pathogenic or likely pathogenic in ClinVar were included as potential drivers. For tumour suppressor genes any germline or somatic variant which was predicted to inactivate the gene was included as a potential driver variant. These included both substitutions and small insertions and deletions resulting in; stop gain, frameshift, splice donor and splice acceptor variants and structural rearrangement mutations (deletions, inversion, tandem duplications or translocations) which disrupted the footprint of the gene. In addition, for both tumour suppressor genes and oncogenes, somatic missense mutations which had been previously reported recurrently in cancer were also considered as potential drivers, including those variants recorded as pathogenic or likely pathogenic in

ClinVar and those present in COSMIC database greater than four times (https://cancer.sanger.ac.uk/cosmic). Additional published data was also used to assist driver assignment for the following genes, POLE (47), POLD1 (29) and MBD4 (21). Evidence to indicate all wild type alleles of tumour suppressor genes were inactivated in the tumour was provided by either the existence of two or more inactivating mutations or by Loss of Heterozygosity (LOH) of the alternate allele. LOH was indicated by a combination of copy number estimates provided by Canvas, tumour content and estimates of the Variant Allele Fraction (VAF) in the tumor. VAF was used to determine whether LOH of germline variants was in favor of the wildtype or mutant allele and in identifying variants with high VAF where LOH may have been missed by copy number analysis.

### Example 4—Utilising Mutational Signature Catalogues

In this example, the inventors provide a new approach to using mutational signature catalogues.

### Results

The ever-increasing number of mutational signatures poses the challenge of using mutational signature analysis in practice, whether in a new study of aggregated samples or for individual patients. To address this, we acknowledge that most non-expert users will aim to understand which mutational signatures are present in a new set of patient samples that are often tissue-specific. This signature ‘fitting’ process requires users to utilize a set of circumscribed signatures to ask which pre-defined signatures are present in their samples. To explore how to better perform fitting, we first consider mutational signatures per tumor-type, using CNS tumours from the GEL cohort as an example. Additional per tumour signature summaries can be found at our website, Signal: https://signal.mutationalsignatures.com/explore/study/6.

Per tumor-type summaries. A total of 809 WGS CNS tumours have been evaluated. Six percent of CNS tumours in GEL have rare signatures. Common signatures in the GEL CNS cohort that have been previously reported include age-associated SBS1 and SBS5, HR-deficiency-related SBS3 and SBS8, and a previously unreported common signature, SBS120,is present in many CNS tumours at a low to moderate mutation rate. Common CNS signatures exhibit clear and reproducible tissue-specificity. Rare signatures observed in the GEL CNS cohort that have been previously reported include the APOBEC signatures SBS2/SBS13, SBS17 of unknown etiology, SBS11 due to temozolomide on an MMR-deficient genetic background, and MMRd signatures (SBS14). We noted rare occurrences of tobacco-related SBS4 and UV-induced SBS7a in metastatic lesions. We also identified several previously unreported rare signatures in CNS tumours, including SBS113 mentioned earlier, with similarities to AAI-related SBS22. SBS121, defined by C>G at ACT and TCT, is common in colorectal and stomach cancers but seen in three CNS tumours only, and its etiology is unknown. SBS119 is present in a single CNS tumour as a hypermutator phenotype (28 SBS/Mb) in GEL and in two CNS tumours in Hartwig. Lastly, SBS137 is distinct from UV, has no DBS despite a high mutational burden, and is CNS-specific and rare. DBS1 and DBS2 are associated with UV and tobacco smoke exposure, respectively, and are seen in the samples with SBS7a and SBS4. Two previously unreported DBS signatures are observed: DBS13/DBS20 are relatively common, while DBS14 is due to the high mutational burden of MMRd SBS14. Reassuringly, common signatures are seen in all three cohorts (GEL, ICGC, and Hartwig) robustly, while the presence of rare signatures is a function of the size of the examined cohort. In all, this example highlights the landscape of common and rare signatures in this tumor-type and provides pointers regarding how to pragmatically use mutational signatures for signature fitting of new samples.

Fitting signatures: FitMS. Cancer samples have a median of five common signatures, and when rare signatures are present, there is usually only one existent per sample (FIG. 13, A and B). Learning from these results, we developed a signature-fitting algorithm, Fit Multi-Step (FitMS) (FIG. 13C), which first estimates the presence of tissue-relevant common signatures, and then attempts to identify additional rare signatures in a second step, assuming that only one rare signature or two may be present.

To evaluate the performance of FitMS, we performed a simulation study where each simulated sample comprised five organ-specific common signatures, and some samples carried one rare signature (Methods). We contrasted three strategies: first, fitting all common and rare signatures together in a single step (fit all); second, a two-step method fitting common signatures using a constraint of positive residuals that are matched to rare signatures in its second step (constrainedFit); and third, a two-step method fitting common signatures, followed by the addition of rare signatures one at a time to achieve a reduction in the residual between true and modeled catalogues (errorReduction). The two-step errorReduction FitMS strategy demonstrated superior performance (FIG. 13, D to F), improving the fit of common and rare signatures better than the ‘constrainedFit’ or ‘fit all’ approaches. Moreover, using organ-specific common signatures rather than corresponding reference signatures improved the accuracy of signature assignment (FIG. 13, G to I).

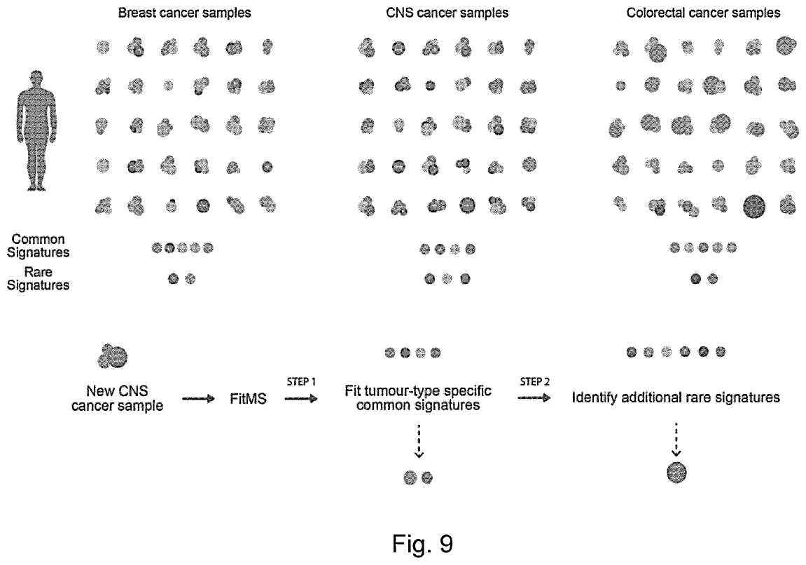

Therefore, for practical purposes, to assess which signatures are present in any new sample or set of samples, we recommend this two-step process (FIG. 9): first fitting common organ-specific signatures followed by a search for rare signatures, which can be achieved using FitMS.

### Methods

See Examples 1-3.

We provide a signature fitting algorithm called signature Fit Multi-Step (FitMS), which allows users to fit our mutational signatures into their own samples. FitMS is written in R and is available in our signature.tools.lib package (45).

Signature Fit Multi-Step (FitMS) is an algorithm designed to estimate signature exposures taking advantage of the concept of common and rare signatures. In general, given a mutational catalogue c, a signature fit algorithm attempts to find a set of nonnegative exposures e that indicate the number of mutations associated with each signature in a given signature matrix S, such that c˜Se. FitMS has two steps. In the first step, only common signature exposures are estimated. In the second step, the presence of potential rare signatures is estimated. In particular, the algorithm attempts to improve the fit by adding a small number of rare signatures (one by default). This is achievable through two possible strategies: constrainedFit or errorReduction. The constrainedFit strategy uses constrained non-negative least squares (limSolve R package) to estimate the residual between the observed and reconstructed catalogues, using only common signatures. If this residual resembled a rare signature (cosine similarity of at least 0.8) then we assumed that rare signature was present in the sample. In the errorReduction strategy, the error (KLD) between the original catalogue and the fit obtained using only common signatures was compared with the error obtained using one additional rare signature, for all rare signatures considered. A rare signature is considered present if the reduction in error is at least 15%.

Regardless of strategy, we recomputed sample exposures using both common signatures and any additional rare signatures. In particular, in the constrainedFit strategy: common signatures are fitted using a non-negative least squares algorithm with the additional constraint that the difference between the original catalogue c and the reconstructed catalogue Se should be mostly positive, c-Se>−τ·Σi ci, with Σ=0.003 (limSolve R package). The residual R=c-Se is then compared to the rare signatures, and if there are rare signatures with cosine similarity of at least 0.8 to R, then the rare signature with the highest cosine similarity is chosen. Finally, the common signatures and the selected rare signature are fitted into the catalogue using a non-negative KLD optimization (NNLM R package). In particular, in the errorReduction strategy: common signatures are fitted using a non-negative KLD optimization. All rare signatures are then fitted one at a time along with the common signatures. Rare signatures that caused the mean absolute deviation between c and Se to reduce at least 15% with respect to using the common signatures alone, are considered. Finally, if more than one rare signature is considered, the rare signature that induced the highest cosine similarity between the catalogue c and the model Pe is selected.

We determine the set of common and rare signatures to be fitted in a sample in an organ specific way. For common signatures, we use the GEL organ-specific common signatures, with the exception of Esophagus and Head_neck, where ICGC signatures are used, because these organs were not available in GEL. For rare signatures, we instead chose high-quality reference signatures observed as rare signatures across the various organs and cohorts at least twice, and that did not already belong to the set common signatures. The list of common and rare signatures that can be used in the 21 organs is available in table 20.

To evaluate the performance of FitMS (FIG. 13), we simulated 100 genomes, each containing 5 common signatures chosen randomly from the 9 GEL-Breast common SBS signatures. In addition, one rare signature was added to 25 out of 100 samples, each rare signature chosen randomly from 54 possible rare, curated SBS reference signatures observed in at least two independent extractions (Table 20). The minimum number of mutations in a sample was 5000 and maximum 50000, sampled uniformly in log scale, so that the values close to 5000 were more likely. We compared the two FitMS strategies against a “fit all” strategy, where all 9+54 signatures were used in one single signature fitting process. Each signature fit strategy produced a first estimate of the exposures, which tended to overfit signatures into samples, resulting in false positive assignments of signatures to samples with very few associated mutations. To remove false positives, we removed signature exposures that represented a very small proportion of mutations, testing thresholds from 0 to 10% of total sample mutations (FIG. 13, D to I).

TABLE 20
SBS common and rare signatures to be used with FitMS. Common and rare signatures are provided for each organ
analyzed in this study. Nc = n common. Nr = n rare.

| organ | common | rare (reference signatures—see Tables 12-15) | nc | nr |
|---|---|---|---|---|
| Biliary | GEL-Biliary_common_SBS18, | SBS3, SBS6, SBS8, SBS22, SBS24, SBS28, | 7 | 54 |
| | GEL-Biliary_common_SBS1, | SBS31, SBS32, SBS35, SBS44, SBS88, | | |
| | GEL-Biliary_common_SBS110, | SBS118, SBS4, SBS7a, SBS7c, SBS10a, | | |
| | GEL-Biliary_common_SBS13, | SBS10d, SBS11, SBS14, SBS15, SBS20, | | |
| | GEL-Biliary_common_SBS5, | SBS23, SBS26, SBS30, SBS33, SBS38, | | |
| | GEL-Biliary_common_SBS2, | SBS57, SBS90, SBS93, SBS94, SBS95, | | |
| | GEL-Biliary_common_SBS17 | SBS96, SBS97, SBS98, SBS99, SBS102, | | |
| | | SBS103, SBS104, SBS105, SBS106, | | |
| | | SBS108, SBS111, SBS112, SBS113, | | |
| | | SBS115, SBS116, SBS117, SBS119, | | |
| | | SBS121, SBS123, SBS124, SBS126, | | |
| | | SBS128, SBS129 | | |
| Bladder | GEL-Bladder_common_SBS13, | SBS4, SBS6, SBS15, SBS18, SBS20, | 7 | 58 |
| | GEL-Bladder_common_SBS17, | SBS23, SBS31, SBS33, SBS44, SBS57, | | |
| | GEL-Bladder_common_SBS92 + 100, | SBS88, SBS94, SBS104, SBS105, SBS109, | | |
| | GEL-Bladder_common_SBS1, | SBS125, SBS3, SBS5, SBS7a, SBS7c, | | |
| | GEL-Bladder_common_SBS2, | SBS8, SBS10a, SBS10d, SBS11, SBS14, | | |
| | GEL-Bladder_common_SBS2 + 13 + 107, | SBS22, SBS24, SBS26, SBS28, SBS30, | | |
| | GEL-Bladder_common_SBS92 | SBS32, SBS35, SBS38, SBS90, SBS93, | | |
| | | SBS95, SBS96, SBS97, SBS98, SBS99, | | |
| | | SBS102, SBS103, SBS106, SBS108, | | |
| | | SBS111, SBS112, SBS113, SBS115, | | |
| | | SBS116, SBS117, SBS118, SBS119, | | |
| | | SBS121, SBS123, SBS124, SBS126, | | |
| | | SBS128, SBS129 | | |
| Bone_ | GEL-Bone_SoftTissue_common_SBS127, | SBS2, SBS7a, SBS11, SBS26, SBS30, | 10 | 53 |
| SoftTissue | GEL-Bone_SoftTissue_common_SBS8, | SBS31, SBS44, SBS57, SBS93, SBS96, | | |
| | GEL-Bone_SoftTissue_common_SBS122, | SBS108, SBS117, SBS121, SBS124, | | |
| | GEL-Bone_SoftTissue_common_SBS1, | SBS129, SBS4, SBS6, SBS7c, SBS10a, | | |
| | GEL-Bone_SoftTissue_common_SBS100, | SBS10d, SBS14, SBS15, SBS20, SBS22, | | |
| | GEL-Bone_SoftTissue_common_SBS5, | SBS23, SBS24, SBS28, SBS32, SBS33, | | |
| | GEL-Bone_SoftTissue_common_SBS18, | SBS35, SBS38, SBS88, SBS90, SBS94, | | |
| | GEL-Bone_SoftTissue_common_SBS13, | SBS95, SBS97, SBS98, SBS99, SBS102, | | |
| | GEL-Bone_SoftTissue_common_SBS3 | SBS103, SBS104, SBS105, SBS106, | | |
| | GEL-Bone_SoftTissue_common_SBS17 | SBS111, SBS112, SBS113, SBS115, | | |
| | | SBS116, SBS118, SBS119, SBS123, | | |
| | | SBS126, SBS128 | | |
| Breast | GEL-Breast_common_SBS13, | SBS4, SBS6, SBS7a, SBS10d, SBS23, | 9 | 54 |
| | GEL-Breast_common_SBS5, | SBS26, SBS30, SBS31, SBS33, SBS44, | | |
| | GEL-Breast_common_SBS8, | SBS57, SBS87, SBS90, SBS94, SBS96, | | |
| | GEL-Breast_common_SBS2, | SBS97, SBS105, SBS106, SBS114, | | |
| | GEL-Breast_common_SBS3, | SBS116, SBS119, SBS123, SBS124, SBS7c, | | |
| | GEL-Breast_common_SBS18, | SBS10a, SBS11, SBS14, SBS15, SBS20, | | |
| | GEL-Breast_common_SBS1, | SBS22, SBS24, SBS28, SBS32, SBS35, | | |
| | GEL-Breast_common_SBS127, | SBS38, SBS88, SBS93, SBS95, SBS98, | | |
| | GEL-Breast_common_SBS17 | SBS99, SBS102, SBS103, SBS104, | | |
| | | SBS108, SBS111, SBS112, SBS113, | | |
| | | SBS115, SBS117, SBS118, SBS121, | | |
| | | SBS126, SBS128, SBS129 | | |
| CNS | GEL-CNS_common_SBS5, | SBS2, SBS4, SBS6, SBS7a, SBS11, | 5 | 57 |
| | GEL-CNS_common_SBS8, | SBS13, SBS14, SBS17, SBS18, SBS23, | | |
| | GEL-CNS_common_SBS120, | SBS26, SBS35, SBS97, SBS113, SBS119, | | |
| | GEL-CNS_common_SBS3, | SBS121, SBS126, SBS137, SBS7c, SBS10a, | | |
| | GEL-CNS_common_SBS1 | SBS10d, SBS15, SBS20, SBS22, SBS24, | | |
| | | SBS28, SBS30, SBS31, SBS32, SBS33, | | |
| | | SBS38, SBS44, SBS57, SBS88, SBS90, | | |
| | | SBS93, SBS94, SBS95, SBS96, SBS98, | | |
| | | SBS99, SBS102, SBS103, SBS104, | | |
| | | SBS105, SBS106, SBS108, SBS111, | | |
| | | SBS112, SBS115, SBS116, SBS117, | | |
| | | SBS118, SBS123, SBS124, SBS128, SBS129 | | |
| Colorectal | GEL-Colorectal_common_SBS17, | SBS4, SBS5, SBS6, SBS10a, SBS10d, | 7 | 53 |
| | GEL-Colorectal_common_SBS18, | SBS13, SBS14, SBS15, SBS22, SBS26, | | |
| | GEL-Colorectal_common_SBS93 + 121, | SBS30, SBS32, SBS33, SBS35, SBS44, | | |
| | GEL-Colorectal_common_SBS1, | SBS84, SBS94, SBS97, SBS108, SBS112, | | |
| | GEL-Colorectal_common_SBS88, | SBS113, SBS124, SBS128, SBS134, | | |
| | GEL-Colorectal_common_SBS3 + 8, | SBS7a, SBS7c, SBS11, SBS20, SBS23, | | |
| | GEL-Colorectal_common_SBS2 | SBS24, SBS28, SBS31, SBS38, SBS57, | | |
| | | SBS90, SBS95, SBS96, SBS98, SBS99, | | |
| | | SBS102, SBS103, SBS104, SBS105, | | |
| | | SBS106, SBS111, SBS115, SBS116, | | |
| | | SBS117, SBS118, SBS119, SBS123, | | |
| | | SBS126, SBS129 | | |
| Esophagus | ICGC-Esophagus_common_SBS2 + 13, | SBS4, SBS44, SBS90, SBS112, SBS127, | 9 | 52 |
| | ICGC-Esophagus_common_SBS28, | SBS5, SBS6, SBS7a, SBS7c, SBS10a, | | |
| | ICGC-Esophagus_common_SBS93, | SBS10d, SBS11, SBS14, SBS15, SBS20, | | |
| | ICGC-Esophagus_common_SBS18, | SBS22, SBS23, SBS24, SBS26, SBS30, | | |
| | ICGC-Esophagus_common_SBS17_a, | SBS31, SBS32, SBS33, SBS35, SBS38, | | |
| | ICGC-Esophagus_common_SBS100, | SBS57, SBS88, SBS94, SBS95, SBS96, | | |
| | ICGC-Esophagus_common_SBS1, | SBS97, SBS98, SBS99, SBS102, SBS103, | | |
| | ICGC-Esophagus_common_SBS17_b, | SBS104, SBS105, SBS106, SBS108, | | |
| | ICGC-Esophagus_common_SBS3 + 8 | SBS111, SBS113, SBS115, SBS116, | | |
| | | SBS117, SBS118, SBS119, SBS121, SBS123, | | |
| | | SBS124, SBS126, SBS128, SBS129 | | |
| Head_neck | ICGC-Head_neck_common_SBS1, | SBS4, SBS31, SBS33, SBS44, SBS88, | 8 | 54 |
| | ICGC-Head_neck_common_SBS18, | SBS95, SBS104, SBS111, SBS112, SBS121, | | |
| | ICGC-Head_neck_common_SBS17 + 18 + 100, | SBS125, SBS5, SBS6, SBS7a, SBS7c, | | |
| | ICGC-Head_neck_common_SBS2, | SBS10a, SBS10d, SBS11, SBS14, | | |
| | ICGC-Head_neck_common_SBS3 + 8, | SBS15, SBS20, SBS22, SBS23, SBS24, | | |
| | ICGC-Head_neck_common_SBS127, | SBS26, SBS28, SBS30, SBS32, SBS35, | | |
| | ICGC-Head_neck_common_SBS13, | SBS38, SBS57, SBS90, SBS93, SBS94, | | |
| | ICGC-Head_neck_common_SBS3 + 16 | SBS96, SBS97, SBS98, SBS99, SBS102, | | |
| | | SBS103, SBS105, SBS106, SBS108, | | |
| | | SBS113, SBS115, SBS116, SBS117, | | |
| | | SBS118, SBS119, SBS123, SBS124, | | |
| | | SBS126, SBS128, SBS129 | | |
| Kidney | GEL-Kidney_common_SBS13, | SBS2, SBS3, SBS6, SBS17, SBS18, | 5 | 61 |
| | GEL-Kidney_common_SBS5, | SBS22, SBS26, SBS31, SBS44, SBS57, | | |
| | GEL-Kidney_common_SBS1, | SBS90, SBS100, SBS108, SBS117, SBS121, | | |
| | GEL-Kidney_common_SBS125, | SBS126, SBS127, SBS138, SBS141, | | |
| | GEL-Kidney_common_SBS107 | SBS4, SBS7a, SBS7c, SBS8, SBS10a, | | |
| | | SBS10d, SBS11, SBS14, SBS15, SBS20, | | |
| | | SBS23, SBS24, SBS28, SBS30, SBS32, | | |
| | | SBS33, SBS35, SBS38, SBS88, SBS93, | | |
| | | SSB94, SBS95, SBS96, SBS97, SBS98, | | |
| | | SBS99, SBS102, SBS103, SBS104, SBS105, | | |
| | | SBS106, SBS111, SBS112, SBS113, | | |
| | | SBS115, SBS116, SBS118, SBS119, | | |
| | | SBS123, SBS124, SBS128, SBS129 | | |
| Liver | GEL-Liver_common_SBS5_a, | SBS1, SBS2, SBS3, SBS6, SBS8, SBS12, | 5 | 62 |
| | GEL-Liver_common_SBS4, | SBS13, SBS16, SBS18, SBS19, SBS22, | | |
| | GEL-Liver_common_SBS92, | SBS23, SBS24, SBS28, SBS30, SBS31, | | |
| | GEL-Liver_common_SBS5_b, | SBS35, SBS102, SBS103, SBS107, | | |
| | GEL-Liver_common_SBS110 | SBS118, SBS7a, SBS7c, SBS10a, SBS10d, | | |
| | | SBS11, SBS14, SBS15, SBS17, SBS20, | | |
| | | SBS26, SBS32, SBS33, SBS38, SBS44, | | |
| | | SBS57, SBS88, SBS90, SBS93, SBS94, | | |
| | | SBS95, SBS96, SBS97, SBS98, SBS99, | | |
| | | SBS104, SBS105, SBS106, SBS108, SBS111, | | |
| | | SBS112, SBS113, SBS115, SBS116, | | |
| | | SBS117, SBS119, SBS121, SBS123, | | |
| | | SBS124, SBS126, SBS128, SBS129 | | |
| Lung | GEL-Lung_common_SBS4_a, | SBS7a, SBS18, SBS28, SBS31, SBS33, | 8 | 52 |
| | GEL-Lung_common_SBS2, | SBS35, SBS38, SBS44, SBS113, SBS6, | | |
| | GEL-Lung_common_SBS3 + 8, | SBS7c, SBS10a, SBS10d, SBS11, SBS14, | | |
| | GEL-Lung_common_SBS92, | SBS15, SBS20, SBS22, SBS23, SBS24, | | |
| | GEL-Lung_common_SBS17, | SBS26, SBS30, SBS32, SBS57, SBS88, | | |
| | GEL-Lung_common_SBS13, | SBS90, SBS93, SBS94, SBS95, SBS96, | | |
| | GEL-Lung_common_SBS1 + 5, | SBS97, SBS98, SBS99, SBS102, SBS103, | | |
| | GEL-Lung_common_SBS4_b | SBS104, SBS105, SBS106, SBS108, | | |
| | | SBS111, SBS112, SBS115, SBS116, | | |
| | | SBS117, SBS118, SBS119, SBS121, | | |
| | | SBS123, SBS124, SBS126, SBS128, SBS129 | | |
| Lymphoid | GEL-Lymphoid_common_SBS1, | SBS3, SBS7a, SBS95, SBS100, SBS124, | 9 | 57 |
| | GEL-Lymphoid_common_SBS5_a, | SBS169, SBS2, SBS4, SBS6, SBS7c, | | |
| | GEL-Lymphoid_common_SBS18, | SBS10a, SBS10d, SBS11, SBS13, SBS14, | | |
| | GEL-Lymphoid_common_SBS9, | SBS15, SBS20, SBS22, SBS23, SBS24, | | |
| | GEL-Lymphoid_common_SBS127, | SBS26, SBS28, SBS30, SBS31, SBS32, | | |
| | GEL-Lymphoid_common_SBS101, | SBS33, SBS35, SBS38, SBS44, SBS57, | | |
| | GEL-Lymphoid_common_SBS5_b, | SBS88, SBS90, SBS93, SBS94, SBS96, | | |
| | GEL-Lymphoid_common_SBS5 + 8, | SBS97, SBS98, SBS99, SBS102, SBS103, | | |
| | GEL-Lymphoid_common_SBS17 | SBS104, SBS105, SBS106, SBS108, | | |
| | | SBS111, SBS112, SBS113, SBS115, SBS116, | | |
| | | SBS117, SBS118, SBS119, SBS121, | | |
| | | SBS123, SBS126, SBS128, SBS129 | | |
| Myeloid | GEL-Myeloid_common_SBS5_a, | SBS31, SBS57, SBS3, SBS4, SBS6, SBS7a, | 10 | 52 |
| | GEL-Myeloid_common_SBS1, | SBS7c, SBS10a, SBS10d, SBS11, | | |
| | GEL-Myeloid_common_SBS17, | SBS14, SBS15, SBS20, SBS22, SBS23, | | |
| | GEL-Myeloid_common_SBS18, | SBS24, SBS26, SBS28, SBS30, SBS32, | | |
| | GEL-Myeloid_common_SBS100, | SBS33, SBS35, SBS38, SBS44, SBS88, | | |
| | GEL-Myeloid_common_SBS8, | SBS90, SBS93, SBS94, SBS95, SBS96, | | |
| | GEL-Myeloid_common_SBS124, | SBS97, SBS98, SBS99, SBS102, SBS103, | | |
| | GEL-Myeloid_common_SBS5_b, | SBS104, SBS105, SBS106, SBS108, SBS111, | | |
| | GEL-Myeloid_common_SBS2 + 13, | SBS112, SBS113, SBS115, SBS116, | | |
| | GEL-Myeloid_common_SBS9 | SBS117, SBS118, SBS119, SBS121, | | |
| | | SBS123, SBS126, SBS128, SBS129 | | |
| NET | GEL-NET_common_SBS3 + 8, | SBS6, SBS7a, SBS11, SBS18, SBS23, | 8 | 53 |
| | GEL-NET_common_SBS4_a, | SBS44, SBS57, SBS93, SBS100, SBS121, | | |
| | GEL-NET_common_SBS4_b, | SBS123, SBS7c, SBS10a, SBS10d, SBS14, | | |
| | GEL-NET_common_SBS92, | SBS15, SBS20, SBS22, SBS24, SBS26, | | |
| | GEL-NET_common_SBS2 + 13, | SBS28, SBS30, SBS31, SBS32, SBS33, | | |
| | GEL-NET_common_SBS17, | SBS35, SBS38, SBS88, SBS90, SBS94, | | |
| | GEL-NET_common_SBS1, | SBS95, SBS96, SBS97, SBS98, SBS99, | | |
| | GEL-NET_common_SBS5 | SBS102, SBS103, SBS104, SBS105, | | |
| | | SBS106, SBS108, SBS111, SBS112, | | |
| | | SBS113, SBS115, SBS116, SBS117, | | |
| | | SBS118, SBS119, SBS124, SBS126, | | |
| | | SBS128, SBS129 | | |
| Oral_ | GEL-Oral_Oropharyngeal_common_SBS2, | SBS4, SBS7a, SBS10a, SBS31, SBS33, | 7 | 54 |
| Oropharyngeal | GEL-Oral_Oropharyngeal_common_SBS3 + 16, | SBS93, SBS121, SBS5, SBS6, SBS7c, | | |
| | GEL-Oral_Oropharyngeal_common_SBS17, | SBS8, SBS10d, SBS11, SBS14, SBS15, | | |
| | GEL-Oral_Oropharyngeal_common_SBS1, | SBS20, SBS22, SBS23, SBS24, SBS26, | | |
| | GEL-Oral_Oropharyngeal_common_SBS13, | SBS28, SBS30, SBS32, SBS35, SBS38, | | |
| | GEL-Oral_Oropharyngeal_common_SBS127, | SBS44, SBS57, SBS88, SBS90, SBS94, | | |
| | GEL-Oral_Oropharyngeal_common_SBS18 | SBS95, SBS96, SBS97, SBS98, SBS99, | | |
| | | SBS102, SBS103, SBS104, SBS105, | | |
| | | SBS106, SBS108, SBS111, SBS112, | | |
| | | SBS113, SBS115, SBS116, SBS117, | | |
| | | SBS118, SBS119, SBS123, SBS124, | | |
| | | SBS126, SBS128, SBS129 | | |
| Ovary | GEL-Ovary_common_SBS8, | SBS2, SBS6, SBS10a, SBS14, SBS26, | 6 | 53 |
| | GEL-Ovary_common_SBS3, | SBS30, SBS31, SBS35, SBS44, SBS57, | | |
| | GEL-Ovary_common_SBS17, | SBS103, SBS111, SBS116, SBS4, SBS7a, | | |
| | GEL-Ovary_common_SBS5, | SBS7c, SBS10d, SBS11, SBS15, SBS20, | | |
| | GEL-Ovary_common_SBS1 + 18, | SBS22, SBS23, SBS24, SBS28, SBS32, | | |
| | GEL- Ovary_common_SBS13 | SBS33, SBS38, SBS88, SBS90, SBS93, | | |
| | | SBS94, SBS95, SBS96, SBS97, SBS98, | | |
| | | SBS99, SBS102, SBS104, SBS105, | | |
| | | SBS106, SBS108, SBS112, SBS113, | | |
| | | SBS115, SBS117, SBS118, SBS119, | | |
| | | SBS121, SBS123, SBS124, SBS126, | | |
| | | SBS128, SBS129 | | |
| Pancreas | GEL-Pancreas_common_SBS1, | SBS6, SBS26, SBS28, SBS31, SBS44, | 7 | 54 |
| | GEL-Pancreas_common_SBS18, | SBS57, SBS98, SBS100, SBS108, SBS127, | | |
| | GEL-Pancreas_common_SBS5, | SBS4, SBS7a, SBS7c, SBS10a, SBS10d, | | |
| | GEL-Pancreas_common_SBS17, | SBS11, SBS14, SBS15, SBS20, SBS22, | | |
| | GEL-Pancreas_common_SBS1 + 5 + 18, | SBS23, SBS24, SBS30, SBS32, SBS33, | | |
| | GEL-Pancreas_common_SBS3 + 8, | SBS35, SBS38, SBS88, SBS90, SBS93, | | |
| | GEL-Pancreas_common_SBS2 + 13 | SBS94, SBS95, SBS96, SBS97, SBS99, | | |
| | | SBS102, SBS103, SBS104, SBS105, | | |
| | | SBS106, SBS111, SBS112, SBS113, | | |
| | | SBS115, SBS116, SBS117, SBS118, | | |
| | | SBS119, SBS121, SBS123, SBS124, | | |
| | | SBS126, SBS128, SBS129 | | |
| Prostate | GEL-Prostate_common_SBS2 + 13 + 92, | SBS4, SBS6, SBS32, SBS33, SBS35, | 6 | 52 |
| | GEL-Prostate_common_SBS17, | SBS44, SBS57, SBS93, SBS97, SBS116, | | |
| | GEL-Prostate_common_SBS1, | SBS121, SBS7a, SBS7c, SBS10a, SBS10d, | | |
| | GEL-Prostate_common_SBS5, | SBS11, SBS14, SBS15, SBS20, SBS22, | | |
| | GEL-Prostate_common_SBS18, | SBS23, SBS24, SBS26, SBS28, SBS30, | | |
| | GEL-Prostate_common_SBS3 + 8 | SBS31, SBS38, SBS88, SBS90, SBS94, | | |
| | | SBS95, SBS96, SBS98, SBS99, SBS102, | | |
| | | SBS103, SBS104, SBS105, SBS106, | | |
| | | SBS108, SBS111, SBS112, SBS113, | | |
| | | SBS115, SBS117, SBS118, SBS119, SBS123, | | |
| | | SBS124, SBS126, SBS128, SBS129 | | |
| Skin | GEL-Skin_common_SBS13, | SBS2, SBS3, SBS4, SBS6, SBS7c, SBS8, | 5 | 54 |
| | GEL-Skin_common_SBS1 + 5 + 18, | SBS23, SBS32, SBS33, SBS96, SBS99, | | |
| | GEL-Skin_common_SBS17, | SBS106, SBS112, SBS115, SBS124, | | |
| | GEL-Skin_common_SBS7a, | SBS125, SBS129, SBS10a, SBS10d, SBS11, | | |
| | GEL-Skin_common_SBS38 | SBS14, SBS15, SBS20, SBS22, SBS24, | | |
| | | SBS26, SBS28, SBS30, SBS31, SBS35, | | |
| | | SBS44, SBS57, SBS88, SBS90, SBS93, | | |
| | | SBS94, SBS95, SBS97, SBS98, SBS102, | | |
| | | SBS103, SBS104, SBS105, SBS108, | | |
| | | SBS111, SBS113, SBS116, SBS117, | | |
| | | SBS118, SBS119, SBS121, SBS123, | | |
| | | SBS126, SBS128 | | |
| Stomach | GEL-Stomach_common_SBS93 + 121, | SBS2, SBS26, SBS31, SBS44, SBS95, | 8 | 51 |
| | GEL-Stomach_common_SBS17, | SBS106, SBS112, SBS4, SBS6, SBS7a, | | |
| | GEL-Stomach_common_SBS13, | SBS7c, SBS10a, SBS10d, SBS11, SBS14, | | |
| | GEL-Stomach_common_SBS5, | SBS15, SBS20, SBS22, SBS23, SBS24, | | |
| | GEL-Stomach_common_SBS3 + 8, | SBS28, SBS30, SBS32, SBS33, SBS35, | | |
| | GEL-Stomach_common_SBS18 + 100, | SBS38, SBS57, SBS88, SBS90, SBS94, | | |
| | GEL-Stomach_common_SBS18, | SBS96, SBS97, SBS98, SBS99, SBS102, | | |
| | GEL-Stomach_common_SBS1 | SBS103, SBS104, SBS105, SBS108, | | |
| | | SBS111, SBS113, SBS115, SBS116, | | |
| | | SBS117, SBS118, SBS119, SBS123, | | |
| | | SBS124, SBS126, SBS128, SBS129 | | |
| Uterus | GEL-Uterus_common_SBS17, | SBS5, SBS9, SBS10a, SBS10d, SBS14, | 6 | 54 |
| | GEL-Uterus_common_SBS3 + 8, | SBS20, SBS26, SBS31, SBS44, SBS108, | | |
| | GEL-Uterus_common_SBS1, | SBS111, SBS4, SBS6, SBS7a, SBS7c, | | |
| | GEL-Uterus_common_SBS18, | SBS11, SBS15, SBS22, SBS23, SBS24, | | |
| | GEL-Uterus_common_SBS13, | SBS28, SBS30, SBS32, SBS33, SBS35, | | |
| | GEL-Uterus_common_SBS2 | SBS38, SBS57, SBS88, SBS90, SBS93, | | |
| | | SBS94, SBS95, SBS96, SBS97, SBS98, | | |
| | | SBS99, SBS102, SBS103, SBS104, SBS105, | | |
| | | SBS106, SBS112, SBS113, SBS115, | | |
| | | SBS116, SBS117, SBS118, SBS119, | | |
| | | SBS121, SBS123, SBS124, SBS126, | | |
| | | SBS128, SBS129 | | |

### Example 5—Discussion

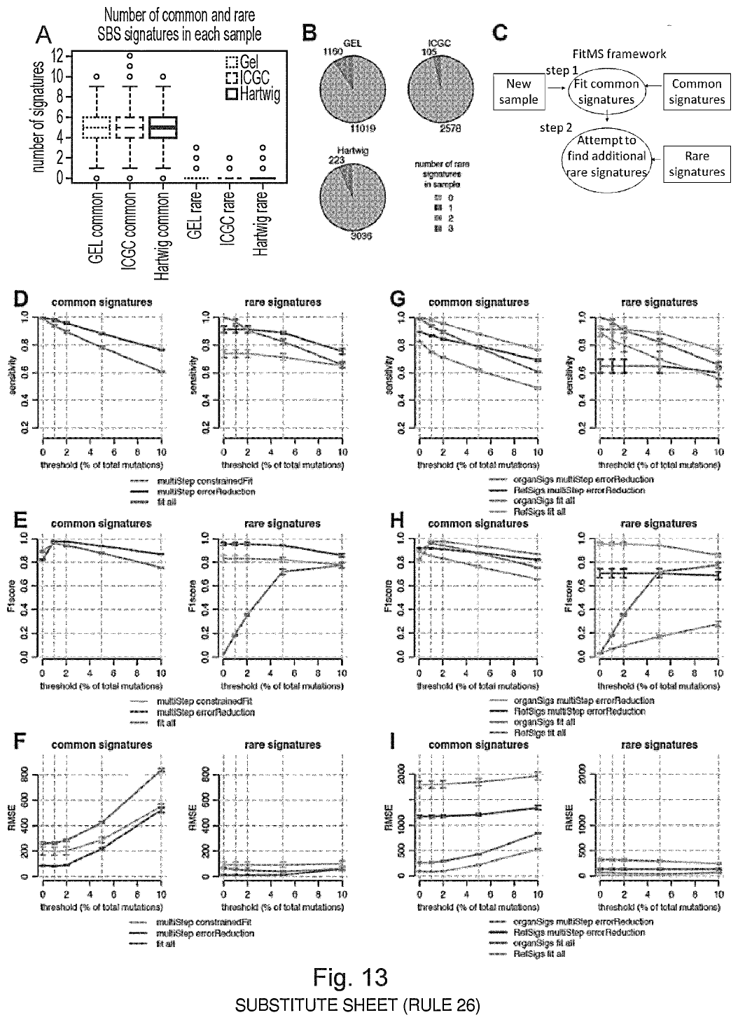

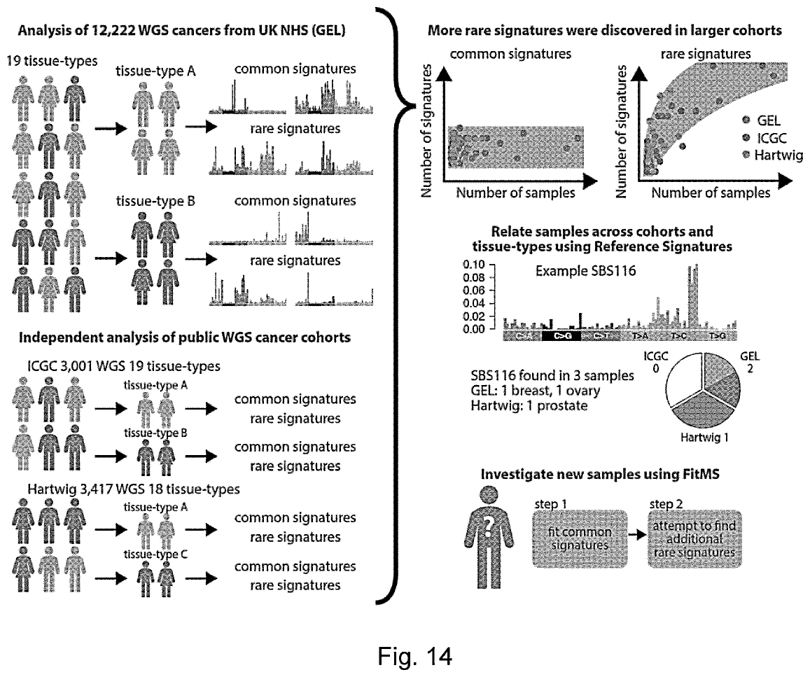

We report a comprehensive SBS and DBS signatures analysis of a large cohort of 18,640 WGS tumours (FIG. 14). Notably, majority of these samples were from patients recruited via the UK NHS (12,222) from across England, and the availability of open access WGS cancer data from ICGC and Hartwig Foundation were crucial for validation of findings. In all, 40 SBS and 18 DBS signatures that had not been previously reported, were revealed due to the increase in WGS cohort size. We were also able to confirm 42 previously reported SBS signatures and 9 previous DBS signatures. We introduce the notion of common and rare signatures for each tumor-type and observe that although the cohort of WGS cancers has increased substantially, most of the common signatures have been identified and many of the previously unreported signatures are low-frequency, rare processes. The landscape of signatures is thus likely to be saturating. The power to accurately discern mutational signatures is orders of magnitude greater using a pure WGS dataset when compared to other sequencing strategies. The genomic footprint for whole exomes (WES) is 100-fold lower and 2,000-4,000-fold lower in targeted sequencing (TS) experiments. Analyzing solely WGS cancers, rather than pooling data from diverse sequencing strategies, also avoids issues related to differing AT/GC representation in WES/TS data, which influence signature extractions.

Methodologically, several points are worthy to note. First, grouping samples by organs and focusing on common mutational profiles has produced signatures that are highly reproducible across cohorts. Removing atypical samples in the first extraction step is especially important for large cohorts, where very rare signatures may be present and could interfere with the accurate identification of common signatures. Second, the use of three large independent cohorts is crucial to validate signatures found in single organs, such as SBS120, and that could otherwise be mistaken for other signatures or considered artefactual. Third, while some signatures may have very similar 96-element SBS profiles to other well-known signatures, additional information, such as co-occurrence with DBS or signatures transcriptional/replication strand bias, can suggest a different etiology and help validate them as distinct signatures. Thus, deeper investigation can often show distinctions indicating diverse etiologies, a caveat that must be considered when using mutational signatures in future analyses.

From a biological perspective, it is essential to discriminate signatures that provide diagnostic insights or are therapeutically informative from other signatures, particularly when there are feature similarities between them. Notable examples deliberated earlier include distinguishing MBD4-compromised SBS96 from other signatures with CpG propensity or correctly differentiating signatures that occur predominantly at CCN and TCN from UV-related SBS7a. This is greatly improved by the methods described herein.

Additionally, we highlight endogenous signatures indicative of pathway defects that are detectable using WGS signatures but for which a genetic driver cannot be identified. It is worthy of note that a causal genetic event could not be detected for one in two cases with MMRd and one in two cases with HR-deficiency, indicating that signature analysis has increased sensitivity to identify these defects than examining mutations in selected genes, using targeted sequencing strategies. Furthermore, an agnostic WGS approach to tumour characterization will help reveal abnormalities that we currently neither seek nor detect using customary diagnostic pathways. For example, we found MMRd associated signatures in many tumour types with a frequency lower than 1% including stomach, prostate, pancreas, ovary, NET, lung, kidney, oropharyngeal, CNS, breast, sarcoma and bladder cancers. Given reported therapeutic relationships between MMRd phenotypes and immune checkpoint inhibitors (37-39), from a personalized pan-cancer therapeutics perspective, many of these patients could be eligible for treatment options that would otherwise not be available to them.

We note that many of the previously unreported signatures have no known etiology currently. This is not surprising because of the complexity of drawing causal relationships, particularly for endogenous signatures, which can be the outcome of multiple co-occurring events. For example, a gene defect in MBD4 could convert the ubiquitous C>T at CpG into a hypermutator phenotype (SBS96), or a pathophysiological state such as replication stress could amplify APOBEC-related SBS13. Some endogenous signatures may only manifest as part of an adaptive response to stressful stimuli. For example, SBS17, defined by T>G and T>C mutations, was reported in mouse cells that have been through immortalization, in normal human cells treated with 5-FU (40), and in a wide variety of cancers. Thus, many of the signatures of unknown etiology could be due to not just a single gene defect but multi-gene or complex pathway abnormalities (41) and/or may become overt following an adaptive response to cellular stress. Further work will be required to fully comprehend the causes of many cancer mutational signatures.

As our knowledge base increases, the complexity of assigning genetic causality to signatures is evident in examples such as the OGG1 polymorphic risk allele, where some patients exhibit SBS108 clearly, and others do not. Looking forward, alternative strategies may be needed to detect the contribution of moderate and lower penetrance germline risk alleles to somatic signatures in large cohorts.

Notably, the present analysis introduces the concept of common versus rare signatures within each tumor-type. It highlights how an increased number of samples may help discern common signatures that occur at low levels for specific tumor-types. Greater sample numbers may also help unveil signatures that occur at a low frequency in the population. Crucially, the availability of independent, open-access datasets such as from the ICGC and HMF has been instrumental in corroborating these common and rare signatures identified within the GEL dataset. While it is far simpler to discuss signatures as unifying reference patterns across all organs, it is important to note that these are mathematical reference patterns, an average of many extractions, and not necessarily an accurate biological representation of the process in any given tissue. For users seeking to learn what signatures may be present in a new set of samples, it may be more advisable to use organ-specific signatures to perform an analysis rather than mathematically-averaged signatures.

Thus, here we suggest a strategy of using mutational signatures, which considers the biological insights and complexities described in this work. FitMS invites the user to use common organ-specific signatures in the first instance, followed by hunting down the presence of rare signatures subsequently (FIG. 9, FIG. 14).

### REFERENCES

1. H. Sung et al., Global Cancer Statistics 2020: GLOBOCAN Estimates of Incidence and Mortality Worldwide for 36 Cancers in 185 Countries. CA Cancer J Clin 71, 209-249 (2021).

2. M. R. Stratton, P. J. Campbell, P. A. Futreal, The cancer genome. Nature 458, 719-724 (2009).

3. D. R. Bentley et al., Accurate whole human genome sequencing using reversible terminator chemistry. Nature 456, 53-59 (2008).

4. T. Helleday, S. Eshtad, S. Nik-Zainal, Mechanisms underlying mutational signatures in human cancers. Nat Rev Genet 15, 585-598 (2014).

5. L. B. Alexandrov et al., Signatures of mutational processes in human cancer. Nature 500, 415-421 (2013).

6. S. Nik-Zainal et al., Mutational processes molding the genomes of 21 breast cancers. Cell 149, 979-993 (2012).

7. C. Turnbull, Introducing whole-genome sequencing into routine cancer care: the Genomics England 100 000 Genomes Project. Ann Oncol 29, 784-787 (2018).

8. J. Ma, J. Setton, N. Y. Lee, N. Riaz, S. N. Powell, The therapeutic significance of mutational signatures from DNA repair deficiency in cancer. Nat Commun 9, 3292 (2018).

9. S. Nik-Zainal et al., Landscape of somatic mutations in 560 breast cancer whole-genome sequences. Nature 534, 47-54 (2016).

10. C. Ganini et al., Global mapping of cancers: The Cancer Genome Atlas and beyond. Mol Oncol, (2021).

11. I. T. P.-C. A. o. W. G. Consortium, Pan-cancer analysis of whole genomes. Nature 578, 82-93 (2020).

12. P. Priestley et al., Pan-cancer whole-genome analyses of metastatic solid tumours. Nature 575, 210-216 (2019).

13. E. Turro et al., Whole-genome sequencing of patients with rare diseases in a national health system. Nature 583, 96-102 (2020).

14. L. B. Alexandrov et al., The repertoire of mutational signatures in human cancer. Nature 578, 94-101 (2020).

15. A. R. J. Lawson et al., Extensive heterogeneity in somatic mutation and selection in the human bladder. Science 370, 75-82 (2020).

16. S. M. A. Islam et al., Uncovering novel mutational signatures by <em>de novo</em> extraction with SigProfilerExtractor. bioRxiv, 2020.2012.2013.422570 (2021).

17. A. Degasperi et al., A practical framework and online tool for mutational signature analyses show inter-tissue variation and driver dependencies. Nat Cancer 1, 249-263 (2020).

18. O. Pich et al., The mutational footprints of cancer therapies. Nat Genet 51, 1732-1740 (2019).

19. P. S. Robinson et al., Elevated somatic mutation burdens in normal human cells due to defective DNA polymerases. bioRxiv, 2020.2006.2023.167668 (2020).

20. C. Swanton, N. McGranahan, G. J. Starrett, R. S. Harris, APOBEC Enzymes: Mutagenic Fuel for Cancer Evolution and Heterogeneity. Cancer Discov 5, 704-712 (2015).

21. M. A. Sanders et al., MBD4 guards against methylation damage and germ line deficiency predisposes to clonal hematopoiesis and early-onset AML. Blood 132, 1526-1534 (2018).

22. B. Li et al., Therapy-induced mutations drive the genomic landscape of relapsed acute lymphoblastic leukemia. Blood 135, 41-55 (2020).

23. B. S. Strauss, The ‘A rule’ of mutagen specificity: a consequence of DNA polymerase bypass of non-instructional lesions? Bioessays 13, 79-84 (1991).

24. M. Rodrigues et al., Outlier response to anti-PD1 in uveal melanoma reveals germline MBD4 mutations in hypermutated tumours. Nat Commun 9, 1866 (2018).

25. X. Zou et al., A systematic CRISPR screen defines mutational mechanisms underpinning signatures caused by replication errors and endogenous DNA damage. Nat Cancer 2, 643-657 (2021).

26. C. Pilati et al., Mutational signature analysis identifies MUTYH deficiency in colorectal cancers and adrenocortical carcinomas. J Pathol 242, 10-15 (2017).

27. A. Viel et al., A Specific Mutational Signature Associated with DNA 8-Oxoguanine Persistence in MUTYH-defective Colorectal Cancer. EBioMedicine 20, 39-49 (2017).

28. P. Garre et al., Analysis of the oxidative damage repair genes NUDT1, OGG1, and MUTYH in patients from mismatch repair proficient HNPCC families (MSS-HNPCC). Clin Cancer Res 17, 1701-1712 (2011).

29. N. J. Haradhvala et al., Distinct mutational signatures characterize concurrent loss of polymerase proofreading and mismatch repair. Nat Commun 9, 1746 (2018).

30. H. Davies et al., HRDetect is a predictor of BRCA1 and BRCA2 deficiency based on mutational signatures. Nat Med 23, 517-525 (2017).

31. P. Polak et al., A mutational signature reveals alterations underlying deficient homologous recombination repair in breast cancer. Nat Genet 49, 1476-1486 (2017).

32. J. Staaf et al., Whole-genome sequencing of triple-negative breast cancers in a population-based clinical study. Nat Med 25, 1526-1533 (2019).

33. J. E. Kucab et al., A Compendium of Mutational Signatures of Environmental Agents. Cell 177, 821-836 e816 (2019).

34. E. Pleasance et al., Pan-cancer analysis of advanced patient tumours reveals interactions between therapy and genomic landscapes. Nature Cancer 1, 452-468 (2020).

35. C. Pleguezuelos-Manzano et al., Mutational signature in colorectal cancer caused by genotoxic pks(+) E. coli. Nature 580, 269-273 (2020).

36. P. J. Dziubanska-Kusibab et al., Colibactin DNA-damage signature indicates mutational impact in colorectal cancer. Nat Med 26, 1063-1069 (2020).

37. D. T. Le et al., Mismatch repair deficiency predicts response of solid tumours to PD-1 blockade. Science 357, 409-413 (2017).

38. A. Marabelle et al., Efficacy of Pembrolizumab in Patients With Noncolorectal High Microsatellite Instability/Mismatch Repair-Deficient Cancer: Results From the Phase II KEYNOTE-158 Study. J Clin Oncol 38, 1-10 (2020).

39. H. Veeraraghavan et al., Machine learning-based prediction of microsatellite instability and high tumour mutation burden from contrast-enhanced computed tomography in endometrial cancers. Sci Rep 10, 17769 (2020).

40. S. Christensen et al., 5-Fluorouracil treatment induces characteristic T>G mutations in human cancer. Nat Commun 10, 4571 (2019).

41. G. Rospo et al., Evolving neoantigen profiles in colorectal cancers with DNA repair defects. Genome Med 11, 42 (2019).

42. S. Morganella et al., The topography of mutational processes in breast cancer genomes. Nat Commun 7, 11383 (2016).

43. See supplementary materials.

44. A. Degasperi et al., Mutational signatures in whole-genome-sequenced cancers of the UK national health service, Mutational Signatures Data. Zenodo, doi: 10.5281/zenodo.5571551 (2021).

45. A. Degasperi et al., Mutational signatures in whole-genome-sequenced cancers of the UK national health service, Supplementary Code S1 and S2. Zenodo, doi: 10.5281/zenodo.5570307 (2021).

46. D. D. Lee, H. S. Seung, in Advances in Neural Information Processing Systems 13—Proceedings of the 2000 Conference, NIPS 2000. (Neural information processing systems foundation, 2001).

47. B. B. Campbell et al., Comprehensive Analysis of Hypermutation in Human Cancer. Cell 171, 1042-1056 e1010 (2017).

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Various modifications and variations of the described compositions, methods, and uses of the technology will be apparent to those skilled in the art without departing from the scope and spirit of the technology as described. Any sub-titles herein are included for convenience only, and are not to be construed as limiting the disclosure in any way.

Unless context dictates otherwise, the descriptions and definitions of the features set out above are not limited to any particular aspect or embodiment of the invention and apply equally to all aspects and embodiments which are described.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

Other aspects and embodiments of the invention provide the aspects and embodiments described above with the term “comprising” replaced by the term “consisting of” or “consisting essentially of”, unless the context dictates otherwise.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

LENGTHY TABLES
The patent application contains a lengthy table section. A copy of the table is available in electronic form from the USPTO web site (https://seqdata.uspto.gov/?pageRequest=docDetail&DocID=US20250191681A1). An electronic copy of the table will also be available from the USPTO upon request and payment of the fee set forth in 37 CFR 1.19(b)(3).

1. A method of characterising a DNA sample, the method including the steps of:
obtaining a mutational catalogue for the sample, wherein a mutational catalogue comprises counts of mutations in a plurality of predetermined categories;
obtaining a mutational signatures catalogue comprising a first set of one or more mutational signatures and a second set of one or more mutational signatures;
determining a first set of exposures of the sample to the mutational signatures in the first set of mutational signatures;
identifying at least one of the mutational signatures in the second set of mutational signatures that is likely to be present in the sample using the results of the determining; and
providing an indication of which of the mutational signatures in the mutational signatures catalogue is present in the sample.

2. The method of claim 1, wherein the mutational signatures in the first set are more likely to be present in the sample than the mutational signatures in the second set, and/or wherein identifying at least one of the mutational signatures in the second set of mutational signatures that is likely to be present in the sample using the results of the determining comprises identifying one or more candidate mutational signatures in the second set that improve the fit of a mutational catalogue comprising the first set of mutational signature and the one or more candidate mutational signatures, compared to a mutational signature catalogue that does not comprise the candidate mutational signature.

3. The method of claim 1 or claim 2, wherein identifying at least one of the mutational signatures in the second set of mutational signatures that is likely to be present in the sample using the results of the determining comprises:
determining a second set of exposures of the sample to the mutational signatures in the first set of mutational signatures and a candidate mutational signature in the second set of mutational signatures; and
determining that the candidate mutational signature in the second set of mutational signatures is likely to be present in the sample if the reconstruction error between the mutational catalogue and a reconstructed mutational catalogue associated with the second set of exposures satisfies one or more predetermined criteria, optionally wherein the one or more predetermined criteria are selected from: the difference between the reconstruction error associated with the first set of exposures and the reconstruction error associated with the second set of exposures being below a predetermined value, and the candidate mutational signature being associated with the highest difference between the reconstruction error associated with the first set of exposures and the reconstruction error associated with the second set of exposures amongst all mutational signatures in the second set of mutational signatures.

4. The method of claim 1 or claim 2, wherein identifying at least one of the mutational signatures in the second set of mutational signatures that is likely to be present in the sample using the results of the determining comprises:
Determining the residual between the mutational catalogue for the sample and a reconstructed catalogue corresponding to the determined exposures; and
Determining that a candidate mutational signature in the second set of mutational signatures is likely to be present in the sample if the similarity between the residual and the candidate mutational signature satisfies one or more predetermined criteria; optionally wherein the one or more predetermined criteria are selected from: the similarity being above a predetermined threshold, and the similarity being highest amongst all mutational signatures in the second set of mutational signatures.

5. The method of any preceding claim, further comprising determining a further set of exposures of the sample to the mutational signatures in the first set of mutational signatures and any mutational signatures in the second set of mutational signatures that is identified as likely to be present in the sample using the results of the determining.

6. The method of claim 5, wherein the method further comprises excluding exposures that represent a proportion of the total sample mutations below a predetermined threshold, optionally wherein the predetermined threshold is between 0 and 10%, about 1%, about 2%, about 3%, about 5% or about 10%.

7. The method of claim 5 or claim 6, further comprising identifying one or more mutational processes present in the sample using at least one of the further set of exposures, optionally wherein identifying one or more mutational processes present in the sample comprises determining whether the exposures are indicative of the presence of a signature associated with the one or more mutational processes or a signature that maps to a signature associated with the one or more mutational processes.

8. The method of any preceding claim, wherein identifying at least one of the mutational signatures in the second set of mutational signatures that is likely to be present in the sample using the results of the determining comprises identifying a single candidate mutational signature in the second set that improves the fit of a mutational catalogue comprising the first set of mutational signature and the candidate mutational signature, compared to a mutational signature catalogue that does not comprise the candidate mutational signature.

9. The method of any preceding claim, wherein the signatures in the first and/or second set of mutational signatures are mutational signatures that have been extracted from organ-specific cohorts of samples.

10. The method of any preceding claim, wherein the first set of mutational signatures are mutational signatures that are specific to the organ from which the sample originates.

11. The method of any preceding claim, wherein the second set of mutational signatures are mutational signatures that are not already represented in the first set of mutational signatures and that have been extracted in at least two independent extractions from respective cohorts of samples, optionally wherein the two independent extractions are extractions performed on two different organ-specific cohorts.

12. The method of any preceding claim, wherein the sample is a tumor sample or a sample derived therefrom, optionally wherein the sample is from a tumour type selected from: skin, lung, stomach, colorectal, bladder, liver, uterus, ovary, biliary, kidney, pancreas, breast, prostate, bone/soft-tissue, central nervous system (CNS), lymphoid, oropharyngeal, neuroendocrine tumors (NET), and myeloid tumour and/or wherein an organ-specific cohort is a cohort comprising samples selected from a single one of: skin, lung, stomach, colorectal, bladder, liver, uterus, ovary, biliary, kidney, pancreas, breast, prostate, bone/soft-tissue, central nervous system (CNS), lymphoid, oropharyngeal, neuroendocrine tumors (NET), and myeloid tumour.

13. The method of any preceding claim, wherein the mutational catalogue for the sample has been derived from sequence data for the sample or wherein the method comprises obtaining sequence data for the sample and deriving the mutational catalogue by counting mutations within each of the predetermined categories, or wherein the mutational catalogue and/or the mutational signatures catalogue has been determined from whole genome sequencing data, and/or wherein the mutational catalogue comprises the counts of the number of somatic mutations for each of a plurality of categories of single base substitutions or double base substitutions, optionally wherein the mutational catalogue is a 96 channel SBS profile or a 78 channel DBS profile, or wherein the mutational catalogue comprises the counts of the number of somatic mutations for each of a plurality of categories of insertions and/or deletions, or wherein the mutational catalogue comprises the counts of the number of somatic mutations for each of a plurality of categories of rearrangements.

14. The method of any preceding claim, wherein the mutational signatures in the first set and/or in the second set are mutational signatures that have been validated by cross reference with at least one independently extracted mutational signature catalogue.

15. The method of any preceding claim, wherein the mutational signatures in the first set of signatures have been identified by extracting mutational signatures from a cohort of samples which has been separated into a first group and a second group of samples, the first group comprising samples with mutational profiles that are more common than the mutational profiles of the samples in the second group, wherein the first set of signatures have been identified by extracting mutational signatures from the cohort of samples excluding the second group of samples.

16. The method of claim 15, wherein the mutational signatures in the second set of signatures have been identified in one or more cohorts of samples that are different from the cohort of samples from which the first set of signatures have been identified, or by extracting mutational signatures from one or more samples in the second group of samples using the first set of mutational signatures as constant in the extraction process.

17. The method of claim 15 or claim 16, wherein the first set of signatures have been identified by extracting mutational signatures from a cohort of samples which has been separated into a first group and a second group of samples by clustering the mutational catalogues for the samples, optionally using hierarchical clustering and/or 1-cosine distance as a similarity measure.

18. The method of any of claims 15 to 17, wherein one or more signatures in the second set of signatures were identified using a process comprising:
separating a cohort of samples into a first group and a second group of samples, the first group comprising samples with mutational catalogue that are more common than the mutational profiles of the samples in the second group,
identifying a first set of mutational signatures by extracting mutational signatures from the cohort of samples excluding the second group of samples,
identifying one or more samples in the second group of samples with mutational profiles based on the reconstruction error associated with a mutation catalogue reconstructed using the first set of mutational signatures, optionally wherein the reconstruction error for a sample is the sum of absolute deviations between the mutational catalogue c of the sample and the reconstructed mutational catalogue Se obtained by fitting a mutational signature catalogue to the mutational profile of the sample, divided by the total mutations in the mutational catalogue of the sample, and
extracting one or more signatures from the identified one or more samples in the second group of samples.

19. The method of claim 18, wherein identifying one or more samples in the second group of samples with mutational profiles based on the reconstruction error associated with a mutation catalogue reconstructed using the first set of mutational signatures comprises:
for each sample in the second group of samples, obtaining a sample residual error by estimating the sample exposures obtained by fitting the first set of mutational signatures to the mutational profile for the sample and using a least square estimation with a constraint that the difference between the observed and reconstructed catalogues should be above a predetermined threshold applied to the sum of coefficients in the reconstructed catalogue; and
clustering the sample residual errors for the samples in the second group of samples, wherein the samples in a particular cluster are identified and used for signature extraction.

20. The method of claim 19, wherein one or more signatures in the second set of signatures were identified using a process further comprising for each cluster of samples, extracting one signature using an extraction process constrained to use the signatures in the first set of signatures as constant, optionally wherein the extraction process using NMF, where the signature matrix S contains the first set of signatures as constants, and one additional column that is estimated to contain the new signature.

21. The method of any of claims 15 to 21, wherein the one or more signatures in the second set of signatures have been identified by:
performing a process as described in any of claims 15 to 21 independently on at least two different cohorts of samples; and
selecting signatures that are identified in at least two of the different cohorts.

22. The method of any of claims 15 to 21, wherein extracting a first set of signatures comprises extracting between 5 and 10 SBS in a cohort of samples, preferably an organ-specific cohort of samples, and/or wherein extracting a second set of signatures comprises extracting between 0 and 21 SBS in said cohort of samples, and/or wherein extracting a first set of signatures comprises extracting between 1 and 5 DBS in a cohort of samples, preferably an organ-specific cohort of samples, and/or wherein extracting a second set of signatures comprises extracting between 0 and 15 DBS in said cohort of samples.

23. The method of any preceding claim, wherein providing an indication of which of the mutational signatures in the mutational signatures catalogue is present in the sample comprises:
determining exposure to signatures in a mutational signature catalogue comprising the first set of signatures and at least one signature in the second set of signatures, and
mapping the exposures to a first set and/or a second set of mutational signatures to a reference set of mutational signatures and optionally identifying one or more mutational processes likely to be active in the sample based on the mapping, and/or using one or more exposures as an input to a method for determining whether the DNA sample is from a tumour that has a deficiency in a DNA repair pathway and/or using one or more exposures as an input to a method for determining whether the DNA sample is from a tumour that has a characteristic that is indicative or prognosis or response to therapy and/or providing the one or more exposures or metrics derived therefrom as part of a report characterising a tumour from which the DNA sample has been obtained.

24. A method of providing a mutational signature catalogue, the method comprising:
separating a cohort of samples into a first group and a second group of samples, the first group comprising samples with mutational profiles that are more common than the mutational profiles of the samples in the second group, and
extracting a first set of mutational signatures from the cohort of samples excluding the second group of samples.

25. A system comprising:
a processor; and
a computer readable medium comprising instructions that, when executed by the processor, cause the processor to perform the steps of the method of any of claims 1 to 24.
